SEQUENCE LISTING (27 sequences)

SEQ ID NO: 1 - DNA, 21 bp, unidentified
  source 1..21: mol_type = unassigned DNA
ccctccagcctctcctcgcat

SEQ ID NO: 2 - DNA, 32 bp, unidentified
  source 1..32: mol_type = unassigned DNA
caccccctccagcctctcctcgcatgcagcgc

SEQ ID NO: 3 - DNA, 32 bp, unidentified
  source 1..32: mol_type = unassigned DNA
caccccctccagcctctcctcgcatgcggcgc

SEQ ID NO: 4 - DNA, 21 bp, unidentified
  source 1..21: mol_type = unassigned DNA
catgtgtgggcgccgcatgcg

SEQ ID NO: 5 - DNA, 32 bp, unidentified
  source 1..32: mol_type = unassigned DNA
ctgccatgtgtgggcgctgcatgcgaggagag

SEQ ID NO: 6 - DNA, 32 bp, unidentified
  source 1..32: mol_type = unassigned DNA
ctgccatgtgtgggcgccgcatgcgaggagag

SEQ ID NO: 7 - RNA, 104 nt, unidentified
  source 1..104: mol_type = unassigned RNA
gnnnnnnnnnnnnnnnnnnnnngttttagtactctggaaacagaatctactaaaacaaggcaaaatgccgtgtttatctcgtcaacttgttggcgagatttttt

SEQ ID NO: 8 - RNA, 112 nt, unidentified
  source 1..112: mol_type = unassigned RNA
gnnnnnnnnnnnnnnnnnnnnngttttagtactctgtaatgaaaattacagaatctactaaaacaaggcaaaatgccgtgtttatctcgtcaacttgttggcgagatttttt

SEQ ID NO: 9 - RNA, 104 nt, unidentified
  source 1..104: mol_type = unassigned RNA
gnnnnnnnnnnnnnnnnnnnnngtttaagtactctggaaacagaatctacttaaacaaggcaaaatgccgtgtttatctcgtcaacttgttggcgagatttttt

SEQ ID NO: 10 - RNA, 151 nt, unidentified
  source 1..151: mol_type = unassigned RNA
gnnnnnnnnnnnnnnnnnnngtttttgtactctcaagatttaagtaactgtacaacgaaacttacacagttacttaaatcttgcagaagctacaaagataaggcttcatgccgaaatcaacaccctgtcattttatggcagggtgtttttt

SEQ ID NO: 11 - RNA, 151 nt, unidentified
  source 1..151: mol_type = unassigned RNA
gnnnnnnnnnnnnnnnnnnngtttgtgtactctcaagatttaagtaactgtacaacgaaacttacacagttacttaaatcttgcagaagctacaaagataaggcttcatgccgaaatcaacaccctgtcattttatggcagggtgtttttt

SEQ ID NO: 12 - RNA, 109 nt, unidentified
  source 1..109: mol_type = unassigned RNA
gnnnnnnnnnnnnnnnnnnngtttttgtactctcagaaatgcagaagctacaaagataaggcttcatgccgaaatcaacaccctgtcattttatggcagggtgtttttt

SEQ ID NO: 13 - RNA, 104 nt, unidentified
  source 1..104: mol_type = unassigned RNA
gnnnnnnnnnnnnnnnnnnngtttttgtactctgaaaagaagctacaaagataaggcttcatgccgaaatcaacaccctgtcattttatggcagggtgtttttt

SEQ ID NO: 14 - RNA, 106 nt, unidentified
  source 1..106: mol_type = unassigned RNA
gnnnnnnnnnnnnnnnnnnngtctttgtactctggtaccagaagctacaaagataaggcttcatgccgaaatcaacaccctgtcattttatggcagggtgtttttt

SEQ ID NO: 15 - RNA, 151 nt, unidentified
  source 1..151: mol_type = unassigned RNA
gnnnnnnnnnnnnnnnnnnnnnnngttgtagctccctttctcatttcggaaacgaaatgagaaccgttgctacaataaggccgtctgaaaagatgtgccgcaacgctctgccccttaaagcttctgctttaaggggcatcgtttatttttt

SEQ ID NO: 16 - RNA, 131 nt, unidentified
  source 1..131: mol_type = unassigned RNA
gnnnnnnnnnnnnnnnnnnnnnnngttgtagctccctttctcgaaagagaaccgttgctacaataaggccgtctgaaaagatgtgccgcaacgctctgccccttaaagcttctgctttaacgggctttttt

SEQ ID NO: 17 - RNA, 127 nt, unidentified
  source 1..127: mol_type = unassigned RNA
gnnnnnnnnnnnnnnnnnnnnnnngttgtagctccctttcgaaagaaccgttgctacaataaggccgtctgaaaagatgtgccgcaacgctctgccccttaaagcttctgctttaacgggctttttt

SEQ ID NO: 18 - RNA, 102 nt, unidentified
  source 1..102: mol_type = unassigned RNA
gnnnnnnnnnnnnnnnnnnnnnngttttagtccctgaaaagggactaaaataaagagtttgcgggactctgcggggttacaatcccctaaaaccgctttttt

SEQ ID NO: 19 - RNA, 100 nt, unidentified
  source 1..100: mol_type = unassigned RNA
gnnnnnnnnnnnnnnnnnnnnnngttttagtccctgaagggactaaaataaagagtttgcgggactctgcggggttacaatcccctaaaaccgctttttt

SEQ ID NO: 20 - DNA, 21 bp, unidentified
  source 1..21: mol_type = unassigned DNA
caagggcccatgagcggcaca

SEQ ID NO: 21 - DNA, 34 bp, unidentified
  source 1..34: mol_type = unassigned DNA
cgcccaagggcccatgagcggcacaaagaaacta

SEQ ID NO: 22 - DNA, 12 bp, unidentified
  source 1..12: mol_type = unassigned DNA
ggtttagagacg

SEQ ID NO: 23 - DNA, 12 bp, unidentified
  source 1..12: mol_type = unassigned DNA
cgtctcttttga

SEQ ID NO: 24 - DNA, 11 bp, unidentified
  source 1..11: mol_type = unassigned DNA
gtttagagacg

SEQ ID NO: 25 - DNA, 11 bp, unidentified
  source 1..11: mol_type = unassigned DNA
caaaagagacg

SEQ ID NO: 26 - RNA, 108 nt, unidentified
  source 1..108: mol_type = unassigned RNA
gnnnnnnnnnnnnnnnnnnnnngtttaagtactctgtagaaatacagaatctacttaaacaaggcaaaatgccgtgtttatctcgtcaacttgttggcgagatttttt

SEQ ID NO: 27 - RNA, 113 nt, unidentified
  source 1..113: mol_type = unassigned RNA
gnnnnnnnnnnnnnnnnnnnnngtttaagtactctggaaacagaatctacttaaacaaggcaggacttcggtcctgccgtgtttatctcgtcaacttgttggcgagatttttt

(19) United States
(12) Patent Application Publication
(10) Pub. No.: US 2024/0055077 A1
(43) Pub. Date: Feb. 15, 2024

(54) SYSTEM AND METHOD FOR PREDICTING ACTIVITY AND SPECIFICITY OF 17 SMALL Cas9s USING DEEP LEARNING

(71) Applicant: INDUSTRY-ACADEMIC COOPERATION FOUNDATION, YONSEI UNIVERSITY, Seoul (KR)

(72) Inventors: Hyongbum Henry KIM, Seoul (KR); Sangyeon SEO, Seoul (KR); Sungtae LEE, Daejeon (KR)

(73) Assignee: INDUSTRY-ACADEMIC COOPERATION FOUNDATION, YONSEI UNIVERSITY, Seoul (KR)

(21) Appl. No.: 18/319,071

(22) Filed: May 17, 2023

(30) Foreign Application Priority Data
May 17, 2022 (KR) 10-2022-0060290
May 16, 2023 (KR) 10-2023-0063272

(51) Int. Cl. G16B 40/00; G06N 3/0464

(52) U.S. Cl. CPC G16B 40/00; G06N 3/0464

(57) ABSTRACT

A system for predicting an activity of small Cas9 using deep learning, including a sequence input unit receiving input data on a guide sequence and target sequence of small Cas9, a predictive model generator generating a small Cas9 activity predictive model by performing deep learning for learning a relationship between small Cas9 activity data obtained from the input data on the guide sequence and target sequence of small Cas9 received from the sequence input unit and features that affect small Cas9 activity, a candidate target sequence input unit receiving candidate target sequence of small Cas9, and an activity predictor predicting small Cas9 activity by applying candidate target sequence input in the candidate target sequence input unit to the predictive model generated in the predictive model generator.

### CROSS-REFERENCE TO RELATED APPLICATION

This application is based on and claims priority under 35 U.S.C. § 119 to Korean Patent Application Nos. 10-2022-0060290, filed on May 17, 2022, and 10-2023-0063272, filed on May 16, 2023 in the Korean Intellectual Property Office, the disclosure of which is incorporated by reference herein in its entirety,

### REFERENCE TO SEQUENCE LISTING

The instant application contains a Sequence Listing which has been submitted electronically in XML format and is hereby incorporated by reference in its entirety. Said XML copy, created on Aug. 21, 2023, is named 548265US_SL.xml and is 26,321 bytes in size.

### BACKGROUND

### 1. Field

The present invention relates to a system for predicting the activity of small Cas9 using deep learning.

### 2. Description of the Related Art

Small-sized Cas9s are advantageous for delivery, especially for in vivo applications, and various small Cas9 orthologues and variants (for brevity, small Cas9s) have been reported. However, selecting the optimal small Cas9 for use at a specific target sequence can be confusing. Here we systematically compared the activities of 17 small Cas9s at thousands of target sequences. For each small Cas9, we characterized the protospacer adjacent motif and determined optimal single guide RNA expression formats and scaffold sequence. High-throughput comparative analyses showed a high-activity group containing sRGN3.1, SlugCas9, SaCas9, SauriCas9, Sa-SlugCas9, SaCas9-KKH, eSaCas9, and efSaCas9, and a low-activity group containing SauriCas9-KKH, SlugCas9-HF, SaCas9-HF, SaCas9-KKH-HF, St1Cas9, Nm1Cas9, enCjCas9, CjCas9, and Nm2Cas9. We also developed DeepSmallCas9, a set of computational models predicting the activities of small Cas9s at matched and mismatched target sequences. These computational models, together with this new understanding about the small Cas9s, provide a useful guide to their use.

### SUMMARY

Provided is a system for predicting the activity of small Cas9 using deep learning.

Provided is a method for predicting the activity of small Cas9 using deep learning.

Provided is a computer-readable recording medium having recorded thereon a program for causing a computer to execute a method for predicting the activity of small Cas9 using deep learning.

Provided is a method for providing information on small Cas9 and sgRNA that can specifically remove human single nucleotide mutations using deep learning.

Additional aspects will be set forth in part in the description which follows and, in part, will be apparent from the description, or may be learned by practice of the presented embodiments of the disclosure.

Provided is a system for predicting the activity of small Cas9 using deep learning.

In detail, one embodiment of the present invention provides a system for predicting the activity of small Cas9 using deep learning, comprising: a sequence input unit receiving input data on the guide sequence and target sequence of small Cas9; a predictive model generator generating a small Cas9 activity predictive model by performing deep learning for learning the relationship between small Cas9 activity data obtained from the input data on the guide sequence and target sequence of small Cas9 received from the sequence input unit and features that affect small Cas9 activity; a candidate target sequence input unit receiving candidate target sequence of small Cas9; and an activity predictor predicting small Cas9 activity by applying candidate target sequence input in the candidate sequence input unit to a predictive model generated in the predictive model generator.

We extensively characterized PAM compatibilities and determined optimal sgRNA expression formats and scaffold sequences, and measured editing activities at thousands of matched and mismatched target sequences for 17 small Cas9s. Interestingly, we found that both the general activities and specificities of sRGN3.1 and SlugCas9 were higher than those of SpCas9, the most widely used Cas9. Given that the PAM compatibilities of these two small Cas9s are very similar to that of SpCas9, these two Cas9s could frequently be recommend over SpCas9 as programmable nucleases considering their higher activities, higher specificities, and smaller sizes. DeepSmallCas9 is designed to predict the activities of these 17 Cas9s at specified target sequences, By using DeepSmallCas9, researchers can choose the appropriate small Cas9 and sgRNA for their genome editing projects. In addition, unlike previously developed computational models that predict the activities of genome editing tools, DeepSmallCas9 can predict the activities of the small Cas9s at mismatched as well as matched target sequences. Thus, users can select the small Cas9 and sgRNA predicted to have the highest activity at the desired target sequences and the lowest activities at potential off-target sites.

We used lentiviral vectors to express small Cas9s and sgRNAs in HEK293T cells. As shown by our lab and others, CRISPR nuclease activity-predicting computational models, which were developed based on information from experiments involving lentiviral expression of Cas9 (or Cas12a) and sgRNA in HEK293T cells, are also useful for predicting the results of editing performed under different conditions. Such variable conditions include transient transfection of Cas9- and sgRNA-encoding plasmids in cell types other than HEK293T.

We also observed that the relative activities of small Cas9 and sgRNA pairs were similar across different cell lines. Thus, we expect that our findings from this study should be applicable to genome editing performed using untested conditions, although slightly or significantly different results are possible under untested experimental settings, especially when RNA or ribonuclear protein complexes are used to deliver the small Cas9s and sgRNAs. Such delivery methods were not tested even in the previous high-throughput studies related to the current study,

The transfection of Cas9-encoding plasmids into cultured cells and the transduction of Cas9-encoding AAV vectors in animal models are the most frequently used delivery methods for biological research and therapeutic applications. Comparisons in which the same number of DNA molecules encoding the Cas9 protein and sgRNA are delivered for all tested Cas9s allow the optimal small Cas9 and sgRNA pair for these approaches to be determined, so that the Cas9 activities at matched and mismatched targets are maximal and minimal, respectively. Thus, in this study, we delivered the same number (one copy per cell) of DNAs encoding the small Cas9 and sgRNA across all small Cas9s. However, the expressed Cas9 protein levels varied, which could be attributable to possible differences in protein stability and/or differences in codon usage that can affect protein expression. Although we used codons suggested by GenScript, the differences between smallCas9 amino acid sequences inevitably result in different codons. The elucidation of the exact mechanisms underlying these differential protein levels would require additional studies.

We did not evaluate other small Cas9s such as SpaCas9* (derived from Streptococcus pasteurianus), GeoCas9, CdCas9, Nm3Cas9, DfCas9, PpCas9, SpaCas9** (derived from Staphylococcus pasteuri), SmiCas9, or ShyCas9 due to their previously reported relatively lower efficiencies and/or extremely low PAM compatibilities of these small Cas9s. In addition, this study did not involve small Cas12s, some of which (e.g., AsCas12f1 and Un1Cas12f1) are much smaller than small Cas9s (FIG. 6). The results from our extensive characterization of 17 small Cas9s, together with DeepSmallCas9, should be useful for a broad range of genome editing studies involving these small Cas9s,

As used herein, the term “Cas9” or “Cas9 protein” refers to a major protein element of the CRISPR/Cas9 system, and the Cas9 protein forms a complex with CRISPR RNA (crRNA) and trans-activating crRNA (tracrRNA) to form activated endonuclease or nickase. Information about the Cas9 protein or genes thereof may be obtained from a known database such as GenBank of National Center for Biotechnology Information (NCBI), but any Cas9 protein having target-specific nuclease activity together with guide sequence may be included in the scope of the disclosure. In addition, the Cas9 protein may be bound with a protein transduction domain. The protein transduction domain may be poly-arginine or HIV TAT protein, but is not limited thereto. Furthermore, an additional domain may be suitably bound to the Cas9 protein by those skills in the art according to the intended use.

As used herein, the term “small Cas9” refers to Cas9 and variants thereof having a appropriately small size for delivering both CRISPR nuclease and its sgRNA using a single AAV vector. Small Cas9s can facilitate mRNA production and lipid nanoparticle (LNP)-mediated delivery, another promising delivery method for genome editing tools. Delivery of a small Cas9 using a single AAV vector or LNPs would be especially useful in cases in which disruptions of target sequences can ameliorate diseases or medical conditions

The small Cas9 may be any one selected from the group consisting of sRGN3.1, SlugCas9, SaCas9, SauriCas9, Sa-SluaCas9, SaCas9-KKH, eSaCas9, efSaCas9, SauriCas9-KKH, SlugCas9-HF, SaCas9-HF, SaCas9- KKH-HF, St1Cas9, Nm1Cas9, enCjCas9, CjCas9 ; and Nm2Cas9. The “SaCas9” may refer to SaCas9 expressed with a sequence used for expression of SaCas9 in the initial study of SaCas9-KKH, and “SaCas9*” may refer to SaCas9 expressed using a codon-optimized sequence recommended by GenScript.

As used herein, the term “guide sequence” or “guide RNA” refers to an RNA that is specific to a target sequence, and may be composed of a crRNA complementary to the target sequence and a tracrRNA for Cas9-binding. It complementarily binds to Cas9 and the target sequence in whole or in part to form a complex and serves to guide Cas9 to the target sequence,

In general, the guide RNA refers to a dual RNA composed of CRISPR RNA (crRNA) and trans-activating crRNA (tracrRNA) or a single-guide RNA (sgRNA), or refers to a form that includes a first region including a sequence complementary to all or part of a sequence in a target DNA, and a second region including a sequence interacting with an RNA-guided nuclease, but any form where an RNA-guided nuclease may have activity in a target sequence may be included in the scope of the disclosure without limitation. In addition, the guide RNA may include a scaffold sequence which helps the attachment of an RNA-guided nuclease.

As used herein, the term “target sequence” refers to a nucleotide sequence expected to be targeted by a small Cas9. In detail, the target sequence is a sequence that a small Cas9 is expected to target through a guide RNA, and may be a known sequence on which the small Cas9 exhibits an activity, or may be a sequence arbitrarily designed based on a sequence that one of skill in the art using the system of the disclosure to analyze, but any sequence that is to be analyzed as the small Cas9 exhibits or is expected to exhibit an activity thereon may be included in the scope of the disclosure without limitation.

The target sequence may include a protospacer adjacent motif (PAM) sequence and a protospacer sequence. In detail, the target sequence may include matched targets and targets with mismatches, insertions, or deletions with all types of PAMs (primary, secondary, or inactive PAMs)

As used herein, the term “sequence input unit” refers to a component that is included in a system for predicting the activity of a small Cas9 using deep learning, and is configured to receive an input of the target sequence.

As used herein, the term “data on the guide sequence and target sequence of small Cas9” may be existing known activity data, or may be activity data directly obtained by any method that may be appropriately adopted by one of skill in the art, and for the purpose of the disclosure, any method of obtaining data may be used as long as data for generating an activity prediction model capable of predicting the activity of a small Cas9 is obtained.

The term “small Cas9 activity data” corresponds to data for extracting and learning the relationship between a particular target sequence and the small Cas9 activity, and the system of the disclosure may generate a model for predicting the activity of small Cas9 by using the activity data.

The features that affect the small Cas9 activity may include information on the melting temperature (Tm) calculated in different regions of the target sequence, the number of G or C nucleotides in the spacer and protospacer, the minimum free energy (MFE) of the spacer and sgRNA, location and type of mismatch between the guide sequence and the protospacer sequence.

In addition, the features that affect the small Cas9 activity may further include information on the indel frequency of the target sequence.

The indel frequency is calculated through Equation 1 below:

$$Indel frequency ( \% ) = \frac{\begin{matrix}Indel read counts - \\ ( Total read \times Background indel frequency )\end{matrix}}{\begin{matrix}Total read counts - \\ ( Total read \times Background indel frequency )\end{matrix}} \times 100 \qquad [ Equation 1 ]$$

As used herein, the term “deep learning” refers to artificial intelligence (AI) technology that allows computers to think and learn like humans, and allows machines to learn and solve complex nonlinear problems on their own based on the artificial neural network theory. By using deep learning technology, it is possible to enable computers to recognize, infer, and judge on their own even when humans do not set all criteria for judgement, and thus to be widely used for voice and image recognition, image analysis, and the like. In other words, deep learning may be defined as a set of machine learning algorithms that attempt high-level abstractions (summarizing key content or functions in large amounts of data or complex materials) through a combination of several nonlinear transformation methods.

The predictive model generator may generate a model for predicting the activity of small Cas9 through a step of performing deep learning based on a convolutional neural network (CNN).

In detail, the step of performing deep learning based on the convolutional neural network may include connecting the small Cas9 activity data and the features that affect the small Cas9 activity.

The small Cas9 activity data may be obtained by a method including: infecting a cell line expressing small Cas9 with a lentiviral vector or library containing oligonucleotides, each comprising a guide sequence and its corresponding target sequence; performing deep sequencing by using DNA obtained from the cells into which the small Cas9 and lentiviral vector or library have been introduced; and measuring the indel frequency data from the data obtained by deep sequencing.

The term “predictive model generator” refers to a component capable of learning the relationship between the features that affect the small Cas9 activity and the small Cas9 activity by using the small Cas9 activity data input through the sequence input unit. The predictive model generator generates predictive models based on the learned information. Accordingly, a user may predict the small Cas9 activity by using the predictive models.

As used herein, the term “candidate target sequence of small Cas9” refers to a target nucleotide sequence whose small Cas9 activity is to be analyzed or predicted. The candidate target sequence may be derived from the genome sequence of a subject in which small Cas9 activity is to be confirmed, or may be any sequence designed and synthesized by a method known in the art, but its type is not limited within the range that the sequence may be applied to the system of the present disclosure to predict small Cas9 activity.

The candidate target sequence may include a protospacer adjacent motif (PAM) sequence and a protospacer sequence.

The “candidate target sequence input unit” is a component of the system for predicting the activity of small Cas9 for receiving an input of a candidate target sequence.

The “activity predictor” is a component that predicts small Cas9 activity, by applying the candidate target sequence input through the candidate sequence input unit to a model for predicting the activity of small Cas9 built by a preset method.

The system for predicting the activity of small Cas9 may further include an output unit for outputting small Cas9 activity score predicted by the activity predictor. In detail, the information on small Cas9 activity output by the output unit may be represented by a calculated value of the small Cas9 activity or a relative value to a preset reference value, but a form or type of the output information is not limited. For example, the information on small Cas9 activity may be output visually or audibly.

Proviede is a method for predicting the activity of small Cas9 using deep learning.

Specifically, provided is a method for predicting the activity of small Cas9, comprising: a step of designing a target sequence of small Cas9; and applying the target sequence designed by the step of designing above to the system for predicting the activity of small Cas9. The descriptions provided above are also applied to the method for predicting the activity of small Cas9.

Provided is a computer-readable recording medium having recorded thereon a program for causing a computer to execute a method for predicting the activity of small Cas9 using deep learning. The descriptions provided above are also applied to the computer-readable recording medium.

The program may implement the system for predicting the activity of small Cas9 or the method for predicting the activity of small Cas9 in a computer programming language.

The computer programming language capable of implementing the program may be Python, C, C++, Java, Fortran, Visual Basic, and the like, but is not limited thereto. The program may be stored in a recording medium such as a USB memory, a compact disc read only memory (CDROM), a hard disk, a magnetic diskette, or a similar medium or device, and may be connected to an internal or external network system. For example, a computer system may access a sequence database such as GenBank <http://www.ncbi.nlm.nih.gov/nucleotide> by using HTTP, HTTPS, or XML protocols, and search a nucleic acid sequence of a target gene and a regulatory region of the gene.

The program may be provided online or offline. The program may be provided in the form of a computer program stored in a recording medium to execute the system for predicting the activity of small Cas9 in combination with a computer-implemented electronic device.

Proviede is a method for providing information on small Cas9 and sgRNA that can specifically remove human single nucleotide mutations using deep learning. The descriptions provided above are also applied to the method for providing information on small Cas9 and sgRNA that can specifically remove human single nucleotide mutations.

Specifically, provided is a method for providing information on human single nucleotide mutations, comprising: a step of obtaining human single nucleotide variant data; a step of selecting data corresponding to pathogenic single nucleotide mutations among the human single nucleotide mutations; and a step of applying the selected data to the system for predicting the activity of small Cas9.

The step of applying the small Cas9 activity prediction system is to use a primary or secondary PAM existing at the mutant allele but not at the wild-type allele; or is to use a sgRNA perfectly matching the mutant allele but imperfectly coaching the wild-type allele.

The method for providing information small Cas9 and sgRNA that can specifically remove on human single nucleotide mutations may include the step of filtering out the combinations with on-target activity (activity at the mutant allele) lower than 10% and/or off-target activity (activity at the wild-type allele) higher than 2%, to identify efficient and mutant allele-specific small Cas9-sgRNA combinations for these mutations.

### BRIEF DESCRIPTION OF THE DRAWINGS

FIGS. 1A-1E show a massively parallel evaluation of the activities of the small Cas9s. FIG. 1A shows a schematic representation of the high-throughput experimental workflow; FIG. 1B shows a vector map of a sgRNA expression cassette paired with a barcode and the corresponding target sequence; FIG. 1C shows activities of the small Cas9s at randomly designed target sequences containing previously characterized PAM sequences. FIG. 1D shows a heatmap showing the Pearson correlation coefficients between the small Cas9-induced indel frequencies measured at the target sequences containing the same protospacers four days after transduction of the pairwise libraries into the small Cas9-expressing cells; FIG. 1E shows scatter plots showing the correlations of the four cases indicated as 1, 2, 3, and 4 in d of FIG. 1. PAM sequences used for the analyses are as follows: sRGN3.1, NNGGRT; SlugCas9, NNGGRT; SaCas9, NNGRRT; SauriCas9, NNGGRT; Sa-SlugCas9, NNGGRT; SaCas9*, NNGRRT; SaCas9-KKH, NNGRRT; eSaCas9, NNGRRT; efSaCas9, NNGRRT; SauriCas9-KKH, NNGGRT; SlugCas9-HF, NNGGRT; SaCas9-HF, NNGRRT; SaCas9-KKH-HF, NNGRRT; St1Cas9, NNRGAA; Nm1Cas9, NNNNGATT; enCjCas9, NNNNRYAC; CjCas9, NNNNRYAC; Nm2Cas9, NNNNCC.

FIG. 2 shows PAM compatibilities of the small Cas9s in human cells, heatmaps showing the average indel frequencies at target sequences with the indicated PAM sequences.

FIGS. 3A-3C show activities of small Cas9s and SpCas9 at mismatched target sequences. FIG. 3A shows heatmaps showing the specificities of small Cas9s and SpCas9 when there were one-base mismatches between the sgRNAs and target sequences with the primary or secondary PAM for each Cas9; FIG. 3B shows an effect of consecutive two-base transversion mismatches on the activities of small Cas9s and SpCas9. FIG. 3C shows a comparison of general activities(arithmetic mean of activities) and specificities(arithmetic mean of specificities) of the small Cas9s and SpCas9.

FIGS. 4A-4B show development and evaluation of computational models predicting the activities of the small Cas9s, evaluation of DeepSmallCas9 for the prediction of indel frequencies at matched (FIG. 4A) and mismatched (FIG. 4B) targets using hold-out test datasets of indel frequencies.

FIGS. 5A-5C show an allele-specific gene editing using small Cas9s. FIG. 5A shows two strategies for allele-specific disruption of dominant SNVs are shown using example sequences; (Figure discloses SEQ ID NOS 1, 2, 1, 3, 4, 5, 4, and 6, respectively in order of appearance); FIG. 5B shows a distribution of the predicted activities of small Cas9-sgRNA combinations designed to distinguish mutant alleles (on-targets=matched targets with primary or secondary PAMs) from wild-type alleles(off-targets=targets with inactive PAMs(top) or mismatched targets(bottom). FIG. 5C shows, of the 13,145 dominant SNVs in protein-coding sequences from the ClinVar database, the numbers of dominant SNVs that could be most efficiently and allele-specifically targeted with the indicated Cas9s; DeepSmallCas9-assisted selection of small Cas9-sgRNA combinations allowed efficient and allele-specific targeting of 10,844 of the 13,145 SNVs(Top pie chart), Random selection of small Cas9-sgRNA combinations resulted in efficient and allele-specific targeting for only 686 SNVs(Bottom pie chart). The box and whisker plot on the right shows the distribution of predicted activity in the wild-type and mutant alleles in these two cases above.

FIGS. 6A-6C show an expression of small Cas9s in HEK293T cells, FIG. 6A shows a schematic of the small Cas9-expressing cassette, FIG. 6B shows small Representative images of Western blotting used to measure the amount of small Cas9 proteins in HEK293T cells transduced 1553 with the lentiviral vectors encoding small Cas9s; FIG. 6C shows a relative levels of the small Cas9 proteins.

FIGS. 7A-7Q show the top 20 most important features associated with the 1571 activities of the small Cas9s.

FIGS. 8A-8B show PAM compatibilities of the small Cas9s in human cells, FIG. 8A shows heatmaps showing the average indel frequencies in the target sequences with the indicated PAM sequences; FIG. 8B shows a summary of the analyzed PAM compatibilities.

FIGS. 9A-9B show effects of sgRNA expression formats and scaffold sequences on the activities of the small Cas9s. FIG. 9A shows effects of sgRNA expression formats on the activities of the small Cas9s; FIG. 9B shows effects of sgRNA scaffold sequences on the activities of the small Cas9s.

FIGS. 10A-10D show sequences and secondary structures of sgRNA scaffolds for the small Cas9s. Figures disclose SEQ ID NOS 7-19, respectively, in order of appearance.

FIGS. 11A-11L show activities of sRGN3.1 and SlugCas9 at diverse potential off-target sequences, FIG. 11A and 11B shows a comparison of the activities of sRGN3,1 and SlugCas9 at different potential off-target sequences. FIG. 11C-11H shows heatmaps showing the average specificities of sRGN3.1 (FIGS. 11C, 11E, 11G) and SlugCas9 (FIGS. 11D, 11F, 11H) when there were 1-bp mismatches (FIGS. 11C, 11D), 1-nt RNA bulges (FIGS. 11E, 11F), or 1-nt DNA bulges (FIGS. 11G, 11H) between sgRNAs and target sequences with a primary or secondary PAM, FIGS. 11I-11L shows box plots showing the effects of deleted (FIGS. 11I, 11J) or inserted (FIGS. 11K, 11L) bases on the activities of sRGN3.1 (FIGS. 11I, 11K) and SlugCas9 (FIGS. 11J, 11L).

FIG. 12 shows the learning method of a set of deep learning-based models(DeepSmallCas9) that predict the activities of the small Cas9s at matched and mismatched target sequences. Figure discloses SEQ ID NOS 20, 21, 20, and 21, respectively, in order of appearance.

FIG. 13 shows a performance comparison of algorithms used to develop computational models that predict the activities of small Cas9s.

FIGS, 14A-14D show a comparison of the performance of DeepSmallCas9 with those of existing computational models predicting SaCas9 activity. FIG. 14A and 14B shows an evaluation of DeepSmallCas9 and “SaCas9 on-target rules” (ref. 48), an existing computational model 1668 predicting SaCas9 activities at matched target sequences; FIGS. 14C and 14D shows an evaluation of DeepSmallCas9 and “Model of SaCas9 specificity” (ref. 49), an existing computational model predicting SaCas9 activities at mismatched target sequences.

FIGS. 15A-15B show an evaluation and prediction of the activities of four small Cas9s in three different cell lines. FIG. 15A shows measured activities of four small Cas9s in three cell lines; FIG. 15B shows correlations between predicted and measured activities of four small Cas9s.

FIGS. 16A-16C show a computational prediction of preferred small Cas9s at targets with diverse PAM sequences. FIG. 16A shows a heatmap showing the most efficient Cas9 out of eight highly active small Cas9s, which include sRGN3.1, SlugCas9, SaCas9, SauriCas9, Sa-SlugCas9, SaCas9-KKH, eSaCas9, and efSaCas9, at target sequences with a given PAM sequence; FIG. 16B shows a pie chart showing the number of PAM sequences that could be most efficiently targeted with each Cas9 with an average activity higher than 10%; FIG. 16C shows a bar graph showing the numbers of efficiently targetable PAM sequences out of 4,096 (=4⁶) PAMs for each Cas9 with an average activity higher than 10%.

FIGS. 17A-17E show SlugCas9-, SaCas9-KKH-, SlugCas9-HF, Sa-SlugCas9-, or efSaCas9-directed targeting of dominant single-nucleotide variants with or without using DeepSmallCas9 to select sgRNAs, pie charts showing the fraction of the dominant single-nucleotide variants in protein-coding sequences in the ClinVar database (ref. 110 and 111) that can be edited using SlugCas9 (FIG. 17A), SaCas9-KKH (FIG. 17B), SlugCas9-HF (FIG. 17C), Sa-SlugCas9 (FIG. 17D), or efSaCas9 (FIG. 17E) in an efficient and allele-specific manner (on-target activity higher than 10% and off-target activity lower than 2%).

FIG. 18 shows an allele-specific gene editing using small Cas9s and SpCas9.

FIG. 19 shows a comparison of the sizes of Cas nuclease proteins

FIG. 20 shows a generation of plasmid libraries. Figure discloses SEQ ID NOS 22-25, respectively, in order of appearance.

FIGS. 21A-21B show correlations between indel frequencies of independently transduced replicates.

FIG. 22 shows an engineering the SaCas9 scaffold; SaCas9 scaffolds 4 and 5 were derived from SaCas9 scaffold 3; the modified regions are shown in red. Figure discloses SEQ ID NOS 9 and 26-27, respectively, in order of appearance.

FIG. 23 shows an Indel frequencies measured at matched target sequences.

FIG. 24 shows an effects of one-base mismatches on the activities of small Cas9s and SpCas9.

FIG. 25 shows an effect of mismatch types on the activities of small Cas9s and SpCas9.

FIG. 26 shows an effects of consecutive two-base transversion mismatches on the activities of small Cas9s and SpCas9.

FIG. 27 shows activities of the small Cas9s and SpCas9 at randomly designed target sequences containing the primary PAM sequences.

FIGS. 28A-28B show an evaluation of computational models predicting the activities of the sRGN3.1 and SlugCas9 at matched targets (FIG. 28A) and targets containing mismatches, insertions, or deletions (FIG. 28B).

FIG. 29 shows a pathogenic mutant allele-specific gene editing using SlugCas9, SaCas9-KKH, and SlugCas9.

FIG. 30 shows an evaluation of DeepSpCas9-v2 DeepSpCas9-v2, for the prediction of indel frequencies at matched(left) and mismatched(right) targets, hold-out test datasets of indel frequencies were used.

FIG. 31 shows a schematic of sgRNA selections by the web tool.

### DETAILED DESCRIPTION

The disclosure will be described in more detail with reference to the following embodiments. However, the embodiments are for illustrative purposes only and the scope of the disclosure is not limited thereto.

### Embodiment 1. Preparation of Materials

### Embodiment 1-1. Construction of Plasmids Encoding Cas9s

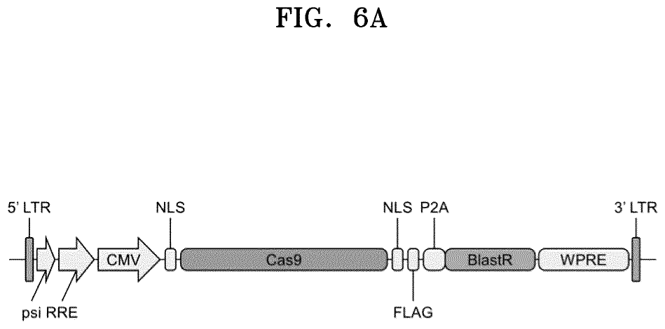

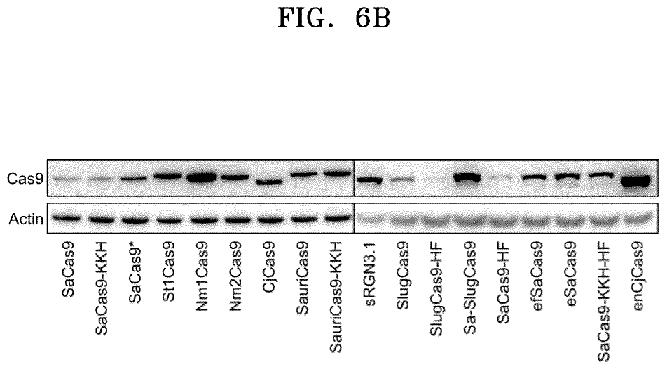

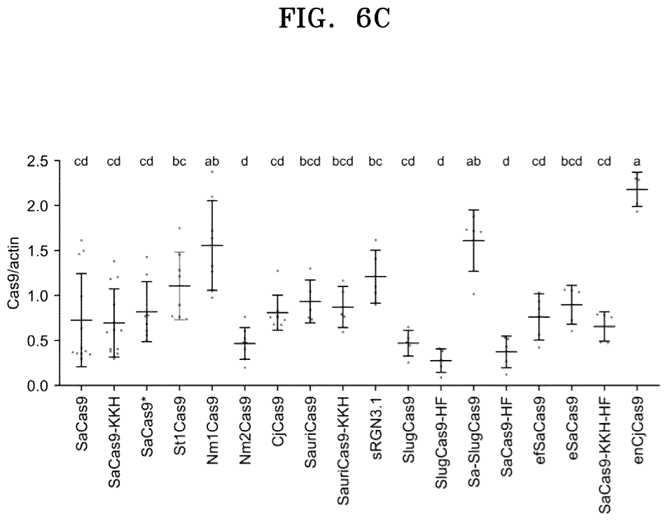

To construct the small Cas9-or SpCas9-encoding plasmids, the ABE7.10-encoding sequence was removed from Lenti-ABE-Blast89and replaced with the Cas9-encoding sequences from MSP2283 (Addgene, #70702), MSP1830 (Addgene, #70708), pCAG-CFP-SaCas9-HF (without sgRNA)(Addgene, #134470), pUC57-Mini-SaCas9*, pUC57-Mini-SauriCas9, pUC57-Mini-SauriCas9-KKH, pUC57-Mini-St1Cas9, pUC57-Mini-Nm 1Cas9, pUC57-Mini-Nm2Cas9, pUC57-Mini-CjCas9, pTwist-Kan-High Copy-sRGN3.1, pTwist-Kan-High Copy-SlugCas9, pTwist-Kan-High Copy-SlugCas9-HF, pTwist-Kan-High Copy-Sa-SlugCas9,or lentiCas9-Blast (Addgene, #52962); Cas9 sequences encoded in pUC57-Mini or pTwist-Kan-High Copy plasmids were GenScript codon-optimized. The resulting plasmids are referred to as pLenti6.3-SaCas9-BlastR, pLenti6.3-SaCas9-KKH-BlastR, pLenti6.3-SaCas9-HF-BlastR, pLenti6.3-SaCas*-BlastR, pLenti6.3-SauriCas9-BlastR, pLenti6.3-SauriCas9-KKH-BlastR, pLenti6.3-St1Cas9-BlastR, pLenti6.3-Nm1Cas9-BlastR, pLenti6.3-Nm2Cas9-BlastR, pLenti6.3-CjCas9-BlastR, pLenti6.3-sRGN3.1-BlastR, pLenti6.3-SlugCas9-BlastR, pLenti6.3-SlugCas9-HF, pLenti6.3-Sa-SlugCas9-BlastR, and pLenti6.3-SpCas9-BlastR, respectively. pLenti6.3-efSaCas9-BlastR and pLenti6.3-eSaCas9-BlastR were derived from pLenti6.3-SaCas9-BlastR, pLenti6.3-SaCas9-KKH-HF-BlastR was derived from pLenti6.3-SaCas9-KKH-BlastR, and pLenti6.3-enCjCas9-BlastR was derived from pLenti6.3-CjCas9-BlastR by introducing mutations. The small Cas9-expressing cassettes are shown in FIG. 6.

### Embodiment 1-2. Oligonucleotide Library Design

A total offiveoligonucleotide pools were array synthesized by Twist Bioscience. Each oligonucleotide contained a guide sequence, a BsmBl restriction site, a variable stuffer sequence, another BsmBl restriction site, a barcode, a second variable stuffer sequence, and the corresponding target sequence with a PAM sequence.

Oligonucleotide pool A, consisting of 77,712 pairs of guide sequences and the corresponding target sequences, was designed to evaluate activities at matched and mismatched target sequences and the PAM compatibilities of the small Cas9s. Five PAM sequences were used: NNGRRT (Staphylococcus-derived Cas9s), NNRGAA (St1Cas9), NNNNGATT (Nm1Cas9), NNNNCC (Nm2Cas9), and NNNNRYAC (Campylobacter jejuni-derived Cas9s). The target sequences included 50,000 (10,000 randomly designed protospacers without any restriction in GC content×5 PAM sequences) and 2,370 (474 randomly designed protospacers having low (<24%) or high (>76%) GC content×5 PAM sequences) target sequences. In addition, 11,520 target sequences were designed using previously used protospacer sequences: 2,400 targets (30 protospacers×80 PAMs(64 NNNNNTN+16 NNGRRNN, evaluated nucleotides in the PAM are underlined in bold)) for Staphylococcus-derived Cas9s, 2,400 targets(30 protospacers×80 PAMs (64 NNNNNAN+16 NNRGANN)) for St1Cas9, 2,400 targets(30 protospacers×80 PAMs (64 NNNNNNNTN+16 NNNNGATNN)) for Nm1Cas9, 1,920 targets(30 protospacers×64 PAMs (NNNNNTN)) for Nm2Cas9, and 2,400 targets(30 protospacers×80 PAMs (64 NNNNNNNCN+16 NNNNRYANN)) for Campylobacter jejuni-derived Cas9s. We also included 12,810 targets with mismatch(es) in the protospacer sequences using previously tested protospacers which include 2,580 targets (30 guide sequences×(63 targets with one-base mismatches+20 targets with consecutive two-base transversion mismatches+1 perfectly matched target×3 different barcodes)) for Staphylococcus-derived Cas9s, 2,340 targets (30 guide sequences×(57 targets with one-base mismatches+18 targets with consecutive two-base transversion mismatches+1 perfectly matched target×3 different barcodes)) for St1Cas9, 2,820 targets (30 guide sequences×(69 targets with one-base mismatches+22 targets with consecutive two-base transversion mismatches+1 perfectly matched target×3 different barcodes)) for Nm1Cas9, 2,820 targets (30guide sequences×(69 targets with one-base mismatches+22 targets with consecutive two-base transversion mismatches+1 perfectly matched target×3 unique barcodes)) for Nm2Cas9, and 2,700 targets (30 guide sequences×(66 targets with one-base mismatches+21 targets with consecutive two-base transversion mismatches+1 perfectly matched target×3 different barcodes)) for Campylobacter jejuni-derived Cas9s. Lastly, 687 sequences were included, but excluded from the analysis. Taken together, 50,000 (=10,000×5)+2,370 (=474×5)+11,520 (+2,400+2,400+2,400 +1,920+2,400)+13,260 (=2,580+2,340+2,820 +2,820+2,700)+687−125 (containing an additional BsmBl site)=77,712 pairs were included in oligonucleotide pool A.A 5′ guanine was held constant for every guide sequence except the 687 sequences excluded from the analysis. Also, one of five different reverse primer binding sequences was included in the oligonucleotides for selective amplification of sequences for the generation of five individual plasmid libraries.

Oligonucleotide pool B, consisting of 55,191 pairs of guide and corresponding target sequences, was used to evaluate the activity at matched targets and to characterize the PAM specificity of SaCas9 and SaCas9-KKH, This pool included 19,583 randomly designed target sequences without any restriction in GC content followed by an NNGRR PAM, 1,941 randomly designed sequences having low (<20%) or high (>80%) GC content followed by an NNGRR PAM, 9891 targets with an NNGRR PAM obtained from human coding sequences, and 16,892 targets (44 protospacers×385 PAMs (64 NNNAGTCA+256 CTNNNNAG+64 CTGAGNNN+1 NNGRRTNN)-48 targets containing an additional BsmBl site) designed with previously studied protospacer sequences. Additionally, 6,884 sequences were included, but not analyzed in this study. A 5′ guanine was held constant for every guide sequence except the 6,884 sequences excluded from the analysis.

Oligonucleotide pool C, consisting of 11,525 pairs of guide and corresponding target sequences, was designed to determine the optimal spacer length, U6-driven transcription format, and scaffold sequence for each small Cas9.In this pool, 2,090 sequences (418 randomly designed protospacers followed by NNGRRT×(1G/g-N20+1G/g-N21+1G/g-N22+1 A/a-N21+1 tRNA-N21)) for SaCas9 and SauriCas9, 2508 sequences (418 randomly designed protospacers followed by NNAGAA×(1 G/g-N18+2G/g-N19+1 G/g-N20+1 A/a-N19+1 tRNA-N19)) for St1Cas9, 2090 sequences (418 randomly designed protospacers followed by NNNNGATT×(1 G/g-N22+1 G/g-N23+1G/g-N24+1 A/a-N23+1 tRNA-N23)) for Nm1Cas9, 2090 sequences (418 randomly designed protospacers followed by NNNNCC×(1 G/g-N21+1 G/g-N22+1 G/g-N23+1 A/a-N22+1 tRNA-N22)) for Nm2Cas9, and 2090 sequences (418 randomly designed protospacers followed by NNNNACAC×(1 G/g-N21 +1 G/g-N22+1 G/g-N23+1 A/a-N22+1 tRNA-N22)) for CjCas9 were included. Another 657 sequences were included, but excluded from the analysis. For the generation of eleven plasmid libraries from the oligonucleotide pool, one of two different forward primer binding sequences and one of eleven different reverse primer binding sequences were included in the oligonucleotides.

Oligonucleotide pool D, consisting of 35,990 pairs of guide and corresponding target sequences, was designed to evaluate the activities of Staphylococcus-derived Cas9s at target sequences with mismatch(es) or with a DNA or RNA bulge between the spacer and protospacer sequences and to validate mutant allele-specific disruption by small Cas9 variants. We designed 10,243 pairs with an NNGRRT PAM (75 guide sequences used in oligonucleotide pool A×(63 targets with one-base mismatches+20 targets with two-base mismatches+10 targets with three-base mismatches+21 targets with one-base deletions+20 targets with one-base insertions+1 perfectly matched target×3 different barcodes)−32 pairs containing an additional BsmBl site) for sRGN3.1 and SlugCas9; the targets with two-base mismatches, three-base mismatches, or one-base insertions were randomly selected from all such possible targets. From the ClinVar database, we also included 182 pairs (=91 guide RNA sequences×(1 mutant target sequence containing a dominant pathogenic mutation+1 corresponding wild-type target sequence)) for SlugCas9 and SlugCas9-HF and 66 pairs (=33 guide RNA sequences×(1 mutant target sequence containing a dominant pathogenic mutation in the PAM sequence+1 corresponding wild-type target sequence)) for SaCas9-KKH; among these pairs, 114 for SlugCas9, 78 for SlugCas9-HF, and 40 for SaCas9-KKH were predicted to be efficient (predicted activity at the mutant allele >10%) and mutant allele-specific (predicted activity at the wild-type allele <2%) by DeepSmallCas9 and were used for the analysis. Additional 25,499 sequences were included, but not analyzed in this study. A 5′ guanine was held constant for every guide sequence. In addition, one of three different reverse primer binding sequences was included in the oligonucleotides for selective amplification of sequences for the generation of three individual plasmid libraries.

Oligonucleotide pool E, consisting of 5,402 pairs of guide and corresponding target sequences, was designed to evaluate the activities of SpCas9 at matched target sequences. This pool included 5,210 target sequences generated by combining 5,210 randomly designed protospacers used in oligonucleotide pool A with an NGG PAM; 192 target sequences included in this pool were not tested in the current study. A 5′ guanine was held constant for every guide sequence.

### Embodiment 1-3. Plasmid Library Preparation

To prepare the plasmid libraries containing sgRNA-encoding and corresponding target sequences, the cloning process was as previously described with minor changes.

### Step 1: Generation of the First Plasmid Library Containing Pairs of Guide-Encoding Sequences and Corresponding Target Sequences

The Lenti-gRNA-euro plasmid (Addgene, #84752) and Lenti-tRNAGln-gRNA-Puro plasmid were linearized with BsmBl-v2 restriction enzyme (NEB) at 55° C. for 1.5 h, after which they were treated with Quick CIP (NEB) at 37° C. for 10 min. The linearized and dephosphorylated plasmids were separated on a 0.8% agarose gel and purified using a QIAquick Gel Extraction Kit (Qiagen).

The pooled oligonucleotides were PCR-amplified using Q5 High-Fidelity DNA Polymerase (NEB). The PCR products were separated on a 4% agarose gel and purified using a QIAquick Gel Extraction Kit (Qiagen).

The purified amplicons and the linearized Lenti-gRNA-Puro or Lenti-tRNA^{Gln-} gRNA-Puro plasmid were assembled using an NEBuilder HiFi DNA Assembly Kit (NEB) at 50° C. for 1 h. After incubation, the products were precipitated using isopropanol as previously described and electroporated into Endura™ ElectroCompetent Cells (Lucigen) using a MicroPulser (Bio-Rad). The treated cells were then spread on Luria-Bertani agar plates containing 50 μg ml⁻¹ carbenicillin and incubated at 37° C. for 16 h. Small fractions (0.01 μl, 0.1 μl, 1 μl) of transformed cells were spread on separate plates to calculate the library coverage. Colonies were harvested and the plasmids were purified using a Plasmid Maxi Kit (Qiagen). The cloning efficiency was evaluated with more than 10 individually isolated plasmids by Sanger sequencing.

### Step 2: Generation of the Second Plasmid Library

In preparation for inserting the sgRNA scaffold sequence, the first plasmid library described above was digested with BsmBl-v2 restriction enzyme (NEB) at 55° C. for 3-15 h, after which it was treated with Quick CIP (NEB) at 37° C. for 10 min. The linearized and dephosphorylated plasmids were separated on a 0.8% agarose gel and purified using a QlAquick Gel Extraction Kit(Qiagen).

The sgRNA scaffold sequences were PCR-amplified from plasmids obtained from Addgene or synthesized oligonucleotides (IDT) with Q5 High-Fidelity DNA polymerase (NEB) using primers containing BsmBl restriction sites, after which the sequences were cloned into pCR-Blunt II-TOPO (Invitrogen). pCR-Blunt II-TOPO containing St1Cas9 scaffold 2 was generated by removing one nucleotide from pCR-Blunt II-TOPO containing St1Cas9 scaffold 1. The plasmids were digested with BsmBl-v2 restriction enzyme (NEB) at 55° C. for 2-7.5 h and gel purified using a QIAquick Gel Extraction Kit (Qiagen).

The digested first plasmid library (100 ng) and sgRNA scaffold (10-40 ng) were ligated using T4 DNA Ligase (NEB) at 25° C. for 1 h. After ligation, the enzyme was heat inactivated at 65° C. for 10 min. The products were precipitated using isopropanol as previously described and electroporated into Endura™ ElectroCompetent Cells (Lucigen) using a MicroPulser (Bio-Rad). The treated cells were then spread on Luria-Bertani agar plates containing 50 μg ml⁻¹ carbenicillin and incubated at 37° C. for 16 h. Small fractions (0.01 μl, 0.1 μl, 1 μl) of transformed cells were spread on separate plates to calculate the library coverage. Colonies were harvested and the plasmids were purified using a Plasmid Maxi Kit (Qiagen). The cloning efficiency was evaluated with more than 10 individually isolated plasmids by Sanger sequencing.

### Embodiment 1-4. Lentivirus Production

HEK293T cells (ATCC) were maintained in Dulbecco's Modified Eagle Medium (Gibco) containing 10% fetal bovine serum (Gibco). For virus production, HEK293T cells were seeded in 150-mm dishes at a density of 3×10⁷ cells per dish 1 d before transfection. On the day of transfection, the cultures were replenished with fresh medium. The lentiviral transfer plasmid (6.56 pmol), psPAX2 (Addgene, #12260, 5.2 pmol), and pMD2.G (Addgene, #12259, 2.88 pmol) were mixed with polyethylenimine (Polyplus-transfection) and incubated for 20-30 min at room temperature. After incubation, the mixture was added dropwise to the HEK293T cells. At 18 h after transfection, the culture medium was removed and replaced with fresh medium. The supernatant containing virus particles was collected at 48 h and 72 h post-transfection. Individual harvests were pooled, filtered with a Millex-HV 0.45-μm low protein-binding membrane (Millipore), and stored at −80 ° C. in small aliquots.

To determine the lentiviral titer, an aliquot containing lentivirus was serially diluted and transduced into HEK293T cells in the presence of 10 μg polybrene. Both virus-treated and untreated cells were maintained in the presence of 2 μg ml⁻¹ puromycin (Gibco) or 20 μg ml⁻¹ blasticidin S (InvivoGen) until no viable cells remained in the untreated cell population. The number of cells that survived in the virus-treated population was counted to provide an estimate of the functional titer of the virus as previously described.

### Embodiment 1-5. Generation of the Small Cas9-Expressing Cell Lines

To generate cell lines with stable small Cas9 expression, HEK293T, DLD-1, or HCT116 cells were transduced with each Cas9-encoding lentivirus at an MOI of 0.1 in the presence of 10 μg ml⁻¹ polybrene. Cells were selected with 20 μg ml₋₁ blasticidin S (InvivoGen) starting from the day after transduction and this selection was continued for at least 11 days before the transduction with the lentiviral library.

Embodiment 1-6. Lentiviral Library Transduction into the Small Cas9-Expressing Cell Lines

Cas9-expressing cells were seeded and each cell line was infected with the lentiviral library at an MOI of 0.4 in the presence of 10 μg ml⁻¹ polybrene. Infected cells were selected with puromycin (Gibco) starting 24 h after transduction and harvested four and/or seven days after transduction of the library.

### Embodiment 1-7. Determination of Indel Frequencies at Endogenous Loci

To evaluate SaCas9 scaffod 3 and the engineered SaCas9 scaffolds (SaCas9 scaffolds 4 and 5), HEK293T cells were seeded into 12-well plates at a concentration of 2.5×10⁵ cells per well. After 24 h, the cells were transfected with 500 ng of pLenti6.3-SaCas9-BlastR and 1,500 ng of Lenti-gRNA-Puro (Addgene, #84752) encoding sgRNA using Lipofectamine 2000 (lnvitrogen). Transfected cells were selected with blastcidin S (InvivoGen) and puromycin (Gibco) starting 24 h after transfection and harvested three days after transfection,

### Embodiment 2. Experimental Method and Outcome Measurement

### Embodiment 2-1. Deep Sequencing

Genomic DNA was isolated from cell pellets with a Wizard Genomic DNA Purification Kit (Promega) and amplified by two-step PCR. In the first FOR, the integrated target sequences including barcodes were amplified with 2× Taq FOR Smart Mix (Solgent) using the genomic DNA as template; the total amount of genomic DNA used for amplification represented more than 1000× coverage of the library, assuming 10 μg of genomic DNA per 10⁶ HEK293T cells. The products were combined and purified using a MEGAquick-spin Plus Total Fragment DNA Purification Kit (iNtRON Biotechnology). The purified products were separated on a 4% agarose gel and purified with a MEGAquick-spin Plus Total Fragment DNA purification Kit (iNtRON Biotechnology). For the second PCR, primers containing both Illumina adaptor and barcode sequences were used to amplify the purified products from the first PCR. The resulting amplicons were pooled, purified using a MEGAquick-spin Plus Total Fragment DNA purification Kit (iNtRON Biotechnology), and sequenced on a HiSeq 2500 (Illumina), a MiniSeq (Illumina); or a NovaSeq 6000 (Illumina).

In the case of cells transfected with SaCas9- and sgRNA-encoding plasmids, the cells were lysed by incubating for 60 min at 37° C., 40 min at 55° C. 30 min at 85° 0 C., and 10 min at 95° C. in a lysis buffer (10 mM Tris-HCl pH 8.0, 0.05% SDS, and 20 μg ml⁻¹ proteinase K). The endogenous loci were amplified from the cell lysates with 2× Taq PCR 1005 Smart Mix (Solgent) and then the amplicons were further amplified with the primers containing both Illumina adaptor and barcode sequences. The resulting amplicons were separated on a 4% agarose gel, purified with a MEGAquick-spin Plus Total Fragment DNA purification Kit (iNtRON Biotechnology), and sequenced on a MiniSeq (Illumina).

Embodiment 2-2. Determination of the Frequency of Shuffling between sgRNA-Encoding and Barcode-Target Sequences

Genomic DNA extracted from cells transduced with library 6 was amplified with LongAmp Tag 2× Master Mix (NEB) and prepared for deep sequencing through two PCR steps. To measure the pre-existing and PCR-induced shuffling frequency, DNA from plasmid library 6 was prepared and sequenced using the same steps. The first PCR was conducted using genomic DNA containing 1,000 or 100,000 copies of the lentiviral library, assuming 10 pg of genomic DNA per cell, or 1,000 or 100,000 copies of plasmid library. The products were precipitated using isopropanol and gel purified using a QIAquick Gel Extraction Kit (Qiagen). In the second PCR, primers containing both Illumina adaptor and barcode sequences were used to amplify 100 pg of the purified products from the first PCR. The products were purified using a QIAquick Gel Extraction Kit (Qiagen) and sequenced on a NovaSeq 6000 (Illumina). The frequency of shuffling during lentiviral packaging was calculated by subtracting the pre-existing and PCR-induced shuffling frequency from the observed shuffling frequency

### Embodiment 2-3. Analysis of Indel Frequencies

Previously developed Python scripts (CRISPResso2)120 were modified and used for the analysis of deep sequencing data (see Embodiment 2-13. Code availability). Guide-target pairs were individually identified with a 22-nt sequence (TTTG+barcode). Changes in the sequence in the 8-nt window (4 nucleotides on either side of the cleavage site) were counted as nuclease-induced indels. Array synthesis and PCR amplification can also result in indels. Such background indel frequencies were eliminated by subtracting them from measured indel frequencies using the Equation 1 below:

$$Indel frequency ( \% ) = \frac{\begin{matrix}Indel read counts - \\ ( Total read \times Background indel frequency )\end{matrix}}{\begin{matrix}Total read counts - \\ ( Total read \times Background indel frequency )\end{matrix}} \times 100 \qquad [ Equation 1 ]$$

The read counts of replicates 1 and 2 were combined for analyses and the data were filtered to increase the accuracy of the analysis. Guide-target pairs with <100 combined (replicates 1 and 2) read counts or >8% background indel frequencies were excluded from the analyses as we previously described.

When cells were transfected with plasmids encoding small Cas9s and sgRNAs, the indel frequencies were analyzed using previously developed Python scripts (CRISPResso2) with the following parameters: minimum homology score with the amplicon to be aligned=70, 8-nt window for quantification (4 nucleotides on either side of the cleavage site), substitutions ignored, minimum average read quality score (phred33)=10, and minimum single base pair score quality score (phred33)=10. Background indel frequencies measured in untransfected cells were subtracted from indel frequencies for analysis.

### Embodiment 2-4. Western Blotting

To measure the expression level of FLAG-tagged Cas9, the Cas9-expressing cells were lysed by incubation in a lysis buffer (20 mM HEPES, 150 mM NaCl, 1% NP-40, 0.25% sodium deoxycholate, and 10% glycerol) containing a 1:100 dilution of protease inhibitor cocktail (Cell Signaling Technology) for 20 min on ice and then centrifuged at 13,000 g for 15 min at 4° C. The total protein concentration was determined using a Bradford protein assay kit (Pierce). Proteins (30 or 60 μg) were loaded into and separated in 4-12% Bis-Tris gels in 1× NuPage MES SDS running buffer (Invitrogen) at 120 V for 2 h. Thereafter, proteins were transferred onto a 0.45 μm Invitrolon polyvinylidene difluoride membrane (Invitrogen) in 1× NuPage Transfer buffer containing 10% (volivol) methanol using an XCell II Blot Module (Invitrogen) for 1 h on ice. The membranes were blocked with 5% bovine serum albumin (BSA) in 1× Tris-buffered saline with 1% Tween 20 (TBST) for 1 h and then incubated with the following primary antibodies: anti-FLAG M2 (Sigma, cat. no. F1804-50UG) at 1:1,000 dilution and anti-β-actin C4 (Santa Cruz Biotechnology, cat. no. sc-47778) at 1:2,000 dilution in 1× TBST containing 5% BSA overnight at 4° C. The next day, the blots were washed three times with 1× TBST and incubated for 1 h with horseradish peroxidase-conjugated goat anti-mouse IgG secondary antibodies (Santa Cruz Biotechnology, cat. no. sc-516102) at 1:3,000 dilution in 1× TBST containing 3% BSA at room temperature. To develop the blots, West-Q Pico ECL. Solution (GeneDEPOT), the ImageQuant LAS-4000 digital imaging system (GE Healthcare), and the Amersham ImageQuant 800 system (Cytiva) were used.

Embodiment 2-5. Generation of the Training and Test Datasets used for the Development and Evaluation of Computational Models

The guide and wide target sequences (each consisting of a 4-nt 5′ neighboring sequence, a protospacer, a PAM, and a 3-nt 3′ neighboring sequence) and indel frequencies measured four days after transduction of the libraries 1-6 were used for the generation of the datasets. In this process, the guide and mismatched target pairs designed from the matched targets with an average indel frequency of less than 2% were excluded. The remaining data were randomly split into the training (90%) and test (10%) datasets and the few pairs shared by both datasets were removed from the test datasets for a fair evaluation of the models.

### Embodiment 2-6. Conventional Machine Learning-Based Model Training

Seven models were trained based on the following conventional machine learning algorithms: extreme gradient boosting (XGBoost), gradient-boosted regression trees (Boosted RT), random forest (RF), L1-regularized linear regression (Lasso) ; L2-regularized linear regression (Ridge) ; L1 and L2-regularized linear regression (Elastic Net), and support vector machine (SVM). We used the XGBoost Python package (version 1.3.3) for XGBoost and scikit-learn (version 0.23.2) for all the other models. The numbers of features extracted from the guide and wide target sequences were as follows: sRGN3,1, n=907; SlugCas9, n=907; SaCas9, n=947; SauriCas9, n=907; Sa-SlugCas9, n=907; SaCas9-KKH, n=947; eSaCas9 ; n=947; efSaCas9, n=947; SauriCas9-KKH, n=907; SlugCas9-HF, n=907; SaCas9-HF, n=947; SaCas9-KKH-HF, n=947; St1Cas9, n=883; Nm1Cas9, n=1,051; enCjCas9, n=1,019; CjCas9, n=1,019; Nm2Cas9 ; n=1,031. The features included all possible position-independent and position-dependent nucleotides and dinucleotides in the wide target sequence, melting temperatures calculated from seven different regions in the wide target sequence, the numbers of G or C nucleotides in the spacer and protospacer, the MFEs of the spacer and the sgRNA (spacer+scaffold) and the mismatch positions and types between the guide and protospacer sequences. To calculate the melting temperature, a program (<https://biopython.org/docs/1.74/api/Bio.SeqUtils.MeltingTemp.html>) was used with a default setting that does not consider the nuclear milieu within the cell; the MFE was calculated using Vienna RNASubOpt. For model selection among the regularization parameters and hyperparameter configurations, we conducted five-fold cross-validation. For conventional machine learning algorithms such as XGBoost, Boosted RT, RF, Lasso, Rigde, Elastic Net, and SVM, we searched over 144 models for each algorithm using the hyperparameters previously described.

### Embodiment 2-7. Evaluation of Feature Importance

Feature importance was interpreted using the Tree SHAP method. We extracted features from guide and wide target sequences and trained XGBoost models with the best hyperparameter configurations determined from five-fold cross-validation as described above. Each feature from the trained XGBoost models then received a per-sample importance score, which indicates the impact of the feature on the base value in the model output and is determined using a game theoretic Shapley value for optimal credit allocation. As a summary of feature importance in our models, we provide SHAP value distributions for the whole data set or the mean absolute value.

### Embodiment 2-8. Development of DeepSmallCas9

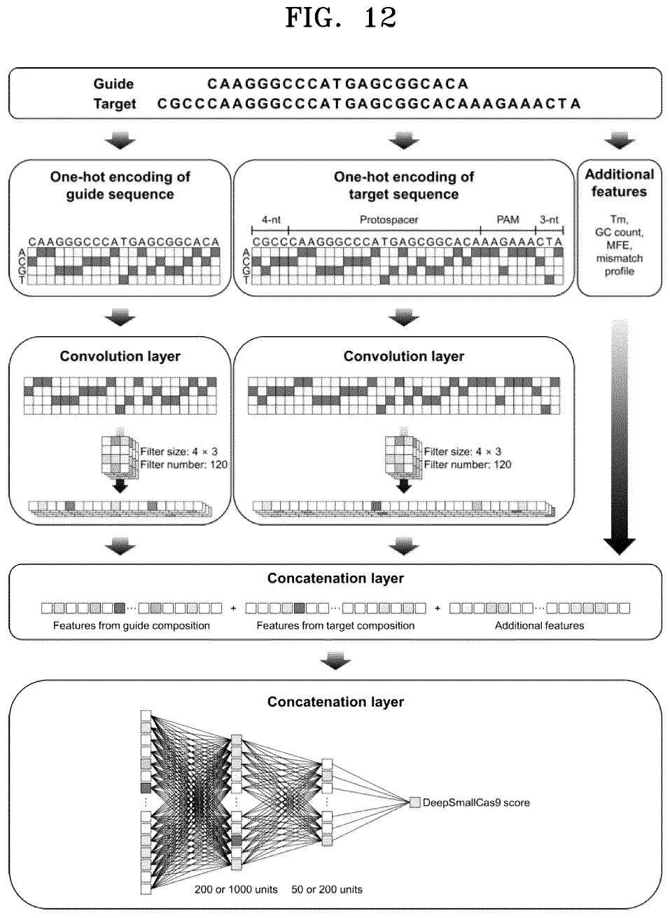

DeepSmallCas9 is a set of deep learning-based computational models that predict the activities of the small Cas9s at both matched and mismatched target sequences (in the case of sRGN3.1 and SlugCas9, the activities at targets containing insertions or deletions can also be predicted). To generate DeepSmallCas9, the guide sequence, the wide target sequence, additional calculated features (melting temperatures calculated from seven different regions in the wide target sequence, the numbers of G or C nucleotides in the spacer and protospacer, the MFEs of the spacer and the sgRNA (spacer+scaffold) and the mismatch positions and types between guide and protospacer sequences), and the measured indel frequency were used to generate the training datasets. During the model selection phase, these training data were used for five-fold cross validation. Input sequences were converted into a four-dimensional binary matrix using one-hot encoding (FIG. 12). DeepSmallCas9 was developed using a convolutional layer and two fully connected layers. The convolution layer obtained an embedding vector from the guide and wide target sequences using 120 filters with 3-nt windows. Then, the embedding vector was concatenated with the additional calculated features. To maintain local information, the pooling layer was excluded as the deep reinforcement learning algorithm was implemented. We next used the two fully connected layers: one with 200 or 1,000 units and the other with 50 or 200 units using the rectified linear unit as an activation function. The regression output layer linearly transformed the outputs and calculated the prediction score for the activity of the small Cas9s. We tested 12 different models (hyperparameters; number of filters (30, 60, 120) and units (200 or 1,000 and 50 or 200) for the convolutional layer and fully connected layers, respectively) and then selected the model that resulted in the highest Spearman correlation coefficients between the experimentally determined and predicted activity levels during the five-fold cross-validation. To avoid overfitting, dropout was used at a rate of 0.3. The mean squared error was used as the objective function, and an Adam optimizer was used with a learning rate of 10⁻³. DeepSmallCas9 was implemented with TensorFlow.

### Embodiment 2-9. Development of DeepSpCas9-v2

DeepSpCas9-v2 is a deep learning-based computational model that predicts the activities of SpCas9 at both matched and mismatched target sequences. For the generation of DeepSpCas9-v2, the indel frequency datasets obtained in this study were used and the method used for the generation of DeepSmallCas9 was applied.

Embodiment 2-10. Obtaining Suggestions for which Small Cas9 and Guide Sequence to use for the Disruption of Dominant Single-Nucleotide Variants in Coding Sequences

Of 774,186 mutations in the ClinVar database (downloaded on 4 Sep. 2020) 13,145 dominant SNVs in protein-coding sequences were sorted using protein-coding sequence annotations from Matched Annotation from NCBI and EMBL-EBI (MANE) Select v.0.95 (<https://ftp.ncbi.nlm.nih.gov/refseg/MANE/MANE_human/release_0.95/>). sgRNAs that can distinguish mutant and wild-type alleles were selected as described below. All possible sgRNAs were extracted if primary or secondary PAM sequences were found in the mutant sequence but not in the wild-type sequence as described previously, or if the sgRNAs could recognize the mutant sequence and had at least one nucleotide mismatch with the wild-type sequence. For DeepSmallCas9-assisted selection of small Cas9-sgRNA combinations, we calculated predicted activities at the mutant alleles and the corresponding wild-type alleles using DeepSmallCas9. Based on the predicted activities at the mutant and wild-type alleles, inefficient (predicted activity at the mutant allele lower than 10%) and/or nonspecific (predicted activity at the wild-type allele higher than 2%) sgRNAs were filtered out and the remaining sgRNAs were ranked by predicted activities at the mutant allele (in descending order) and at the wild-type allele (in ascending order). The ranks were combined for each sgRNA sequence and a sgRNA with the lowest combined rank was chosen, When multiple sgRNAs for the same mutation received the same lowest combined rank value, the sgRNA with the highest activity at the mutant allele was selected. In the case of random selection or rational selection based on the location of the mutation (i.e., Cas9-sgRNA combinations targeting the mutation in a PAM region are the most preferred and the combinations targeting the mutation in PAM-adjacent and PAM-distal protospacer regions are the second most and least preferred, respectively), a small Cas9-sgRNA combination for each mutation was selected randomly or rationally and then the activities of the selected combinations were predicted using DeepSmallCas9 to compare with DeepSmallCas9-assisted selection.

Embodiment 2-11. Development of a Web Tool to Design sgRNAs for the Small Cas9s

We generated a web tool (<http://deepcrispr.info/DeepSmallCas9>) to design sgRNAs for experiments using the small Cas9s by combining deep learning-based models that predict activities at matched and mismatched targets (DeepSmallCas9) and Cas-OFFinder, an algorithm that searches for potential off-target sites of Cas9s. GRCh38.p13 v.104 and GRCm39 v.104 from Ensembl were used as reference genomes and Matched Annotation from 1185 NCBI and EMBL-EBI (MANE) Select v.0.95 (<https://ftp.ncbi.nlm.nih.gov/refseq/MANE/MANE_human/release_0.95/>) and RefSeq Select (downloaded on 18 Aug. 2021), which both provide a representative transcript per gene, were respectively used for annotation of human and mouse protein-coding sequences. The web tool process is as follows. (1) Candidate targets are found using primary PAMs (if an input is a gene, candidate targets for which Cas9 cleavage sites are in the protein-coding sequence of the gene are found) and the activities at these targets are calculated using DeepSmallCas9. (2) Genome-wide mismatched targets as potential off-targets are found using Cas-OFFinder (webtool users are asked to select the maximum number of mismatched bases, with a default value of three) and the activities at these targets are calculated using DeepSmallCas9. (3) The sum of activities at mismatched targets is obtained for each candidate sgRNA. (4) The on-target activity and the sum of the off-target activities for each sgRNA are ranked in descending and ascending order, respectively. These ranks are combined as similarly conducted for SpCas9 and the sgRNA with the lowest combined rank is the most highly recommended. When multiple sgRNAs receive the same lowest combined rank value, they are listed in the order of high to low on-target activity.

### Embodiment 2-12. Statistical Significance

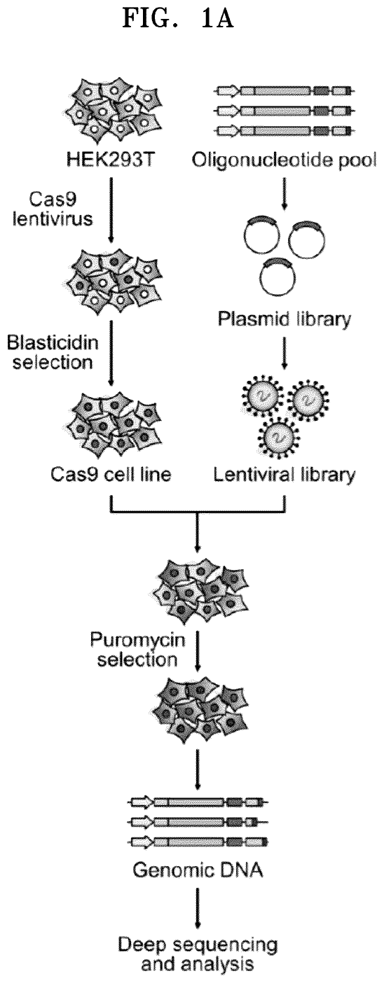

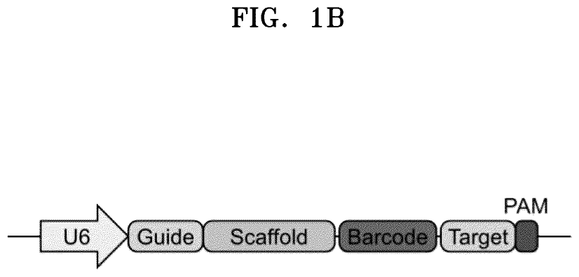

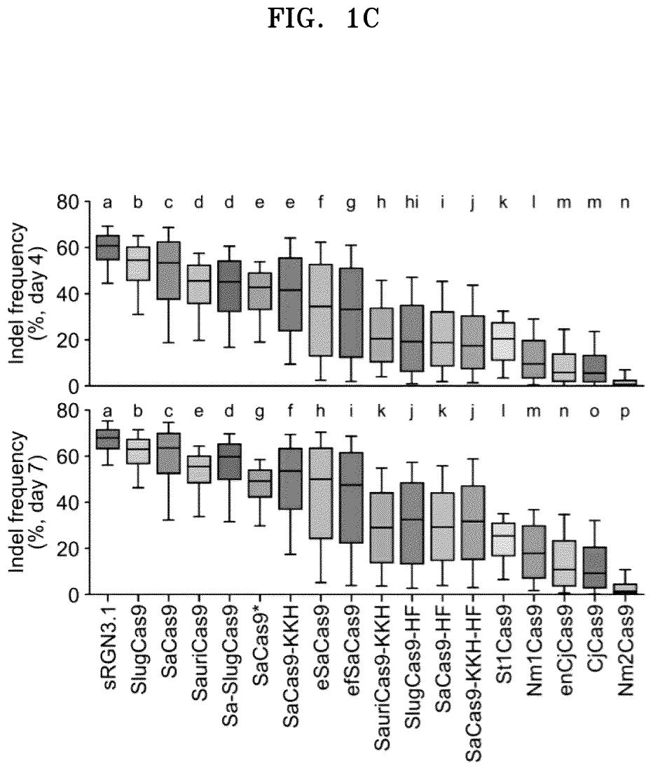

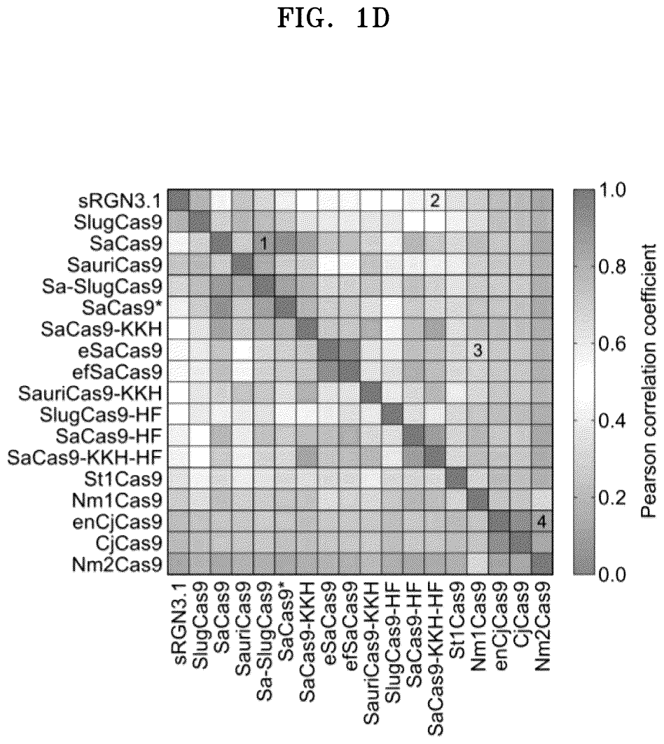

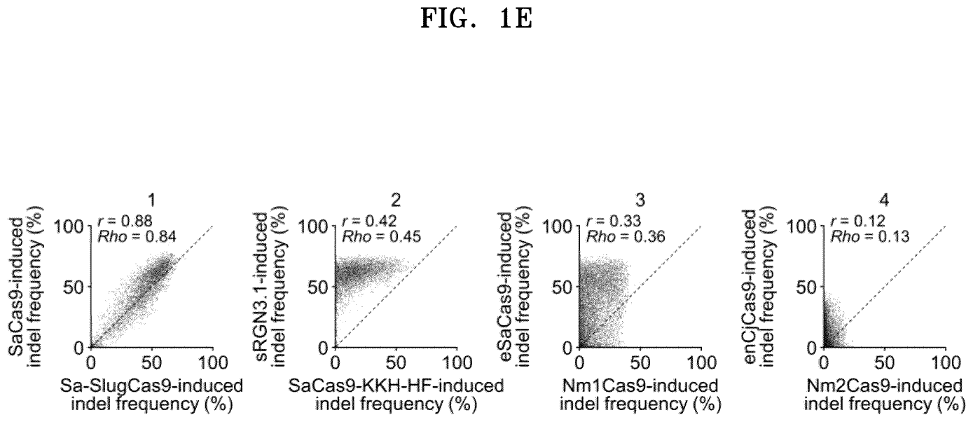

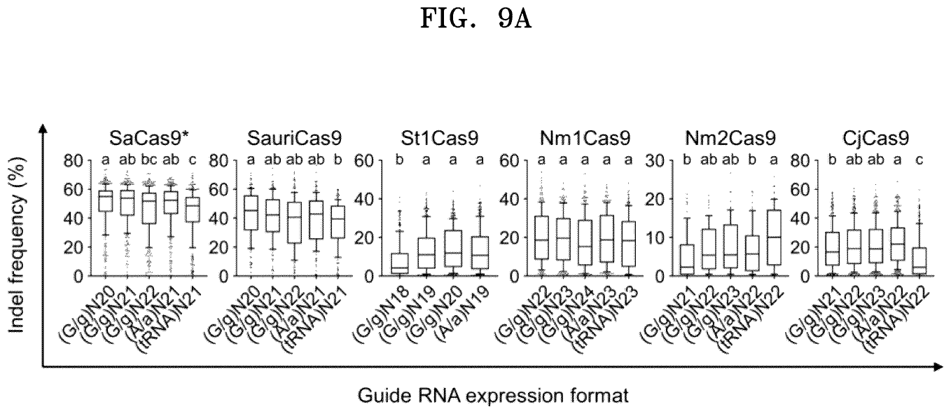

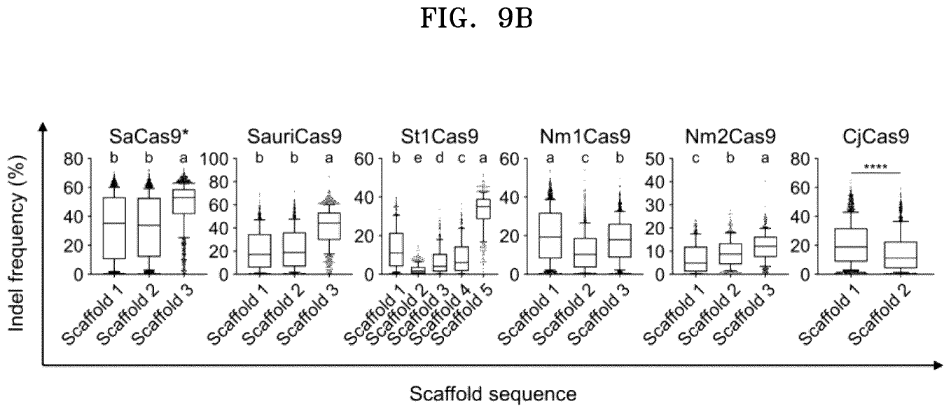

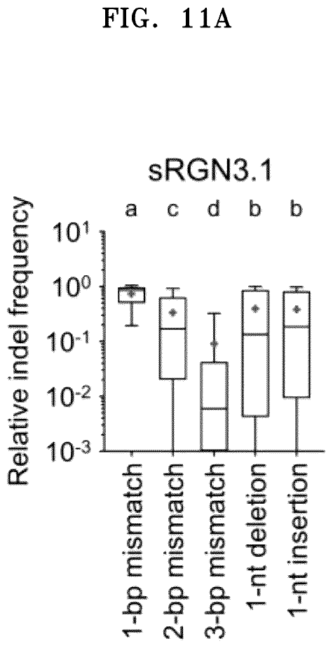

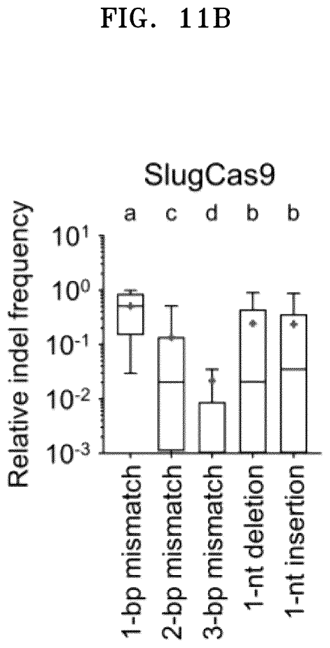

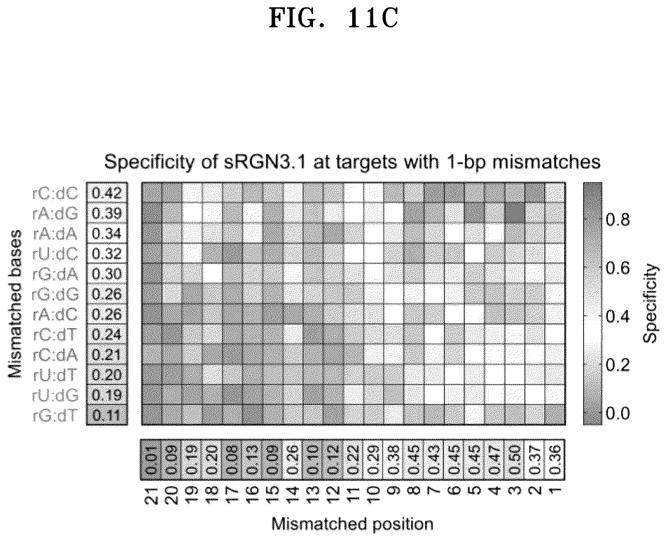

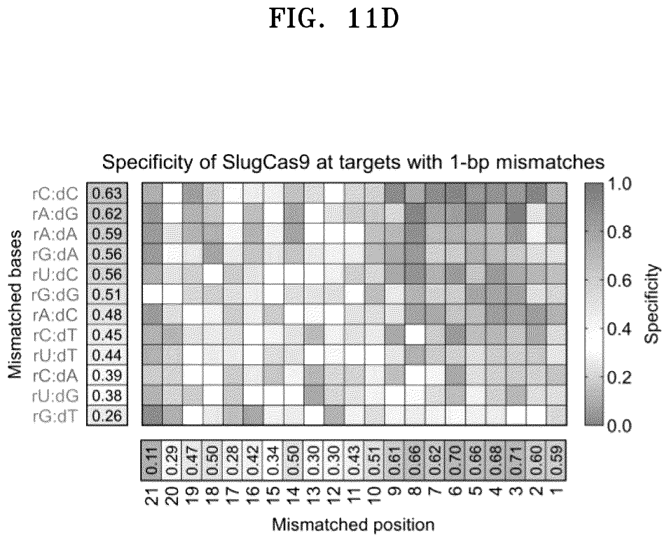

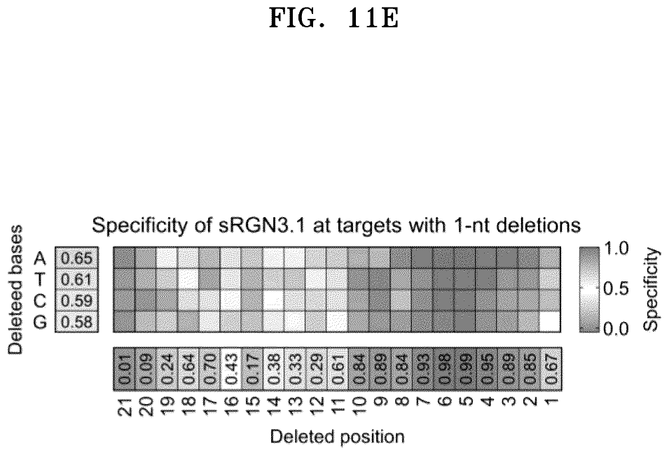

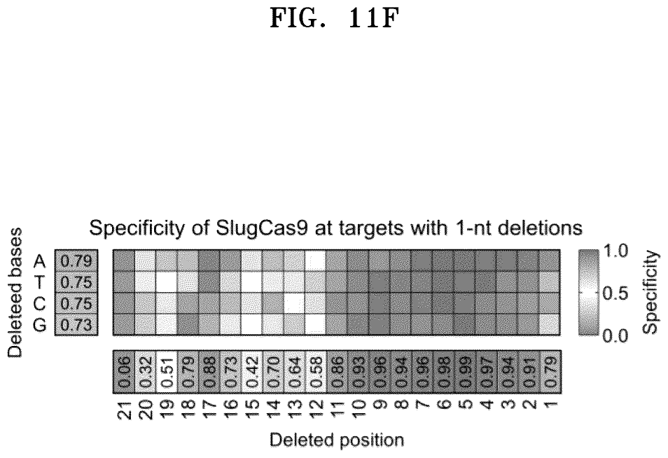

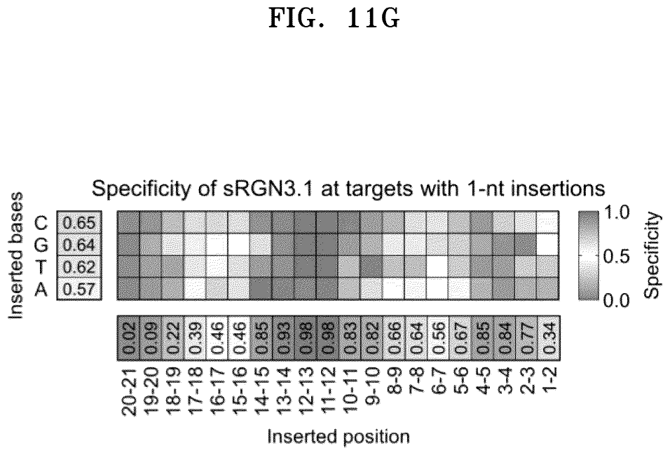

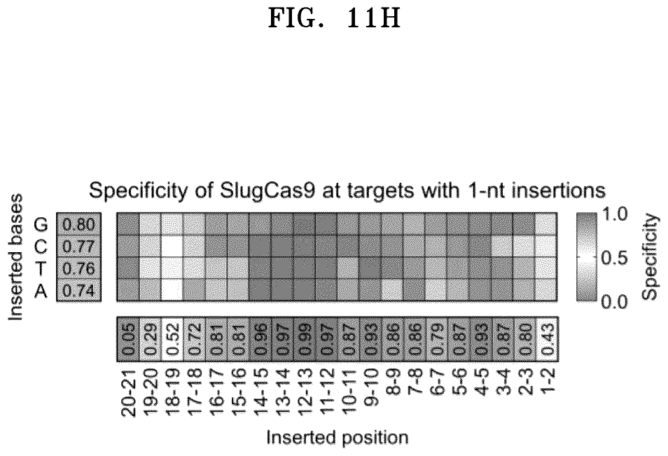

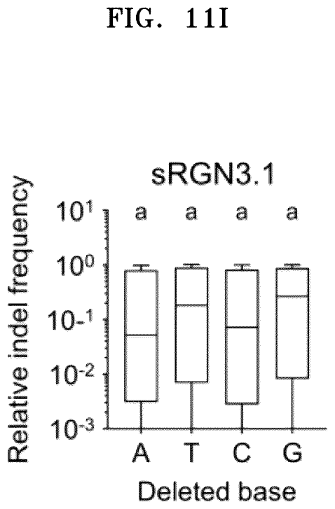

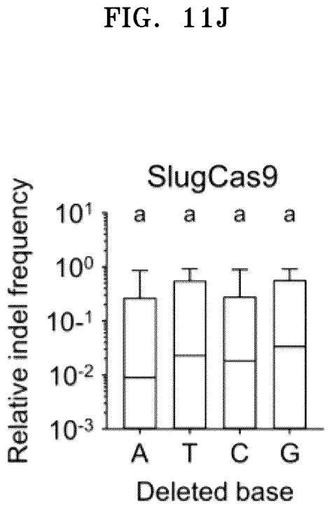

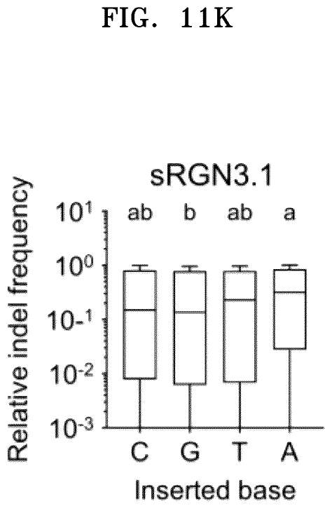

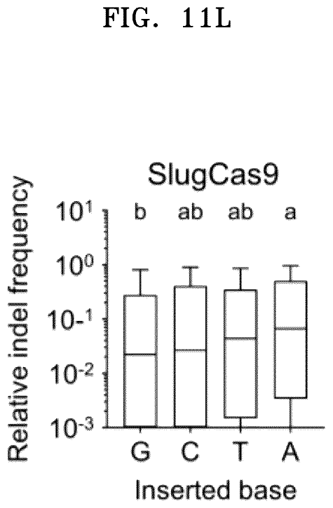

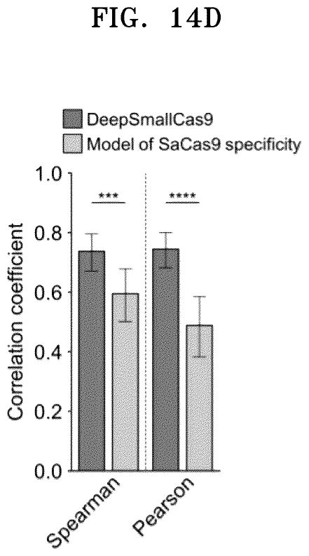

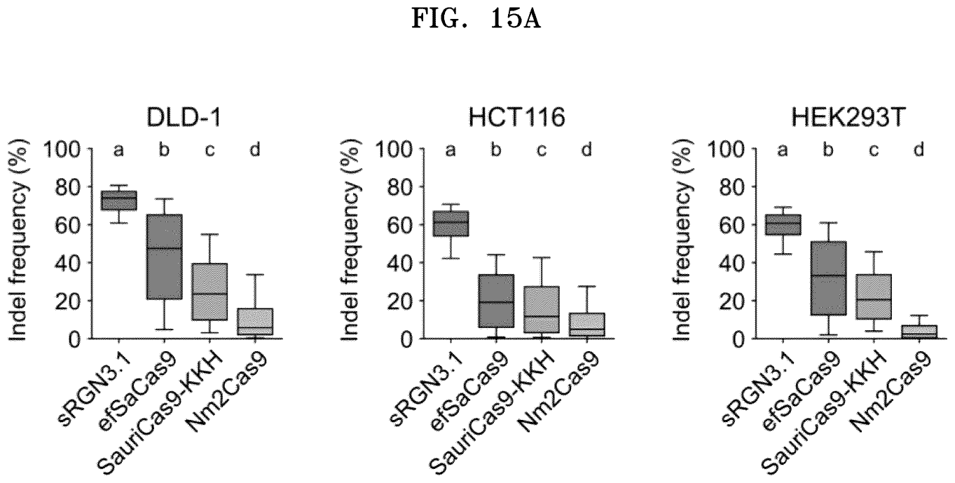

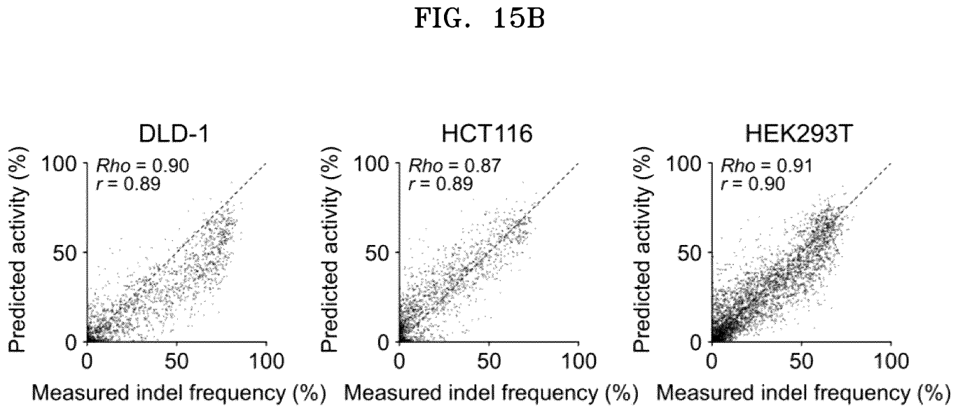

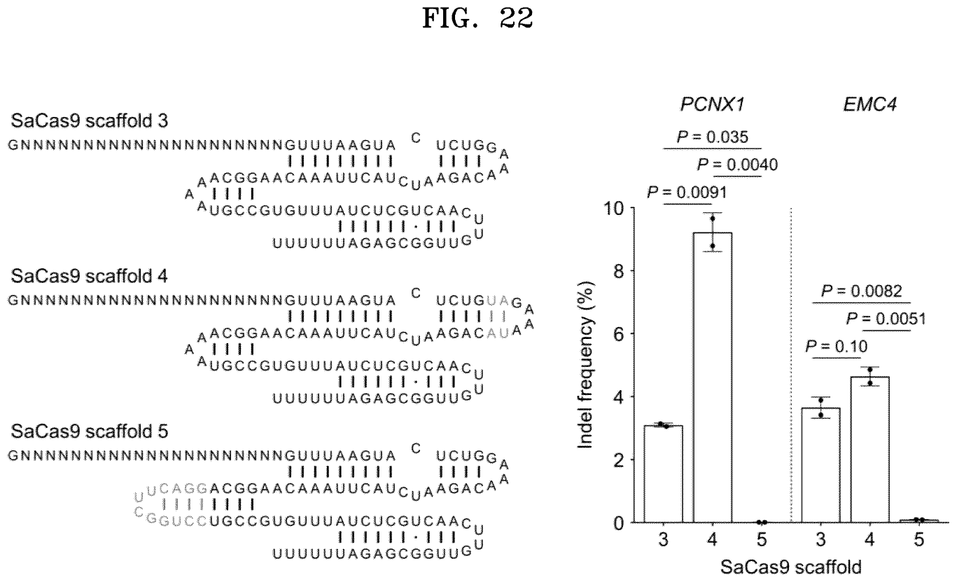

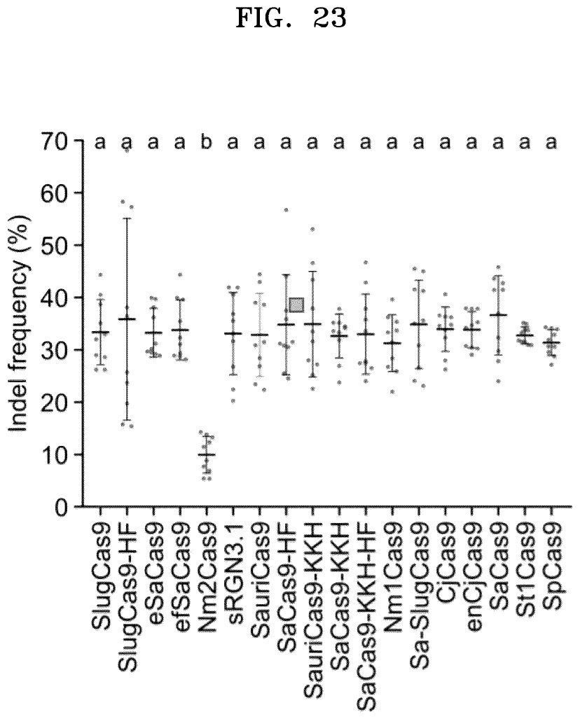

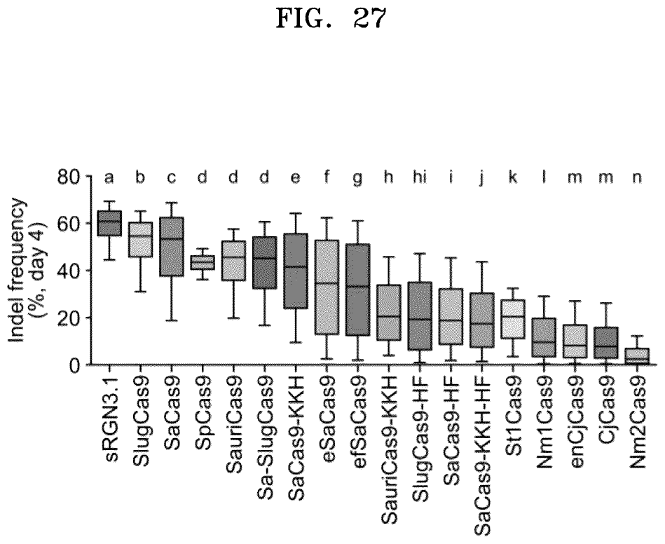

We used one-way analysis of variance or repeated measures one-way analysis variance followed by Bonferroni post-hoc test for multiple comparisons (FIG. 6C, FIG. 1C, FIG. 9, FIG. 22, FIG. 23, FIG. 27, FIG. 11A, 11B, 11I-11L, FIG. 15A). To compare the indel frequencies induced by CjCas9 with CjCas9 scaffolds 1 and 2, we used the two-tailed paired t-test under the null hypothesis that the indel frequencies are the same (FIG. 9B). The two-tailed Steiger's z-test under the null hypothesis that the two correlation coefficients are the same was used to compare the correlation coefficients (FIGS. 14B, 14D). In all the above analyses, a value of P<0.05 was considered to be statistically significant. Statistical significance was calculated using GraphPad Prism 8, IBM SPSS Statistics 27, and cocor.

### Embodiment 2-13. Data Availability

The deep sequencing data used in this study are available at the NCBI Sequence Read Archive under BioProject accession number PRJNA807878.

### Embodiment 2-13. Code Availability

Source codes for DeepSmallCas9 and the custom Python scripts used for the indel frequency calculations are available on Github at <https://github.com/SangyeonSeo/DeepSmallCas9> and <https://github.com/CRISPRJWCHOI/CRISPR_toolkit/tree/master/Indel_searcher_2>, respectively.

### Experimental Example 1. High-Throughput Evaluations of the Activities of 17 the Small Cas9s

To extensively compare the activities of the small Cas9s, we first attempted to generate cell lines that stably express these Cas9s. Because protein expression levels are affected by codon usage, we used codons suggested by GenScript, the recommendations of which previously led to high expression of SpCas9-base editors, unless otherwise specified. The sequences encoding the small Cas9s, with the suggested codons incorporated, were cloned into a lentiviral vector containing the CMV promoter. The resulting lentiviruses were then transduced into HEK293T cells at 0.1 MOI (multiplicity of infection), which should result in only one copy of the small Cas9-encoding sequence per transduced cell (FIG. 1A). Cells that did not express the Cas9s were removed by supplementation of the culture media with blasticidin S at a concentration of 20 μg ml⁻¹, which kills all non-transduced control HEK293T cells, as we previously conducted. When we measured the levels of the small Cas9s using Western blotting, the expression levels were overall comparable to each other except that enCjCas9, Sa-SlugCas9, and Nm1Cas9 showed relatively higher expression levels and that Nm2Cas9, SaCas9-HF, and SlugCas9-HF showed relatively lower expression levels (enCjCas9 Sa-SlugCas9 and Nm sRGN3.1 and St1Cas9 SauriCas9, eSaCas9, and SauriCas9-KKH SaCas9*, CjCas9, efSaCas9, SaCas9, SaCas9-KKH, SaCas9-KKH-HF, and SlugCas9 Nm2Cas9, SaCas9-HF, and SlugCas9-HF) (FIG. 6B, 6C).

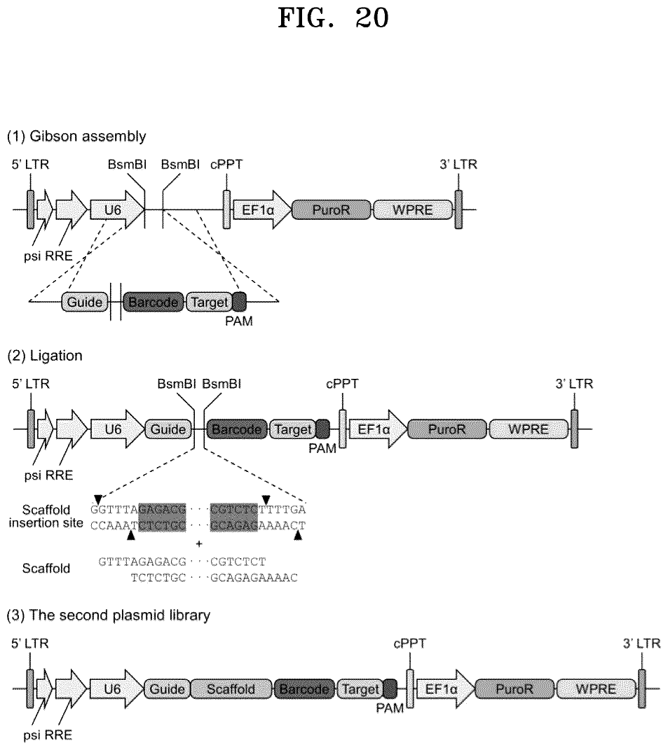

To evaluate the activities of the small Cas9s in a high-throughput manner, we used a pairwise library approach (FIG. 1B), which was previously used to measure the activities of Cas12a, SpCas9 and its variants, base editors, and prime editor 2 at thousands of target sequences. Thirty-one lentiviral libraries named libraries 1-31 were generated from oligonucleotide pools named A, B, C, D, and E, which included 77,712, 55,191, 11,525, 35,990, and 5,402 pairs of guide and target sequences, respectively (FIG. 20). We also employed a previously used lentiviral library named library 32 herein; library 32 was previously named Library A and contains 11,802 pairs of guide and target sequences. Briefly, libraries 1-5, generated using oligonucleotide pool A, were for the evaluation of the activities of the small Cas9s at matched and mismatched target sequences and for the validation of previously reported PAM compatibilities; library 6, prepared using oligonucleotide pool B, was for the evaluation of the activities and PAM compatibilities of SaCas9 and SaCas9-KKH at matched target sequences; libraries 7 to 27, generated using oligonucleotide pool C, were for the determination of optimal spacer lengths and scaffold sequences, as well as for testing various sgRNA transcription formats such as the use of tRNA or a matched or mismatched guanine or adenine at the 5′ terminus (hereafter, for example, 22-nt guide sequences with a matched or mismatched guanine at the 5′ terminus are respectively designated “GN21” or “gN21”); libraries 28-30, prepared using oligonucleotide pool 0, were for the further evaluation of the activities of the small Cas9s at potential off-target sequences and validation of allele-specific gene editing using small Cas9s; library 31, generated using oligonucleotide pool E, was for the evaluation of the activities of SpCas9 at matched target sequences; and library 32 was used to evaluate the activities of SpCas9 at mismatched target sequences and for the validation of PAM compatibilities.

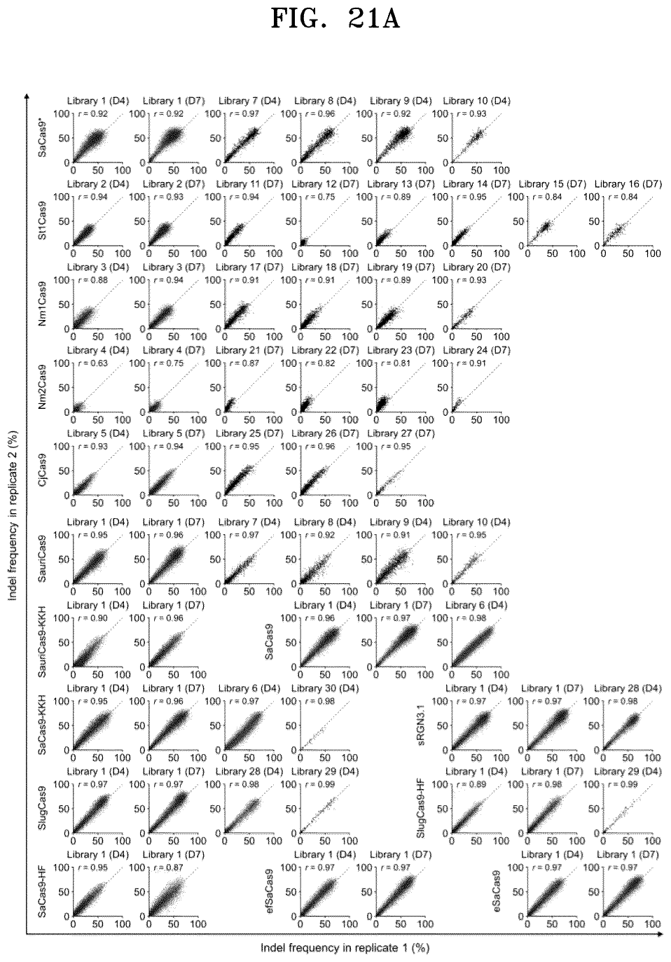

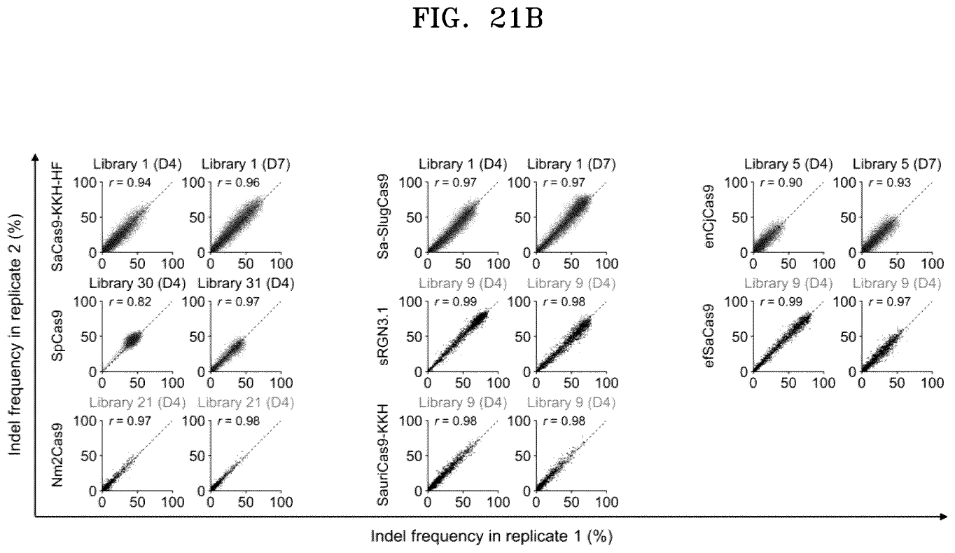

Lentiviral libraries 1-32 were transduced at an MOI of 0.4 into the 19 cell lines expressing the small Cas9s or SpCas9 (FIG. 1A). Four and/or seven days after the transduction, genomic DNA was isolated from harvested cells and subjected to deep sequencing to quantify indel frequencies at the integrated target sequences. Indel frequencies of independently transduced replicates were highly correlated (FIG. 21). Thus, we combined data from two replicates for later analyses to obtain more generalized conclusions.

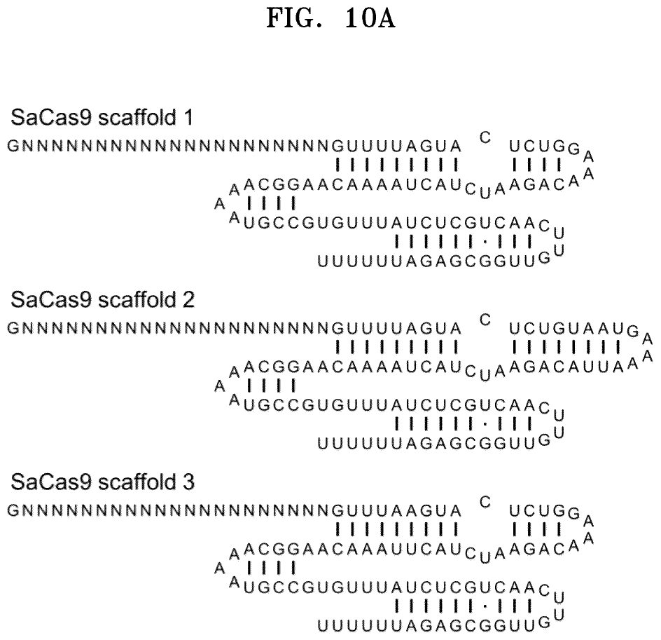

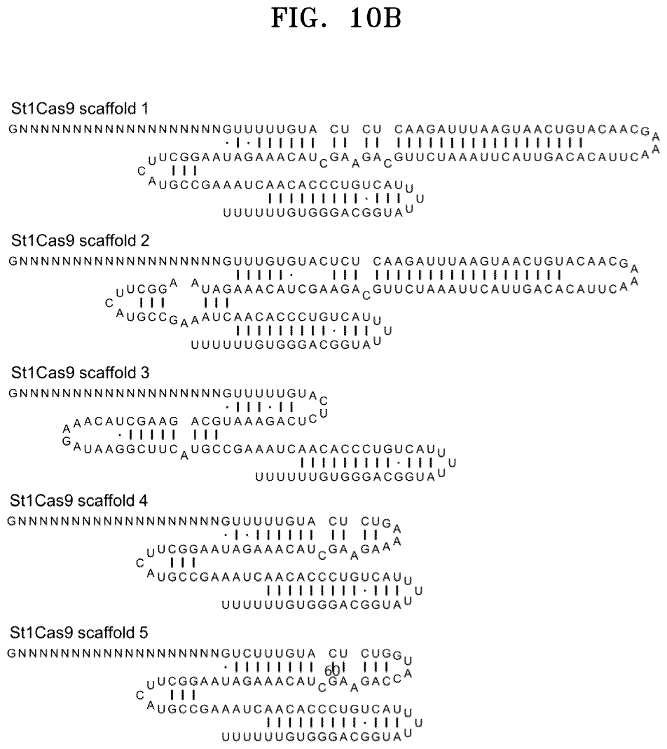

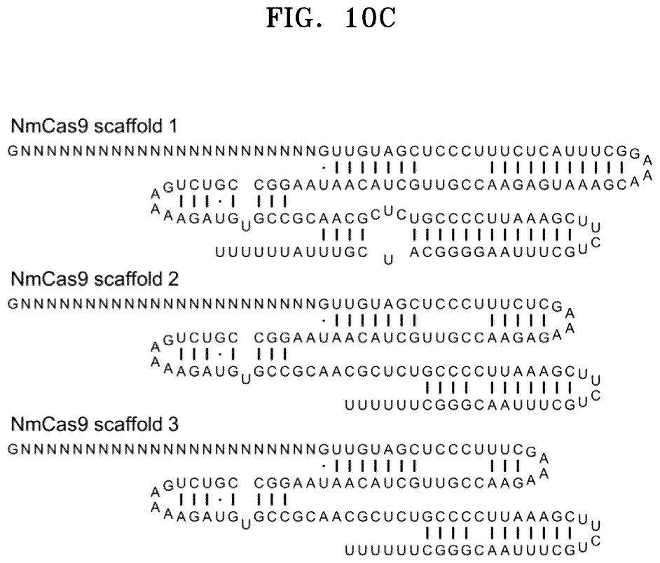

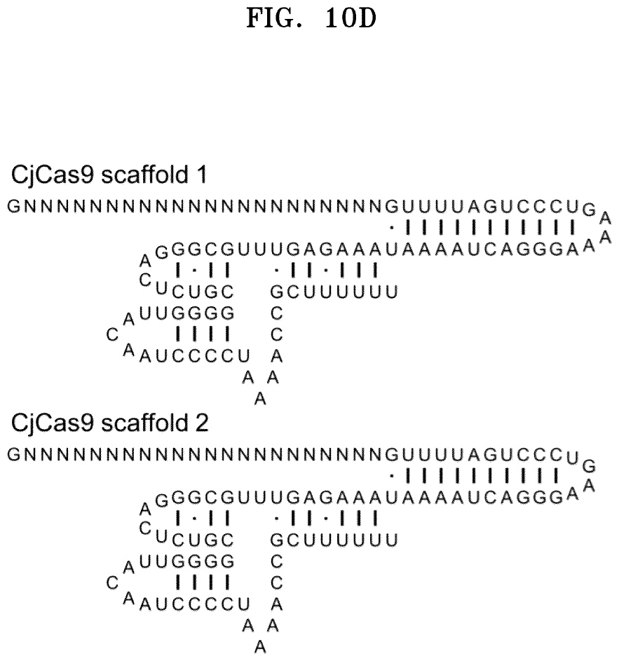

When indel frequencies were measured at target sequences with previously described PAMs, we found that SaCas9, expressed with codons used in the initial study of SaCas9-KKH53, induced higher indel frequencies than did the version expressed with GenScript-recommended codons (hereafter, SaCas9*) (FIG. 10). Thus, we expressed SaCas9, SaCas9-KKH, eSaCas9, efSaCas9, SaCas9-HF, and SaCas9-KKH-HF using the codons used in the initial study of SaCas9-KKH for the subsequent evaluations unless otherwise noted. The general activities of the small Cas9s at sites with previously characterized PAM sequences at day 4 were ranked as follows: sRGN3.1>SlugCas9>SaCas9>SauriCas9 and Sa-SlugCas9>SaCas9* and SaCas9-KKH>eSaCas9>efSaCas9>SauriCas9-KKH≥SlugCas9-HF≥SaCas9-HF>SaCas9-KKH-HF>St1Cas9>Nm1Cas9>enCjCas9 and CjCas9>Nm2Cas9 (FIG. 1C).

During high-throughput screening involving lentiviral vectors, barcodes and guide sequences in the vectors can be shuffled at a frequency that depends on the length of a common sequence located between the two elements; this phenomenon probably occurs because the lentiviral reverse-transcriptase exhibits a template switching activity. In our constructs, no common sequence was located between the barcodes and target sequences, but an 83- to 143-bp length of common sequence containing the scaffold was present between the guide sequences and the barcode-target sequences. We analyzed genomic DNA from cells transduced with library 6 to determine the switching rate in this situation. We found that the guide sequences and barcode-target sequences became uncoupled at a rate of about 3%, similar to lentiviral switching rates previously reported given the short length of sequence (92 bp) between the two elements. The shuffled targets would essentially never undergo small Cas9-induced cleavage because the expressed sgRNAs and targets would almost never match. Therefore, we would observe an indel frequency that would be 97% (=100%−3%) of the actual indel frequency (i.e., if the actual indel frequency were 30%, we would observe an indel frequency of 30%×97%=29%).

### Experimental example 2. Correlations between the Activities of the Small Cas9s

Next, we ascertained if the relative activities of these small Cas9s were affected by the sequence compositions of the protospacers. A comparison of the small Cas9-induced indel frequencies at each protospacer sequence revealed that such differences between the indel frequencies frequently depended on the protospacer sequence compositions; for example, at some protospacer sequences, Nm1Cas9 and eSaCas9 induced similar indel frequencies, whereas at others, Nm1Cas9 induced much lower indel frequencies, resulting in a poor correlation between SaCas9- and Nm1Cas9-induced indel frequencies (FIG. 1D, 1E). When we summarized the correlations between the activities of the small Cas9s, the Pearson correlation coefficients between the activities of Staphylococcus-derived small Cas9s (sRGN3.1, SlugCas9, SaCas9, SauriCas9, Sa-SlugCas9, SaCas9*, SaCas9-KKH, eSaCas9, efSaCas9, SauriCas9-KKH, SlugCas9-HF, SaCas9-HF, and SaCas9-KKH-HF) and those between the activities of Campylobacter jejuni-derived Cas9s (CjCas9 and enCjCas9) were relatively high (range, 0.42 to 0.93), whereas all of the other cases showed poor correlations (range, 0.12 to 0.42) although Neisseria meningitidis-derived Cas9s (Nm1Cas9 and Nm2Cas9) are from different strains of the same species (Neisseria meningitidis) (FIG. 1D, 1E). Together, these findings show that at specific target sequences, the relative activities of the small Cas9s will not necessarily agree with the general activity ranks described above.

These poor correlations imply that the target sequence compositions associated with high nuclease activities for a given small Cas9 may differ from those of the other small Cas9s. To find the sequence features associated with the activity of each small Cas9, we employed XGBoost combined with SHAP using the features that had been used for Cas9 activity predictions in the past, such as all position-independent and position-dependent mononucleotides and dinucleotides, as well as additional features. The 20 most critical features for activity predictions for each of the small Cas9s are shown in FIG, 7. Notable findings include the following. First, the most important features were associated with the PAM sequences for all small Cas9s with the exception of SaCas9-HF and SaCas9-KKH-HF, for which the minimum free energy (MFE) of the sgRNA was the most important feature and characteristics of the PAM sequences were the third and second most important features, respectively. Second, the number of TT dinucleotides was a disfavored feature for all small Cas9s, presumably because an abundance of T repeats in the guide sequence could decrease the efficiency of RNA polymerase III-dependent transcription, potentially due to premature termination of sgRNA transcription. The same finding was also previously observed for SpCas9 and its variants and prime editor 2. Third, another important feature, common to all small Cas9s, was the MFE of the sgRNA, although this feature was the 96th, 23rd, 21st, and 96th most important feature for Nm1Cas9, enCjCas9, CjCas9, and Nm2Cas9, respectively. This result is in line with the finding that a high MFE of the sgRNA is associated with high SpCas9 activity. Fourth, except for features associated with the PAM, the number of TTs, and the MFE of the sgRNA, position-dependent mononucleotides and, less frequently, dinucleotides constitute the majority of important features for all small Cas9s and only a limited fraction of these features were shared between the small Cas9s. Fifth, members of the groups of Staphylococcus-derived Cas9s, Campylobacter jejuni-derived Cas9s, and Neisseria meningitidis-derived Cas9s frequently shared important features within each group, whereas the important features for St1Cas9, Nm1Cas9, and Nm2Cas9 were also frequently unique for each Cas9. For example, out of the 12 Staphylococcus-derived Cas9s, 10 Cas9s (excluding SauriCas9 and SlugCas9-HF) shared 10-A (A at position 10, favored), 10 Cas9s (excluding eSaCas9 and efSaCas9) shared 2-C (favored), eight Cas9s (excluding sRGN3.1, SlugCas9, SaCas9-KKH, and eSaCas9) shared 6-G (disfavored), and eight Cas9s (excluding SaCas9, SaCas9-KKH, SauriCas9-KKH, and SlugCas9-HF) shared GC count (extremely high or low GC counts were disfavored) as important features. In addition, enCjCas9 and CjCas9 shared 3-C (favored), number of Ts (disfavored), and Tm of positions 1-8 (favored) as important features. Nm1Cas9 and Nm2Cas9 shared 8-G (favored), 10-T (disfavored), 10-G (favored), and number of CGs (disfavored) as important features although the correlation between the activities of these small Cas9s was relatively poor (the Pearson correlation coefficient=0.31), which could be partly attributable to the low activities of Nm2Cas9. Among the top 20 features for each small Cas9, the number of unique important features for each Cas9 were as follows; two for sRGN3.1, two for SlugCas9, zero for SaCas9, one for SauriCas9, one for Sa-SlugCas9, zero for SaCas9-KKH, one for eSaCas9, two for efSaCas9, one for SauriCas9-KKH, five for SlugCas9-HF, two for SaCas9-HF, zero for SaCas9-KKH-HF, seven for St1Cas9, eight for Nm1Cas9, zero for enCjCas9, one for CjCas9, and 11 for Nm2Cas9. Taken together, these results are compatible with the finding that the correlations between the activities of the small Cas9s are relatively low, except for Staphylococcus-derived Cas9s and Campylobacter jenuni-derived Cas9s.

### Experimental Example 3. Determination of PAM Compatibilities for the Small Cas9s

Previously, the PAM compatibilities of each small Cas9 were separately determined using cleavage assays either in vitro or in bacterial cells, although the PAM compatibilities in bacterial and mammalian cells can sometimes be slightly different. Furthermore, these separate evaluations in different experimental settings cannot be used to decide which small Cas9 should be used for target sequences with a given PAM sequence, especially in human cells. Thus, we compared the PAM compatibilities of the small Cas9s together in human cells in one experimental setting.

Using the high-throughput analysis, we tested candidate PAM sequences that were at least one nucleotide (nt) longer than the previously characterized PAM sequences. For example, in the case of SaCas9, known to recognize NNGRRT as the PAM sequence, we attempted to evaluate NNNNNNN sequences as PAM candidates. However, this approach would require us to test 4⁷=16,384 candidates, which are too many to be practical, so we tested 80 7-nt PAM sequences (64 NNNNNTN+16 NNGRRNN; the nucleotides that were evaluated in the PAM are underlined hi bold). Thus, for Staphylococcus-derived Cas9s, we examined indel frequencies at 2,400 (=80×30) target sequences, which are a combination of 80 7-nt PAM sequences (64 NNNNNTN+16 298 NNGRRNN) and 30 protospacer sequences previously tested for SaCas9. In the same manner, 80 candidate PAMs (64 NNNNNAN+16 NNRGANN) for St1Cas9, 80 candidate PAMs (64 NNNNNNNTN+16 NNNNGATNN) for Nm1Cas9, 64 candidate PAMs (NNNNNNN) for Nm2Cas9, and 80 candidate PAMs (64 NNNNNNNCN+16 NNNNRYANN) for CjCas9 and enCjCas9 were combined with previously tested 303 protospacers for each small Cas9 and evaluated.

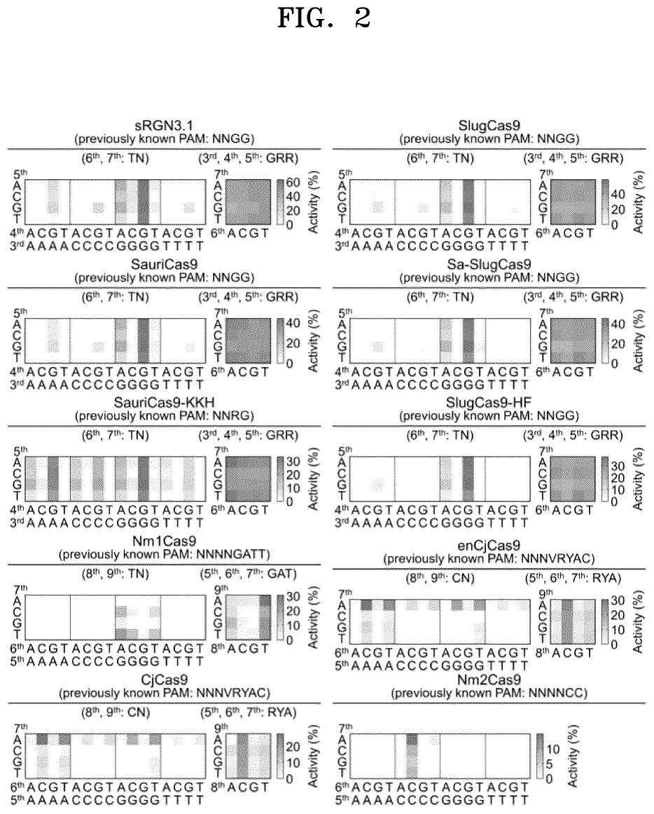

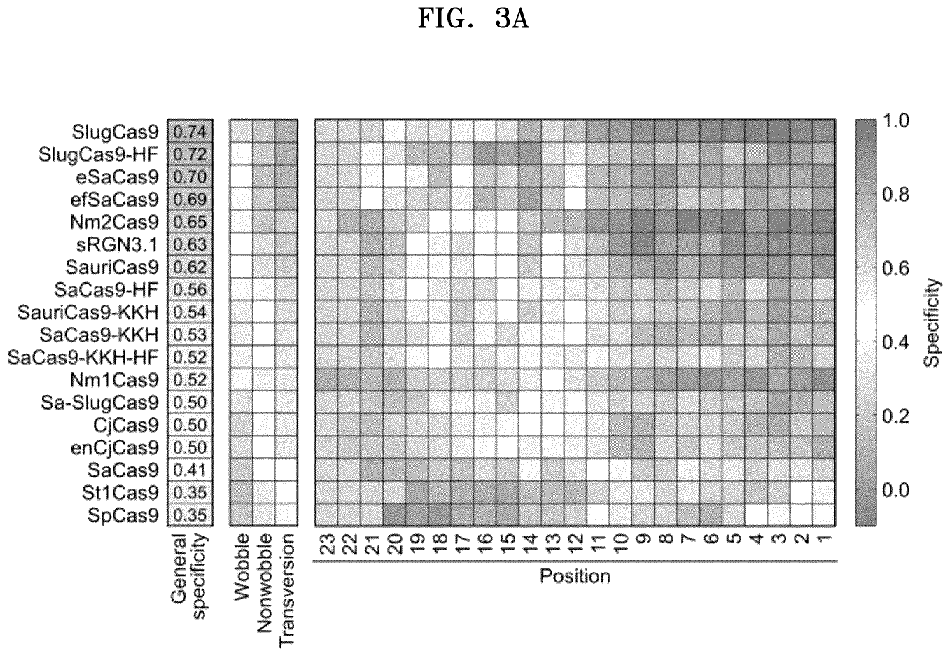

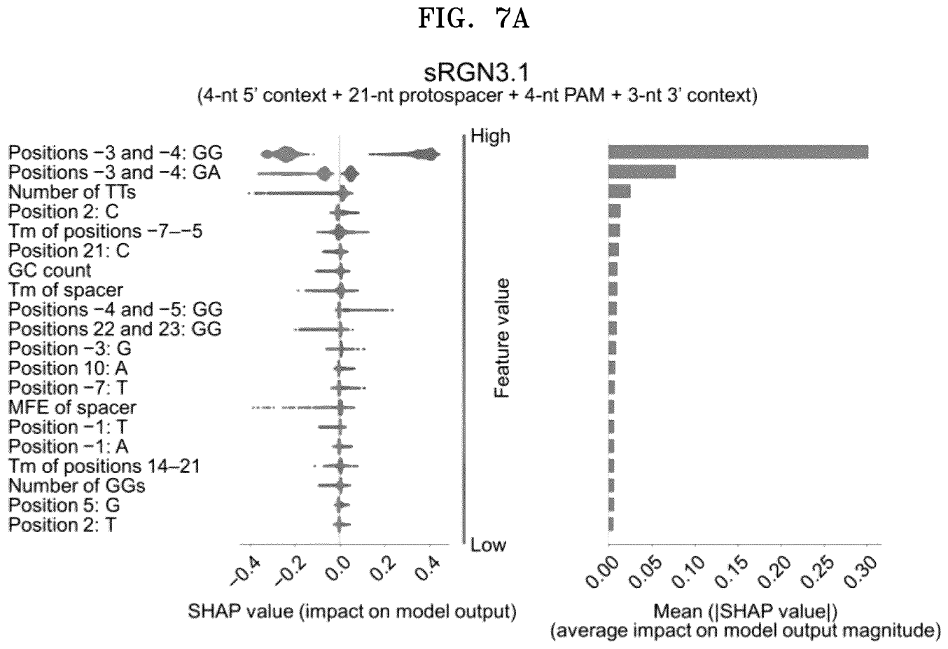

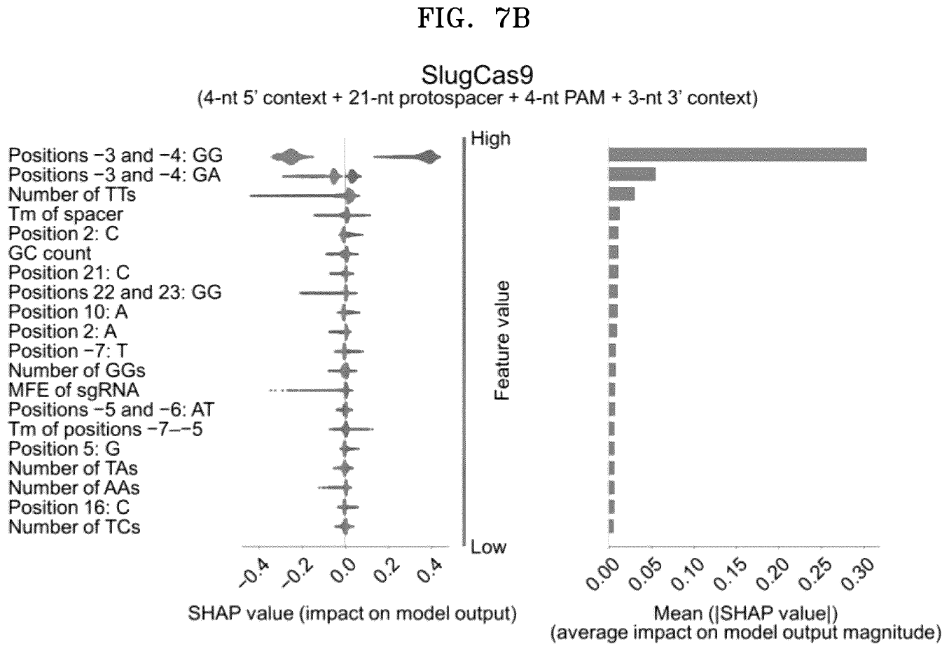

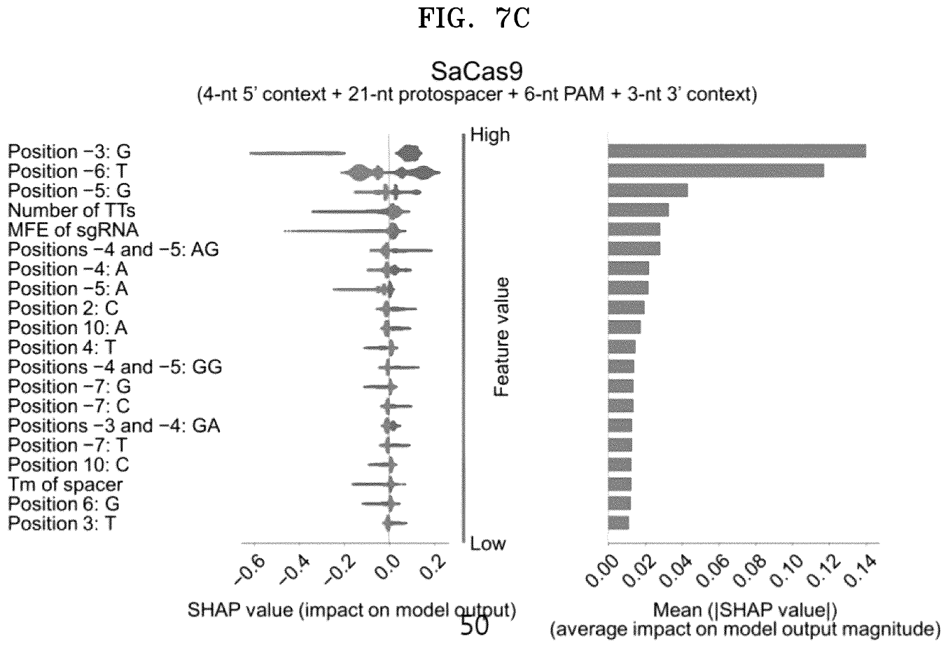

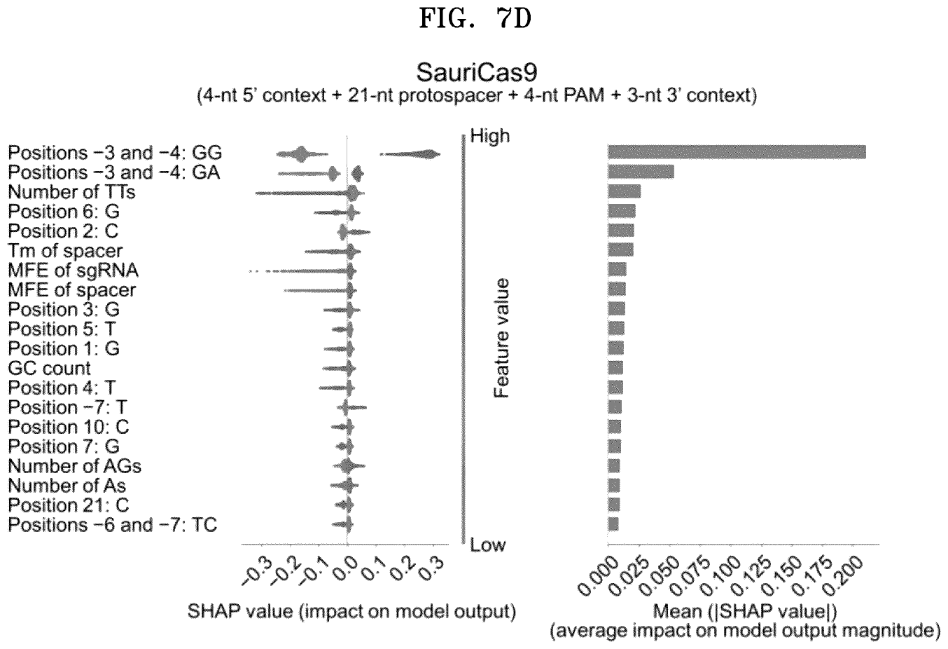

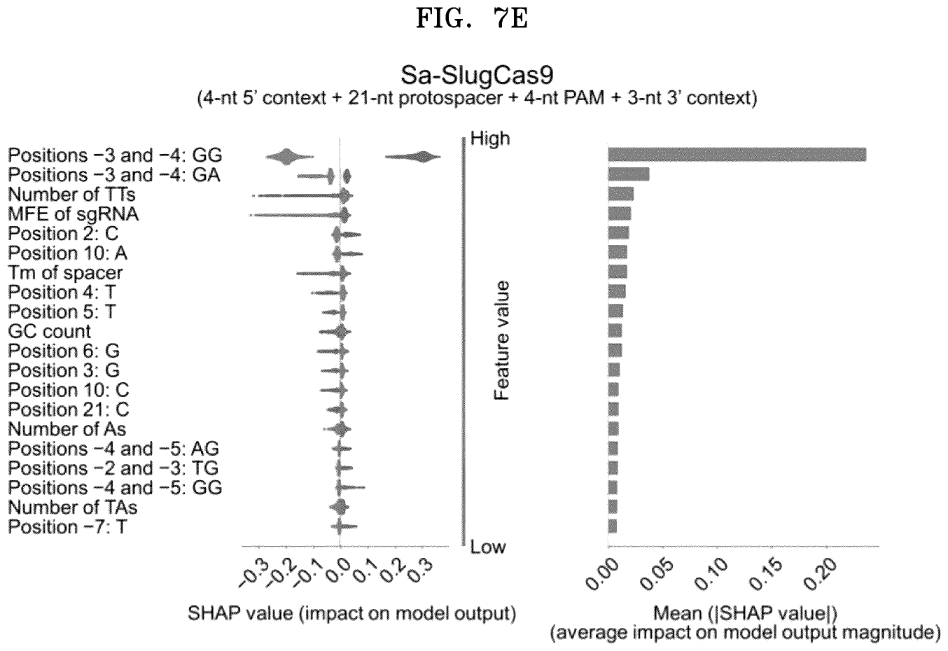

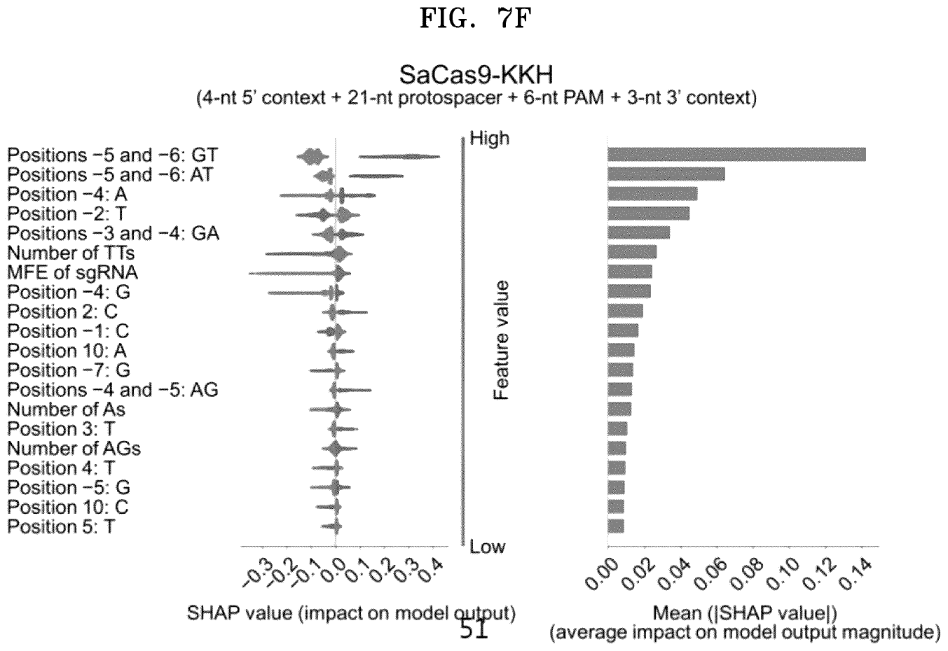

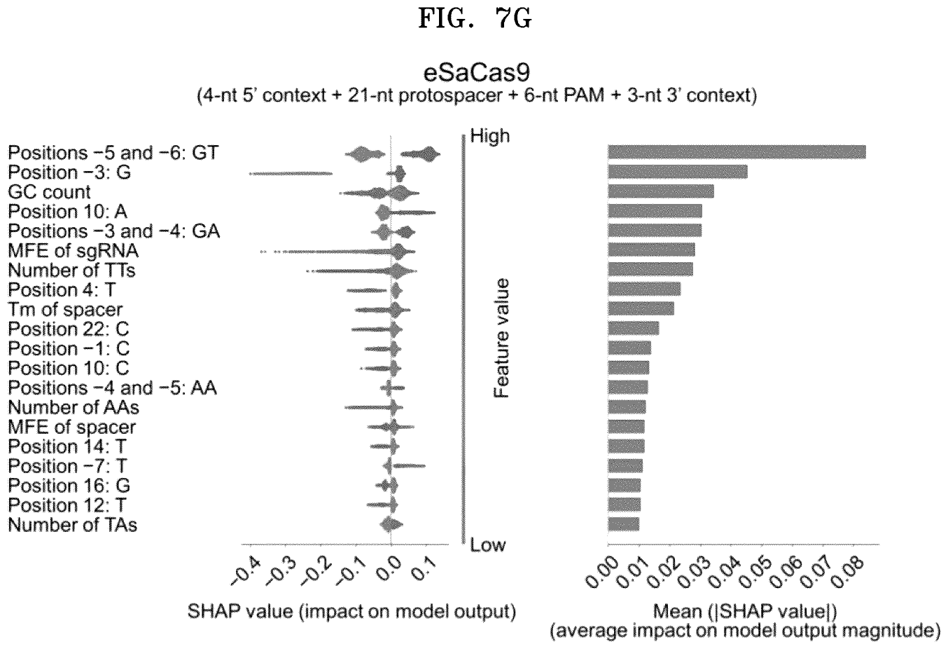

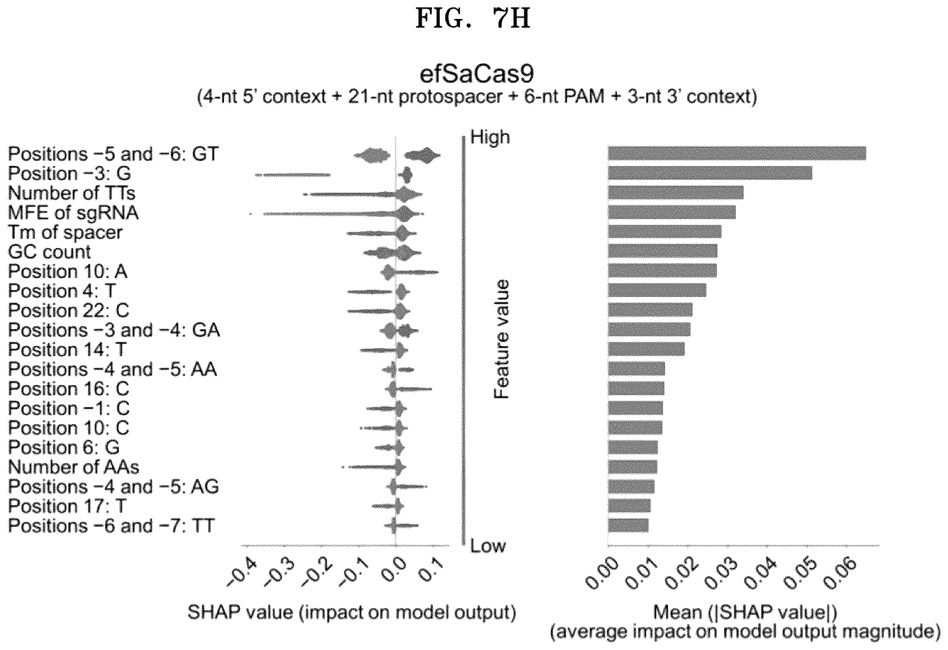

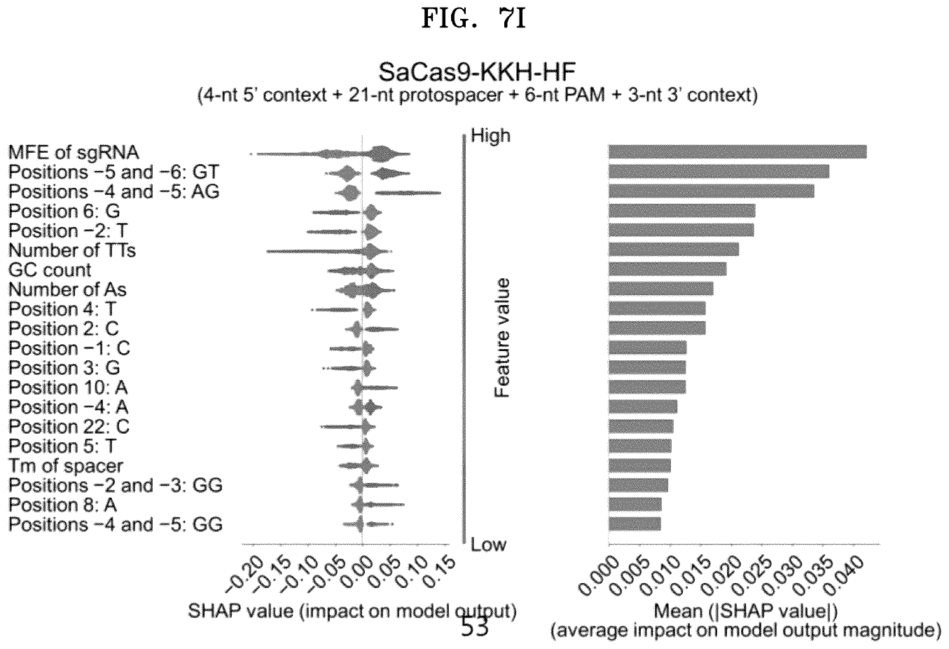

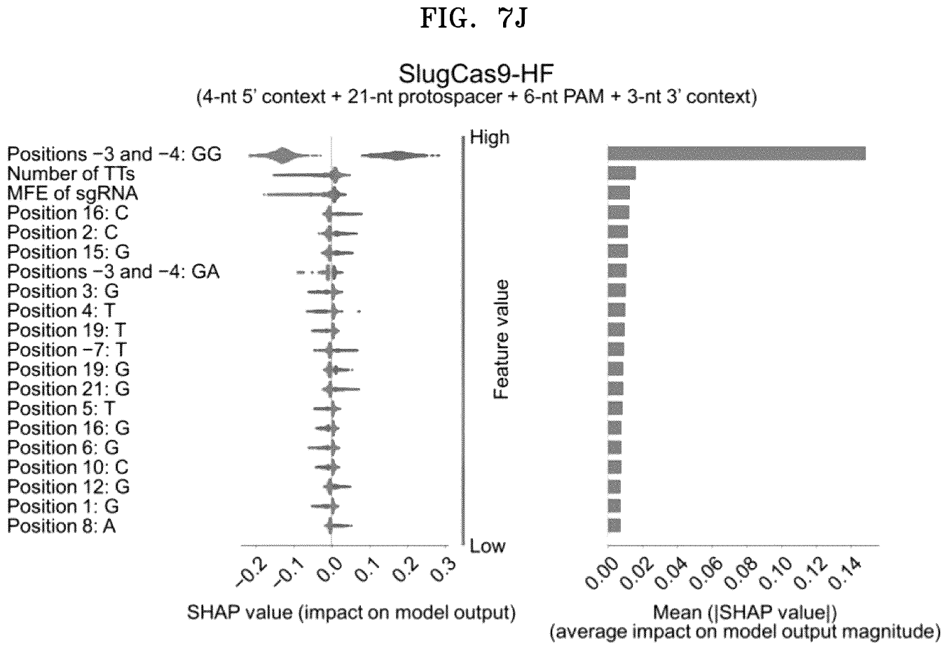

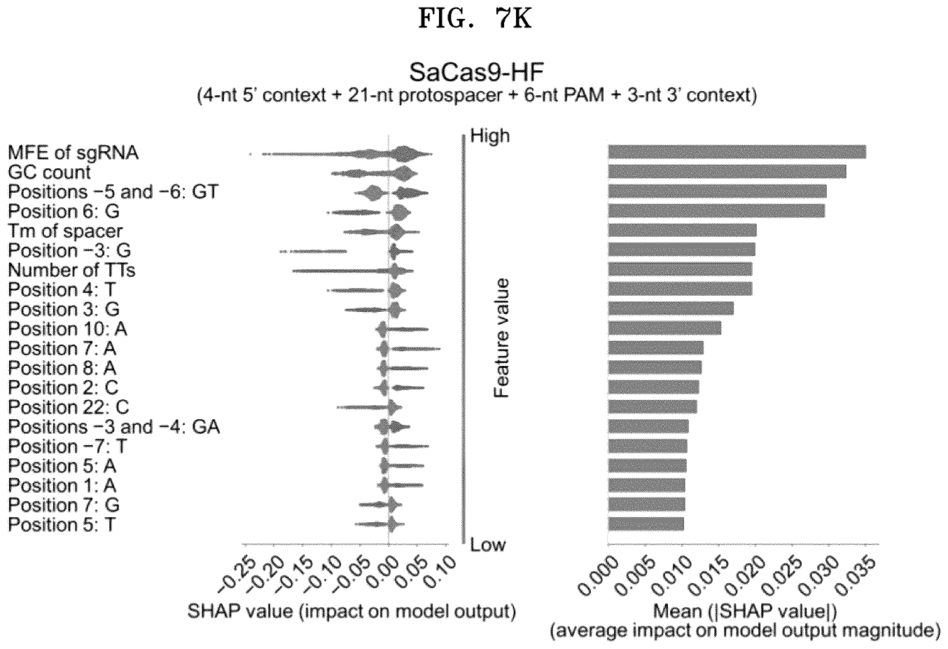

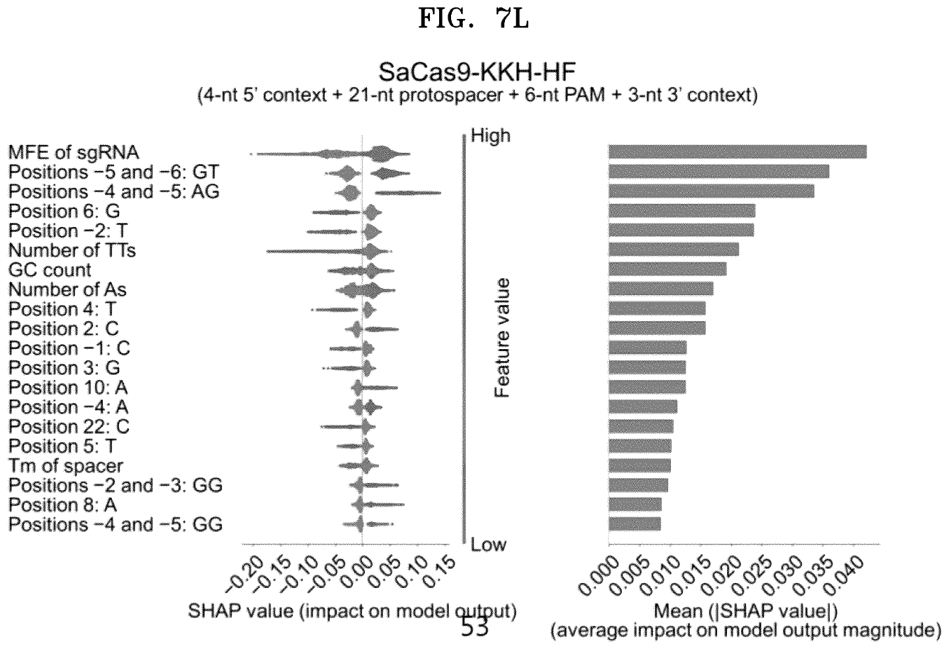

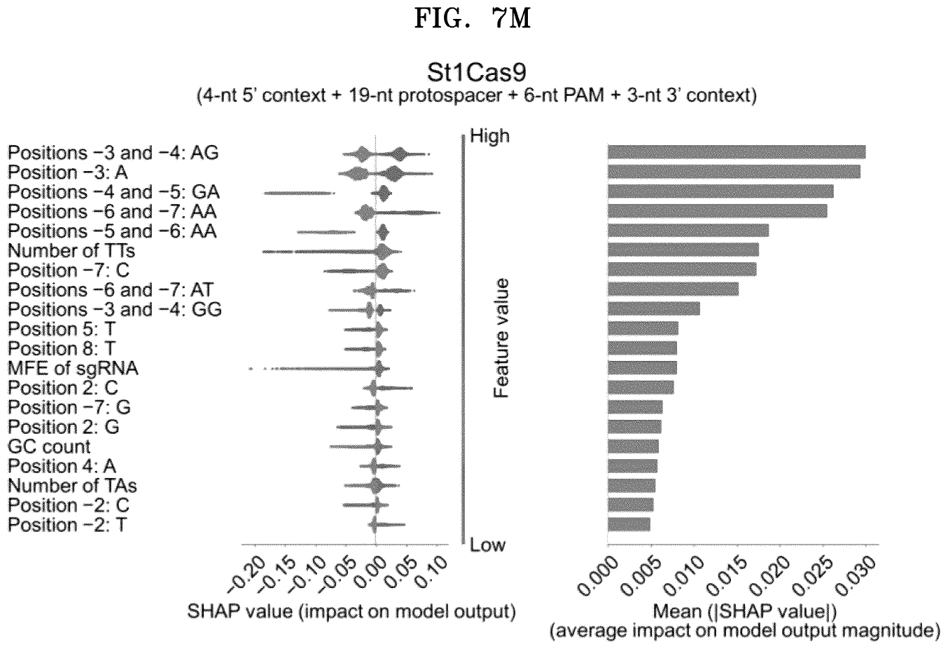

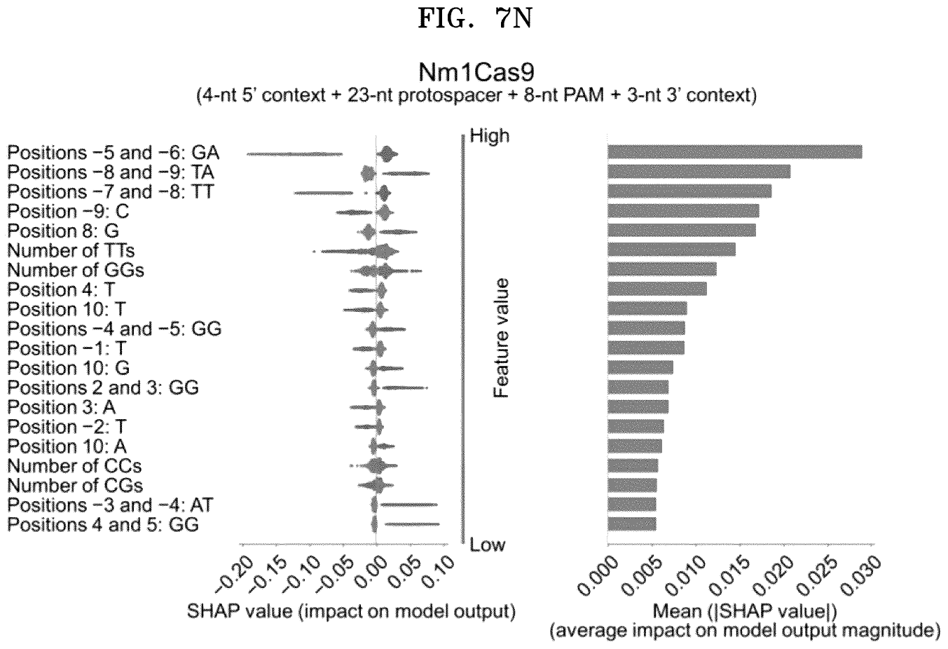

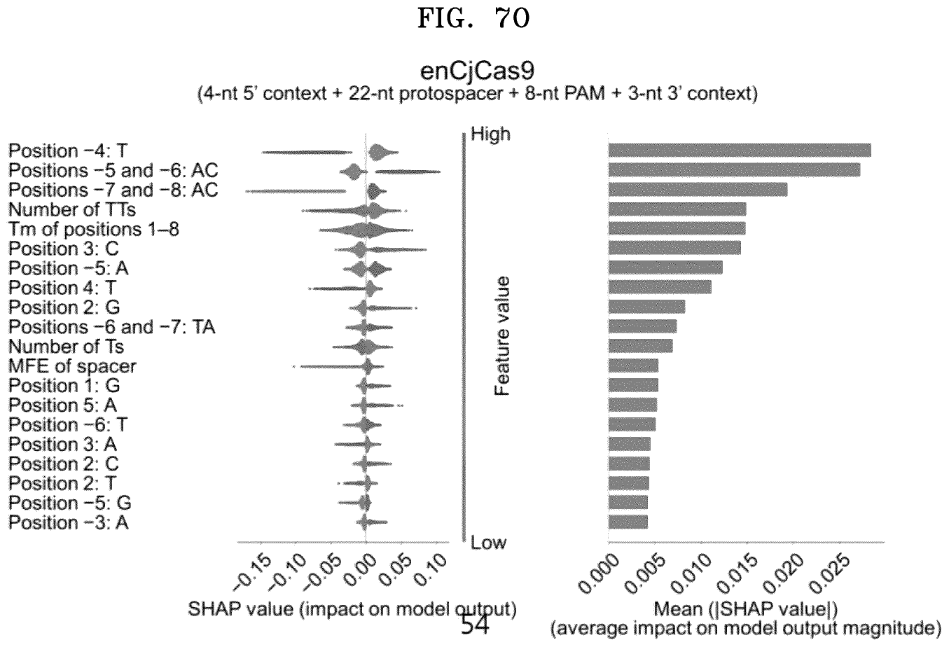

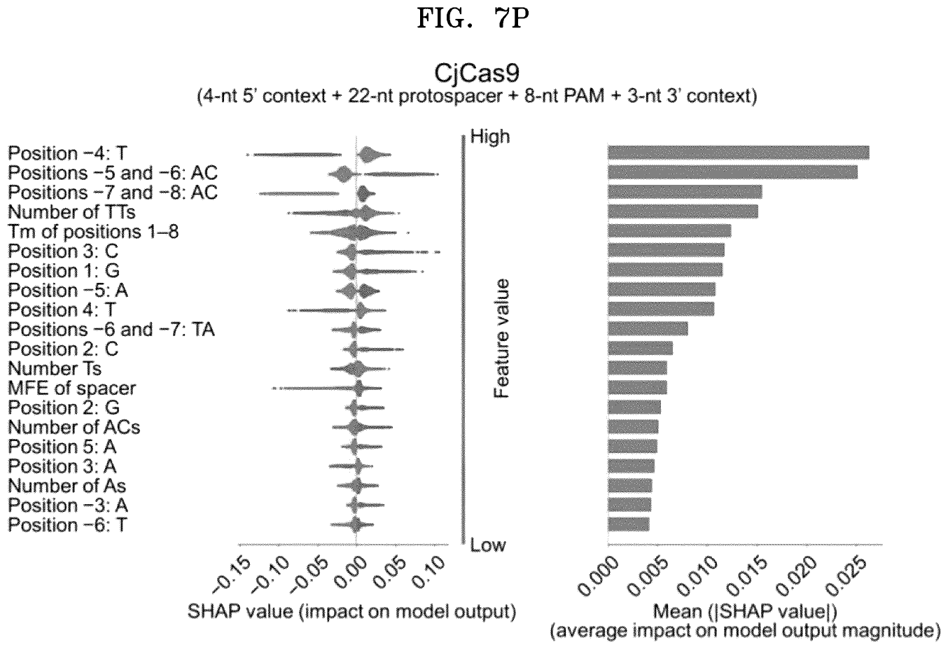

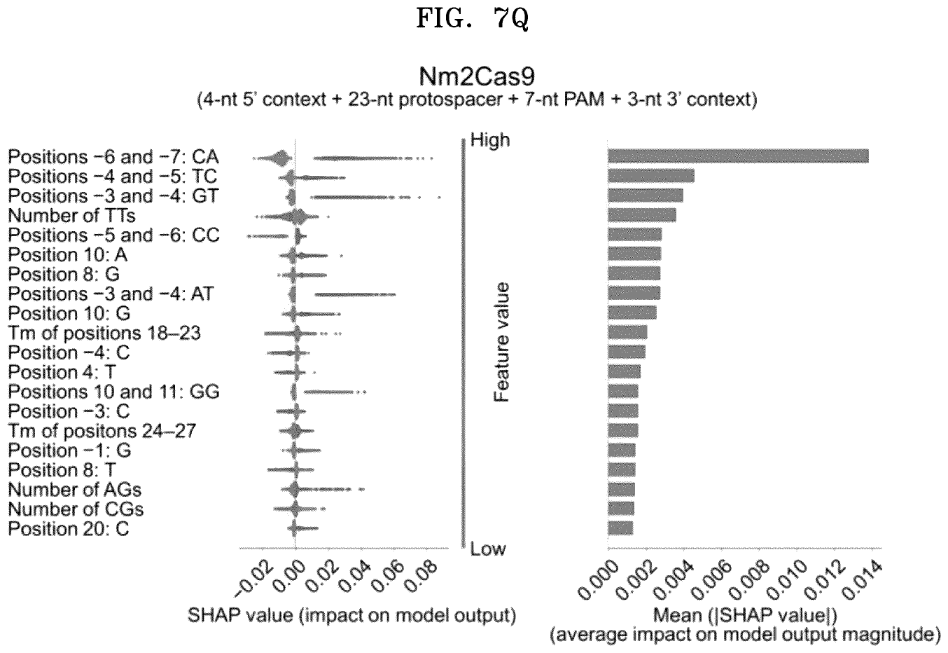

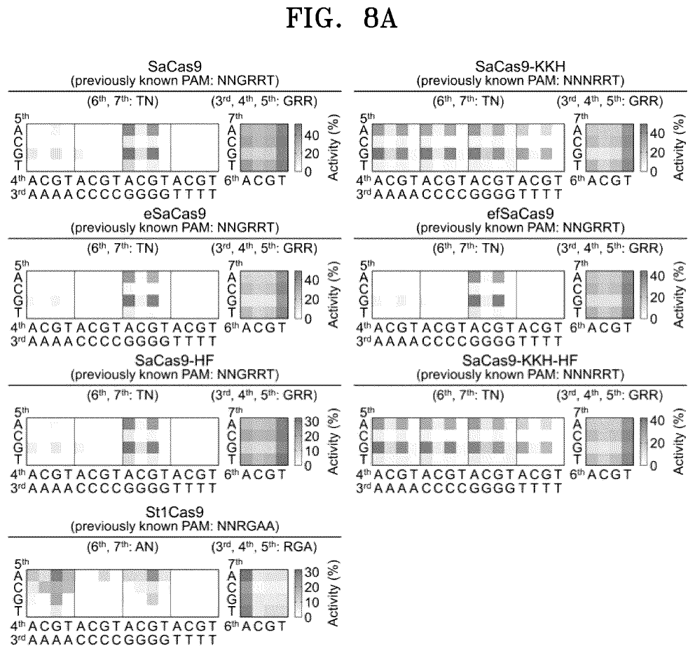

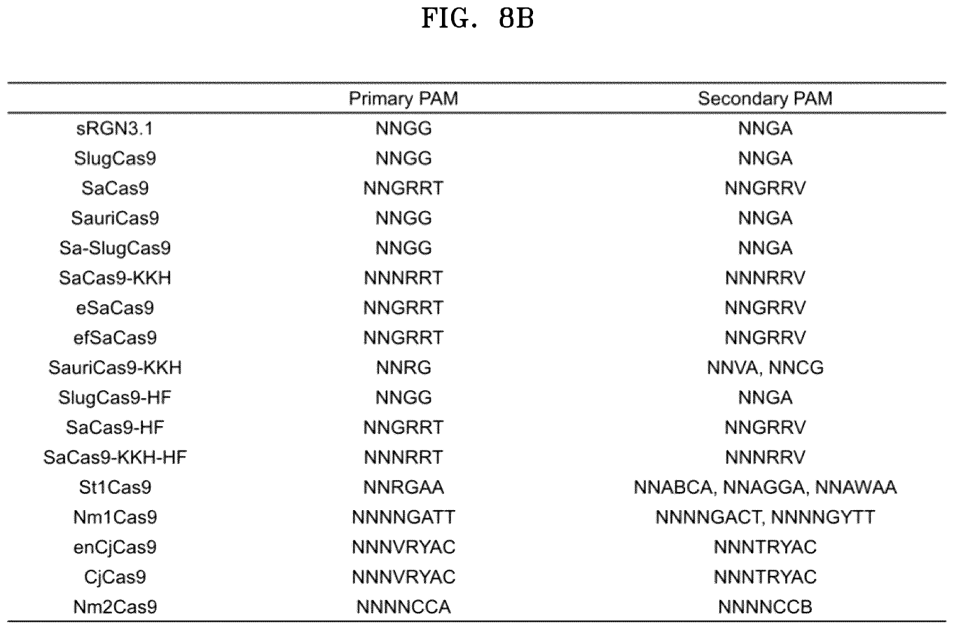

Based on the observed indel frequencies, we determined the PAM compatibilities and classified PAM sequences as primary or secondary (FIG. 2 and FIG. 8). Although the PAM compatibilities we observed were generally in line with previously reported results, there were some notable differences. For example, sRGN3.1, SlugCas9, SauriCas9, Sa-SlugCas9, and SlugCas9-HF were found to recognize NNGA as a secondary PAM and SauriCas9-KKH was found to recognize NNVA and NNCG as secondary PAMs. Nm1Cas9 recognizes NNNNGACT and NNNNGYTT as secondary PAMs, whereas Nm2Cas9 recognizes NNNNCCA and NNNNCCB as primary and secondary PAMs, respectively. In addition, we found that both CjCas9 and enCjCas9 disfavor T at the fourth position of the PAM (FIG. 7), rendering NNNVRYAC and NNNTRYAC primary and secondary PAMs, respectively, of these two small Cas9s, which is in line with the results of recent in vitro cleavage assays.

### Experimental example 4. Optimization of sgRNA Expression Formats and Scaffold Sequences

As an attempt to maximize the activities of the small Cas9s, we then compared several sgRNA expression formats for these small Cas9 orthologues. In previous studies of these small Cas9s, a U6 promoter was generally used to drive sgRNA expression and sgRNAs included 18- to 23-nt guide sequences with a guanine at the 5′ terminus that either matched or did not match the target sequence. As an alternative format for the sgRNAs, we could shorten or lengthen the guide sequence, use an adenine (A/a) instead of a guanine (G/g) to initiate U6 promoter-driven transcription, or utilize tRNA-mediated cleavage to generate a perfectly matched sgRNA regardless of the first nucleotide of the target sequence. To find the most efficient sgRNA expression format for genome editing, we tested four to five different formats for each small Cas9 at thousands of target sequences. When we determined the average editing efficiencies for each sgRNA expression format, we found that (G/g)N20 is the most efficient sgRNA expression format for SaCas9*, SauriCas9, and St1Cas9, although the differences between this format and the second most efficient sgRNA expression formats ((G/g)N21 for SaCas9* and SauriCas9 and (G/g)N19 for St1Cas9) were not statistically significant (FIG. 9A). For Nm1 Cas9 and Nm2Cas9, (G/g)N22 and (tRNA)N22 sgRNAs showed the highest efficiencies, respectively, although (G/g)N22 previously showed the highest activities for Nm2Cas9 when it was tested at only two target sequences. In the case of CjCas9, (A/a)N22 sgRNAs showed the highest efficiencies. For SaCas9, SauriCas9, and CjCas9, tRNA-mediated generation of perfectly matched sgRNAs resulted in the lowest efficiencies, which is compatible with the finding that (tRNA)N20 sgRNAs were associated with lower SpCas9 efficiencies than were (G/g)N19 sgRNAs

We also tested several (two to five) different scaffold sequences for each small Cas9 (FIG. 10). For SaCas9, three different scaffolds have previously been used, but it remains unknown which one is associated with the highest efficiencies. Our high-throughput comparison showed that SaCas9 scaffold 3, which lacks the UUUU sequence in the middle of the scaffold sequence, is associated with the highest SaCas9* efficiencies (FIG. 9B). In the case of SauriCas9, SaCas9 scaffold 1 was previously used51. However, our results revealed that SaCas9 scaffold 3 is linked with the highest efficiencies. For St1Cas9, we tested four different scaffolds (SO Cas9 scaffolds 1-4) that showed relatively high efficiencies in a previous study and another scaffold that was used in a recent study (St1Cas9 scaffold 5); out of the five scaffold sequences, St1Cas9 scaffold 5, which is relatively short and lacks the UUUU sequence in the middle, showed the highest efficiencies. For Nm1Cas9, NmCas9 scaffolds 1-3 have previously shown relatively good efficiencies and our high-throughput analyses showed that NmCas9 scaffold 1 is associated with the highest activities. For Nm2Cas9, NmCas9 scaffold 1 has previously been used; however, our high-throughput results showed that NmCas9 scaffold 3, the shortest among the three NmCas9 scaffolds, was associated with the highest Nm2Cas9 activities. In the case of CjCas9, CjCas9 scaffolds 1 and 2 have been used in previous studies but it was unknown which one would be associated with higher activity. Our high-throughput study has now shown that CjCas9 scaffold 1 is associated with higher activities.

Furthermore, to improve the activities of the Staphylococcus-derived Cas9s, we engineered the SaCas9 scaffold by extending the repeat:anti-repeat duplex (to create SaCas9 scaffold 4) or by extending the first hairpin with a superstable loop (to create SaCas9 scaffold 5) (FIG. 22), changes previously applied to the SpCas9 scaffold. When we tested SaCas9 scaffolds 3-5 with two guide sequences targeting two endogenous sites (PCNX1 and EMC4), sgRNAs with scaffold 4 showed the highest activities at both targets although the difference was not statistically significant at the EMC4 target, whereas those with SaCas9 scaffold 5 showed almost no activity at either target. We propose SaCas9 scaffold 4 as a promising alternative scaffold for Staphylococcus-derived Cas9s.

### Experimental Example 5. High-Throughput Profiling of the Activities of the Small Cas9s and SpCas9 at Mismatched Target Sequences

To compare the fidelities of the small Cas9s and SpCas9, we determined the relative frequencies of indels induced by the small Cas9s and SpCas9 at mismatched target sequences normalized to the frequencies at matched targets. Indel frequencies were determined four days after lentiviral libraries 1-5 designed in the current study or library 32 used in our previous study had been transduced into Cas9-expressing HEK293T cells. Within the libraries 1-5, we included 2,340-2,820 sgRNA-target pairs per small Cas9 (30 sgRNAs+78-94 targets with various mismatches or no mismatch). The 30 sgRNAs were chosen based on the results of previous studies to avoid extremely inefficient sgRNAs. The pairs were designed to allow the evaluation of the effects of several variables (the number, position, and type of mismatched nucleotides) on the activities of the small Cas9s and SpCas9; every possible 1-bp mismatch at each protospacer position was included. However, different Cas9s induced different frequencies of indels at matched target sequences. These drastic differences between the activities at matched target sequences could bias the comparison of activities at mismatched target sequences. Therefore, out of the 30 sgRNAs, for each small Cas9 and SpCas9, we selected ten that were associated with similar indel frequencies (with average values that ranged from 31% to 37%) at matched target sequences, as we did previously, except in the case of Nm2Cas9 (FIG. 23). Nm2Cas9 induced very low indel frequencies at all 30 protospacers (with the ten selected sgRNAs, the average of the Nm2Cas9-induced indel frequencies was 10%).

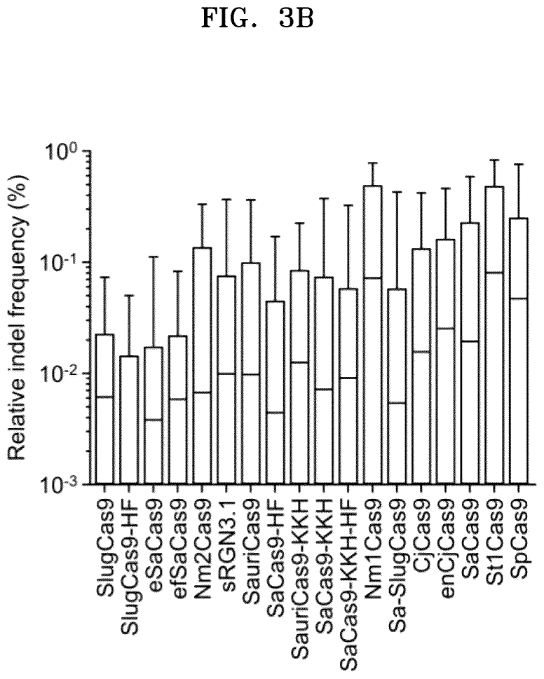

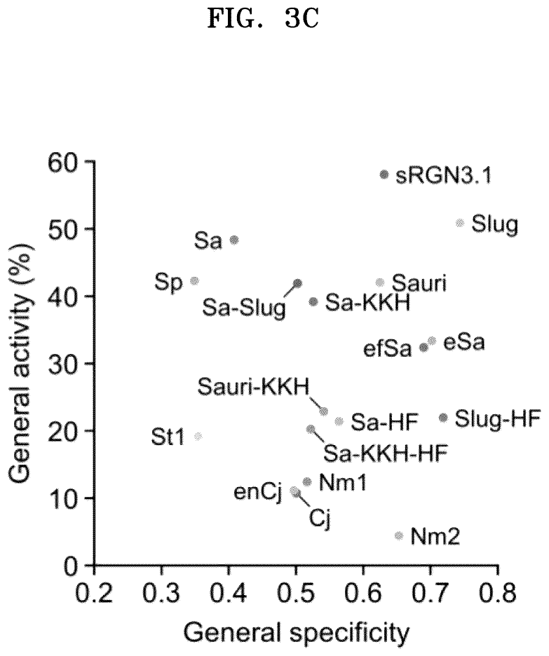

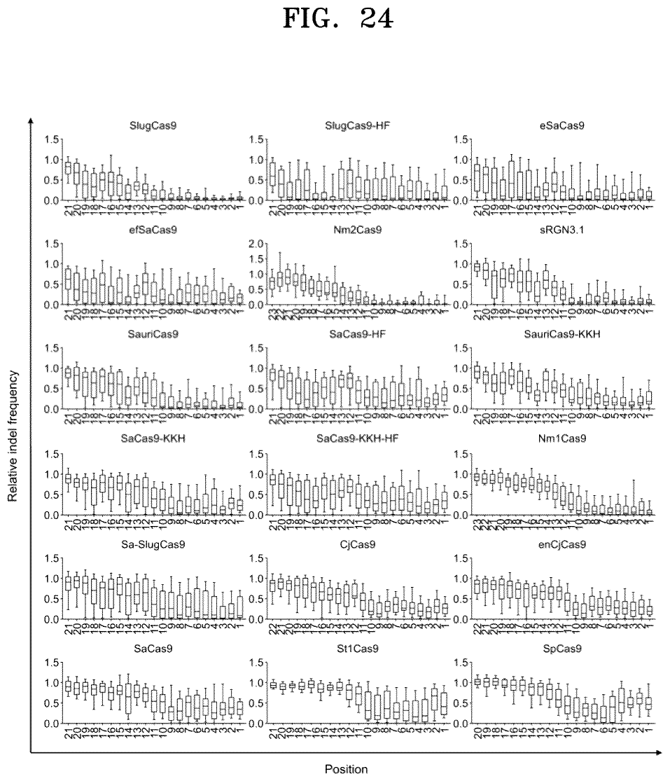

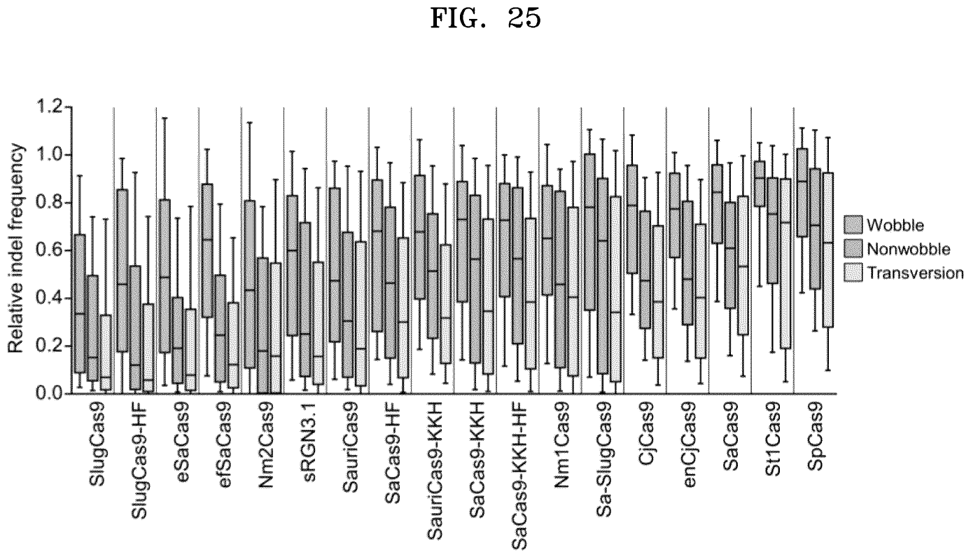

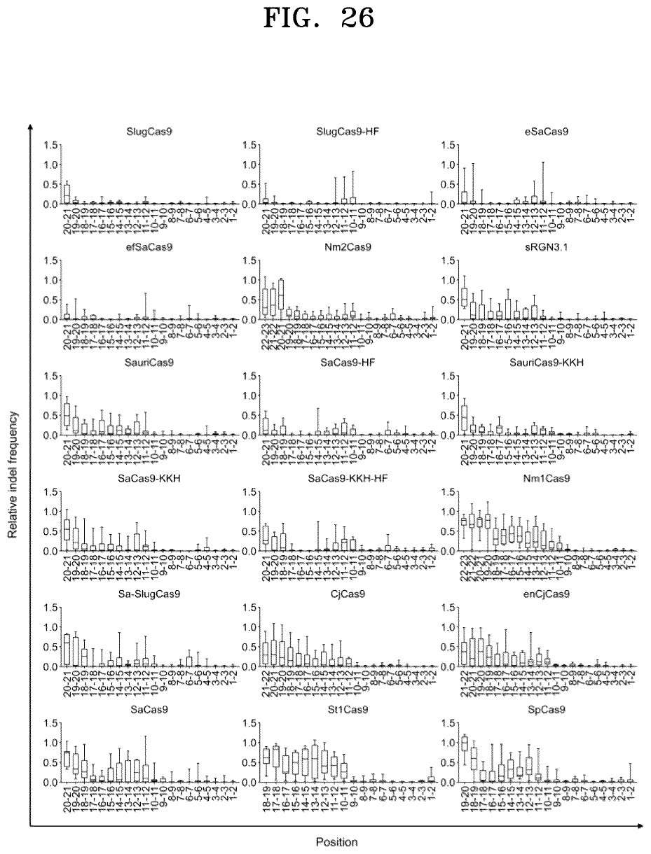

When we defined the specificity as 1—relative indel frequency (indel frequency at mismatched target sequence divided by that at perfectly matched target) as we did previously, we found that the general specificities of the Cas9s were as follows: 0.74 (SlugCas9), 0.72 (SlugCas9-HF), 0.70 (eSaCas9), 0.69 (efSaCas9), 0.65 (Nm2Cas9), 0.63 (sRGN3.1), 0.62 (SauriCas9), 0.56 (SaCas9-HF), 0.54 (SauriCas9-KKH), 0.53 (SaCas9-KKH), 0.52 (SaCas9-KKH-HF and Nm1Cas9), 0.50 (Sa-SlugCas9, CjCas9, and enCjCas9), 0.41 (SaCas9), and 0.35 (St1Cas9 and SpCas9) (FIG. 3A). However, given that Nm2Cas9 showed lower indel frequencies than did the other Cas9s at the ten matched target sequences, we cannot rule out the possibility that the measured specificity of 0.64 for Nm2Cas9 could be an overestimation. In general, the intolerance of the Cas9s for mismatches was higher in the half of the protospacer that is closer to the PAM than in the other half (FIG. 3A and FIG. 24). Among various types of mismatches, wobble transitions were tolerated the best and transversions the least (FIG. 3A and FIG. 25), a finding that is compatible with previous studies of Cas12a (or Cpf1), SpCas9, and SpCas9 variants. The relative specificity of the Cas9s followed a similar pattern for each type of mismatch: it was the highest with SlugCas9 and the lowest with St1Cas9 and SpCas9. When we evaluated the activities of the Cas9s at target sequences with consecutive two-base transversion mismatches, the activities dramatically decreased and the relative tolerance to mismatches was similar; it was the lowest with SlugCas9, SlugCas9-HF, eSaCas9, and efSaCas9 and the highest with Nm1Cas9, St1 Cas9, and SpCas9 (FIG. 3B and FIG. 26).

A comparison of the general activities and specificities of these Casts revealed a high-activity group containing sRGN3.1, SlugCas9, SaCas9, SpCas9, SauriCas9, Sa-SlugCas9, SaCas9-KKH, eSaCas9, and efSaCas9 and a low-activity group containing SauriCas9-KKH, SlugCas9-HF, SaCas9-HF, SaCas9-KKH-HF, St1Cas9, Nm1Cas9, enCjCas9, CjCas9, and Nm2Cas9 (FIG. 3C and FIG. 27). The general activity for each Cas9 was determined at target sequences with the primary PAM for the Cas9 and the general specificity was evaluated at targets with the primary or secondary PAM for the Cas9. Within the high-activity group, we observed a general trade-off between activity and specificity with two exceptions; the relative activities of sRGN3.1 and SlugCas9 were higher than expected given their relatively high specificities. The general activities of sRGN3.1, SlugCas9, and SaCas9 were significantly higher than that of SpCas9, which is in line with previous findings that the general activities of SaCas9 were slightly higher than or similar to those of SpCas9 at target sequences with the primary PAM for each Cas9. Importantly, when we compared the general activities and specificities of SpCas9 and the small Cas9s, both the general activities and specificities of sRGN3.1 and SlugCas9 were higher than those of SpCas9. In addition, the primary PAM for both sRGN3.1 and SlugCas9 is NNGG, which is very similar to NGG, the PAM for SpCas9. Thus, sRGN3.1 and SlugCas9 will frequently be preferred to SpCas9 for efficient and specific genome editing.

SaCas9-HF, eSaCas9, and efSaCas9, which are high fidelity variants derived from SaCas9, showed higher specificity than SaCas9. However, these variants, especially SaCas9-HF, revealed substantially lower activities than SaCas9. In line with this finding, the general activities of SlugCas9-HF and SaCas9-KKH-HF, which are high-fidelity variants derived from SlugCas9 and SaCas9-KKH, were substantially lower than those of SlugCas9 and SaCas9-KKH, respectively. Interestingly, however, the general specificities of these two high-fidelity variants were similar to those of the corresponding wild-type small Cas9s, suggesting that the engineering of these two small Cas9s has not substantially improved their fidelities.

In addition to examining the effects of mismatches, we also determined the effects of a 1-nt insertion or deletion in the target relative to the guide (resulting in a DNA or RNA bulge, respectively, in the target-guide pair) on the activities of two highly active small Cas9s, i.e., sRGN3.1 and SlugCas9, given that the targets with these insertions and deletions can be potential off-targets for SpCas9 and SaCas9. Because a previous study showed that the presence of such a DNA or RNA bulge drastically decreased the activity of SaCas9, we chose 75 guide sequences that can induce fairly high on-target editing efficiencies, which were paired with 137 target sequences with 0- to 3-bp mismatches or a 1-nt insertion or deletion at various positions (a total of 75×137=10,275 pairs of target and guide sequences). We found that the sRGN3.1- and SlugCas9-induced relative indel frequencies at targets with 1-nt insertions or deletions were lower than those with 1-bp mismatches, only slightly, albeit significantly, higher than those with 2-bp mismatches and higher than those with 3-bp mismatches (FIGS. 11A, 11B). Relatively low specificities were observed at mismatched targets with wobble base pairing, which is in line with findings described above, whereas the type of inserted or deleted base barely affected the specificity, except that the insertion of a G caused slightly lower relative activities (i.e., higher specificities) than the insertion of an A (FIGS. 11C-11L). Interestingly, the tolerance for an insertion or deletion differed depending on the position of the resulting bulge; the lowest relative activities of both sRNA3.1 and SlugCas9 were observed when the deletions were at positions 2-10 and when the insertions were at positions 11-15.

### Experimental Example 6. Computational Models to Predict the Activities of the Small Cas9s at Matched and Mismatched Target Sequences

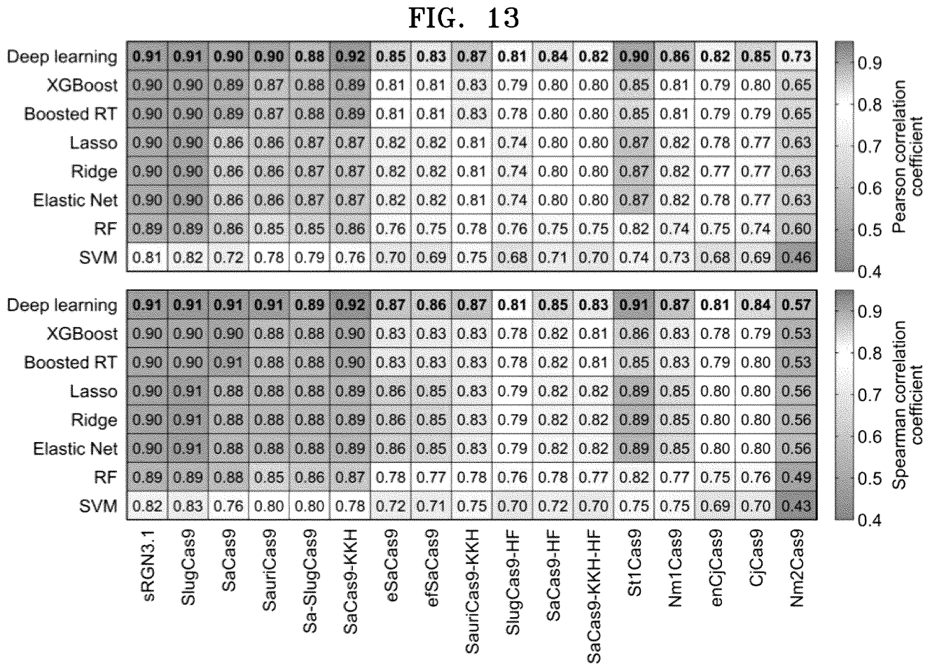

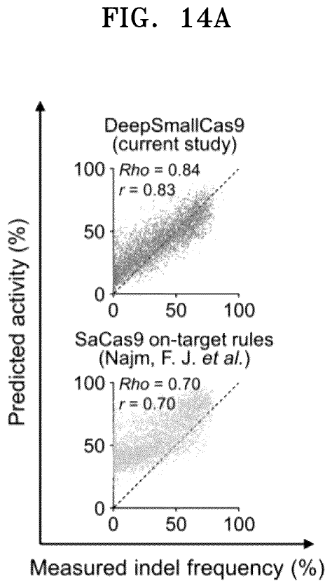

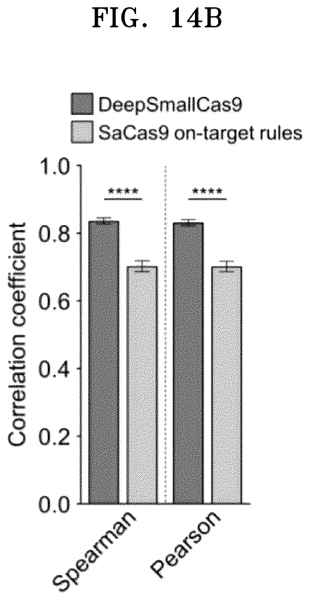

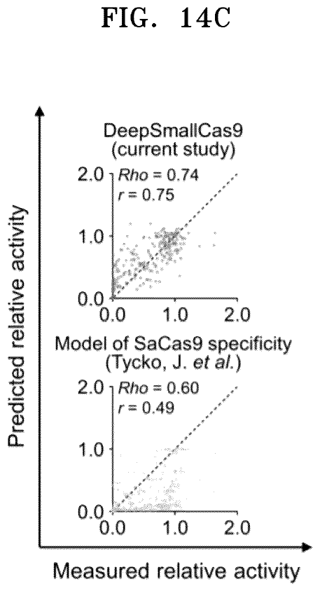

Choosing the most appropriate small Cas9 for editing a given genomic sequence is difficult because of the numerous possibilities; the selection process would be greatly facilitated by information about the predicted activity of each Cas9 at the given target sequence. Such predictions would be particularly valuable given that the relative activities of the small Cas9s can differ across target sequences as described above. We previously developed computational models for predicting the activities of AsCas12a, SpCas9, and SpCas9 variants at matched, but not mismatched, target sequences. To aid in the selection of appropriate small Cas9s for editing specific target sequences, we developed computational models that predict the activities of the 17 small Cas9s at matched and mismatched target sequences. Data about small Cas9-induced indel frequencies at matched targets and targets with mismatches, insertions, or deletions with all types of PAMs (primary, secondary, or inactive PAMs) from our study were randomly split into training and test datasets. As a result of this process, the training and test datasets shared almost no pairs of guide and target sequences; a small number of unintentionally shared pairs were manually removed from the test datasets. With the training datasets, we then developed seven conventional machine learning-based models and one deep learning-based computational model that predict the activities at both matched and mismatched target sequences for each small Cas9 (FIG. 12, FIG. 13). To improve the performance of these deep learning-based computational models, in addition to the guide and target sequences, we input information about the melting temperatures, MFEs, the numbers of G or C nucleotides, and the positions and types of mismatches between the guide and protospacer sequences. When the performances were compared using the Spearman and Pearson correlation coefficients, deep learning outperformed conventional machine learning for all 17 Cas9s. Thus, for subsequent model generation, we used deep learning for all of the small Cas9s.

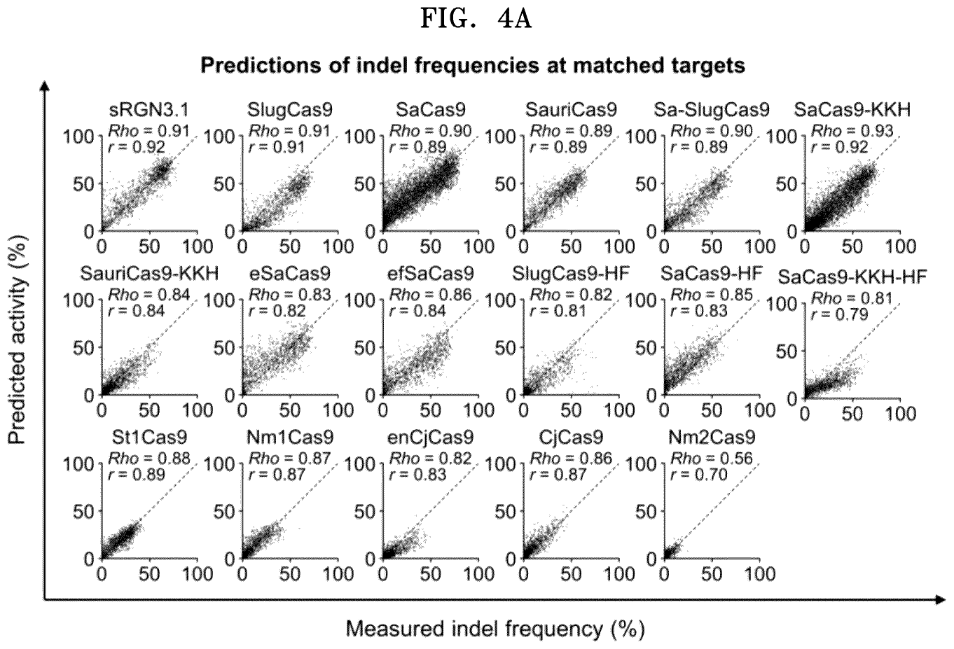

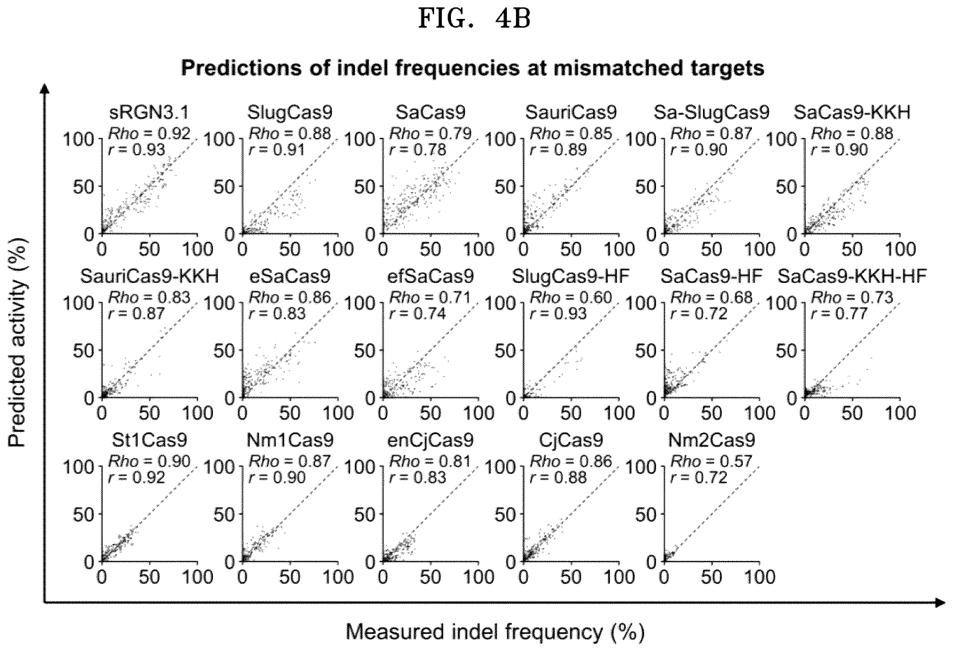

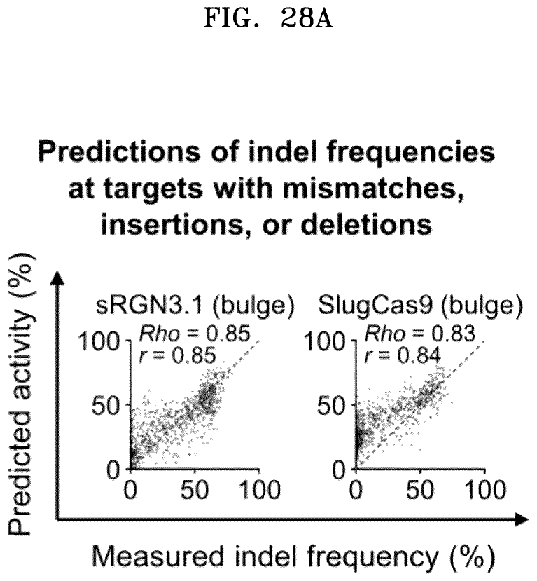

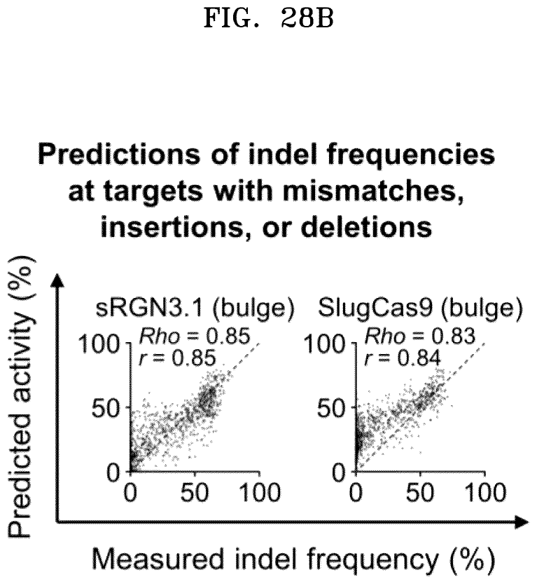

These deep learning-based computational models, collectively named DeepSmallCas9, were assessed using test datasets that had not been used for training. At matched target sequences, the Pearson correlation coefficients ranged from 0.70 to 0.92 (average 0.86, median 0.87) and the Spearman correlation coefficients ranged from 0.56 to 0.93 (average 0.86, median 0.87) (FIG. 4A and FIG. 28) and, at targets with mismatches, insertions, or deletions, the Pearson correlation coefficients ranged from 0.72 to 0.93 (average 0.85, median 0.87) and the Spearman correlation coefficients ranged from 0.57 to 0.92 (average 0.80, median 0.85) (FIG. 4B and FIG. 28), suggesting robust performances of these models.

In the case of SaCas9, two computational models, named “SaCas9 on-target rules” and “Model of SaCas9 specificity”, have been developed to predict efficiencies at matched and mismatched target sequences and were previously validated at target sequences containing NNGRR and NNGRRT PAMs, respectively. To compare the performance of DeepSmallCas9 with those of these two previously developed models, we generated subsets of our test datasets by filtering out matched target sequences that do not include an NNGRR PAM and mismatched targets that do not contain an NNGRRT PAM. When we compared the performances of the models using these subsets as test datasets, both the Spearman and the Pearson correlation coefficients of DeepSmallCas9 were higher than those of the previously developed models at matched and mismatched target sequences (FIG. 14), suggesting that DeepSmallCas9 is more accurate. Furthermore, DeepSmallCas9 is much more broadly applicable because it can be used with 16 other small Cas9s in addition to SaCas9 and because it does not have PAM-related restrictions.

In addition, to evaluate the activities of small Cas9s in cell lines other than HEK293T cells, we measured the activities of sRGN3.1, efSaCas9, SauriCas9, and Nm2Cas9 in DLD-1 and HCT116 cells in the high-throughput manner used with HEK293T cell lines. The relative activities of the four tested small Cas9s were the same across the tested cell lines (sRGN3.1>efSaCas9>SauriCas9-KKH>Nm2Cas9) and the measured activities were highly correlated with those predicted by DeepSmallCas9 in all cell lines although the absolute activities of the small Cas9s varied somewhat depending on the cell line (FIG. 15), These results suggest that DeepSmallCas9 is applicable to other cell lines that have not been used for model training. We provide DeepSmallCas9 as a web tool at <http://deepcrispr.info/DeepSmallCas9>, allowing readers to choose the most appropriate small Cas9 for their target sequences of interest.

Experimental Example 7. Computational Prediction of Preferred Small Cas9s at Diverse PAM Sequences

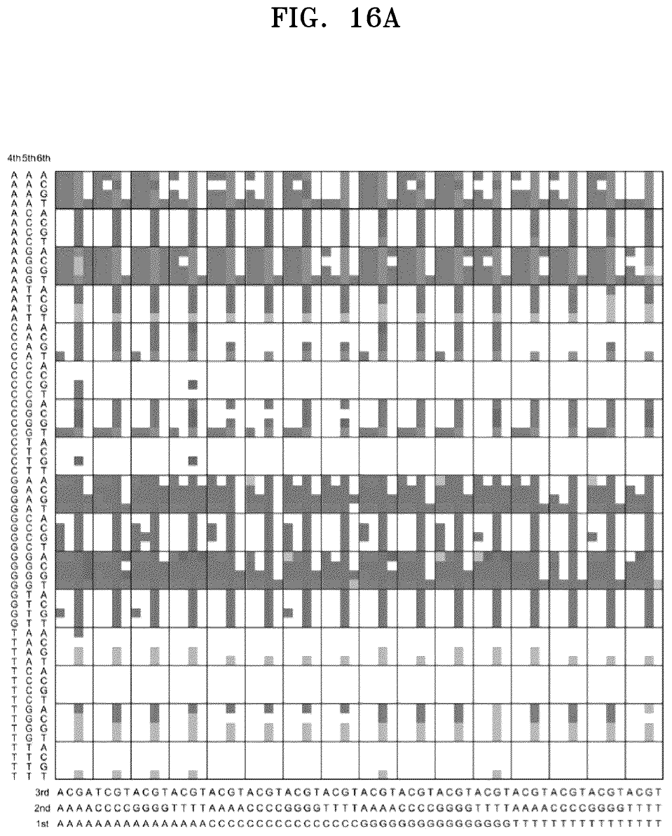

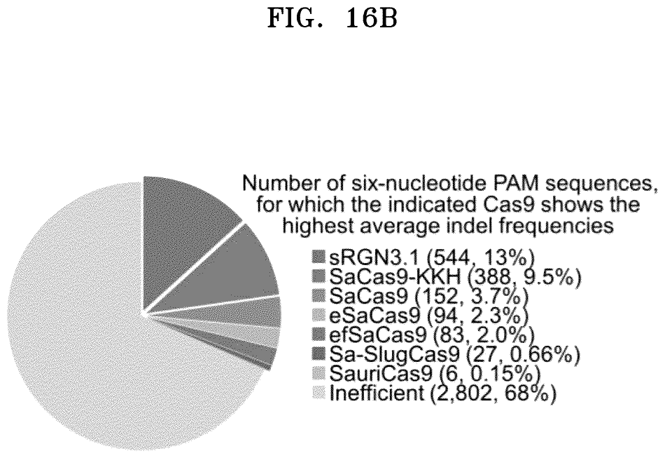

To examine PAM compatibilities over a broad range of sites, we used DeepSmallCas9 to predict the activities of the eight highly active small Cas9s, which include sRGN3.1, SlugCas9, SaCas9, SauriCas9, Sa-SlugCas9, SaCas9-KKH, eSaCas9, and efSaCas9, at a collection of 50 randomly designed protospacer sequences combined with all possible NNNNNN (4⁶=4,096) PAMs (i.e., a total of 204,800 target sequences). At least one of the small Cas9s was predicted to exhibit the average efficiencies higher than 10% at sites containing 1,294 out of the 4,096 PAMs (32%=1,294/4,096) (FIG. 16A, 16B), suggesting that target sequences with a wide range of PAM sequences can be efficiently targeted using one of the small Cas9s. When indel frequencies of 10% were chosen as the cutoff for useful genome editing, sRGN3.1 showed the widest PAM compatibilities (856/4,096=21%) (FIG. 16C).

### Experimental example 8. Choosing the Most Efficient and Specific Small Cas9 for Targeting Dominant Mutations

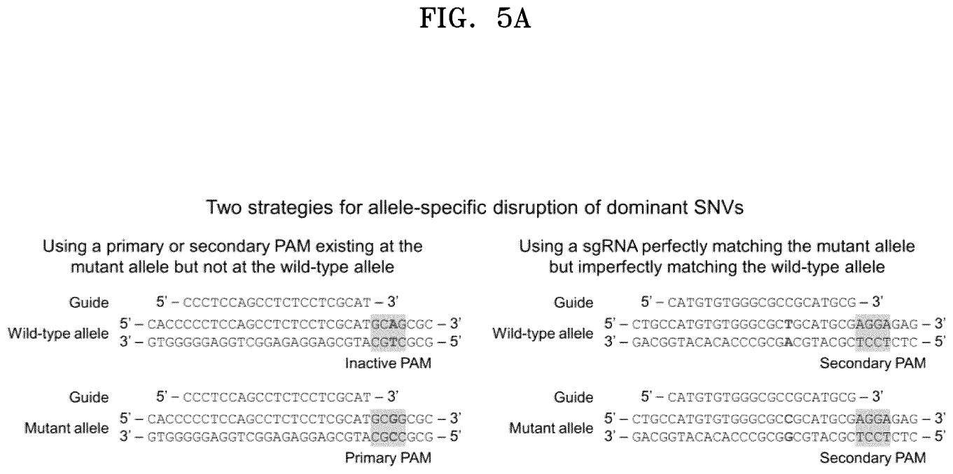

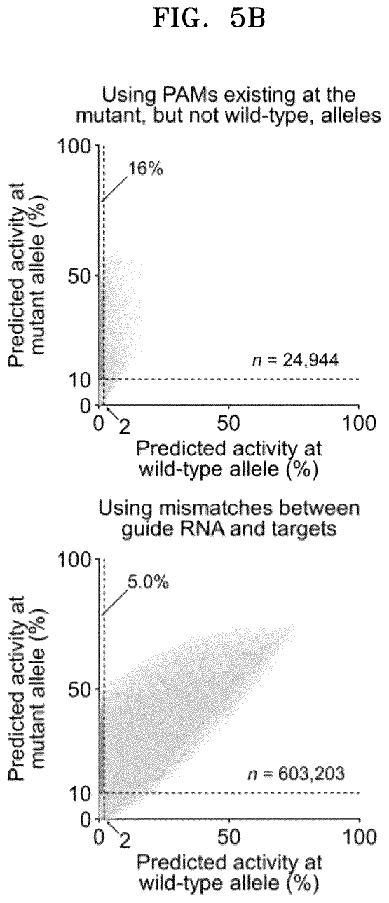

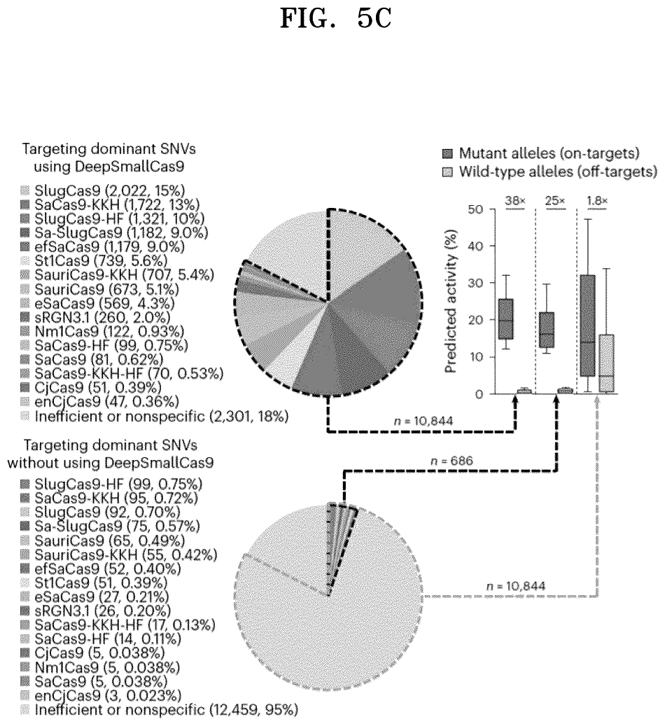

Diseases caused by dominant mutations can be ameliorated by selectively targeting such mutations. As an example of DeepSmallCas9 applications, we examined how many mutations out of the 13,145 dominant single nucleotide variants (SNVs) in protein-coding sequences reported in ClinVar could be targeted in an efficient and allele-specific manner with at least one of the small Cas9s. Allele-specificity based on a single-nucleotide difference can be achieved using a primary or secondary PAM existing at the mutant allele but not at the wild-type allele (strategy 1) or using a sgRNA perfectly matching the mutant allele but imperfectly matching the wild-type allele (strategy 2) (FIG. 5A). We calculated the predicted activities of 17 small Cas9s at the mutant alleles (on-targets) and the corresponding wild-type alleles (off-targets) using DeepSmallCas9. To identify efficient and mutant allele-specific small Cas9-sgRNA combinations for these mutations, we filtered out the combinations with on-target activity (activity at the mutant allele) lower than 10% and/or off-target activity (activity at the wild-type allele) higher than 2%, resulting in only 16% and 5.0% of the combinations designed by strategies 1 and 2, respectively, remaining (FIG. 5B), suggesting that, when these strategies are applied without using DeepSmallCas9, the majority (84% and 95% of those designed by strategies 1 and 2, respectively) of selected Cas9-sgRNA combinations are either inefficient or nonspecific in targeting the mutant allele. Thus, to identify efficient and specific combinations without using DeepSmallCas9, a large amount of experimentation testing the activities of many combinations at mutant and wild-type alleles would be required.

We found that 10,844 of the 13,145 mutations could be efficiently (on-target activity >10%) and allele-specifically (off-target activity <2%) targeted using at least one of the small Cas9s (FIG. 50), Then, to determine the most efficient and mutant allele-specific combination for each mutation, we added the on-target activity rank (in descending order) and the off-target activity rank (in ascending order), and selected the combination with the lowest value, as similarly conducted for SpCas9. Based on this analysis, SlugCas9. SaCas9-KKH, SlugCas9-HF, Sa-SlugCas9, and efSaCas9 were predicted to target 2,022 (15%), 1,722 (13%), 1,321 (10%), 1,182 (9.0%), and 1,179 (9.0%) SNVs, respectively, the combination of which represents 56% of all of the mutations, suggesting the usefulness of these five Cas9s for efficient and sequence-specific editing. sRGN3.1 was predicted to target only 260 (2.0%) SNVs potentially owing to its high activity even at the wild-type allele. These DeepSmallCas9-based selections of small Cas9 and sgRNA combinations were predicted to induce an average indel frequency of 21% (median, 20%; range, 10% to 67%) at the 10,844 mutant target sequences, which is 38-fold higher than the average indel frequency of 0.56% (median, 0.20%; range, 0.0% to 2.0%) predicted at the corresponding wild-type sequences. However, when we randomly chose small Cas9s and designed sgRNAs to distinguish the mutant and wild-type sequences via differences in the guide or PAM sequence for the 13,145 SNVs without using DeepSmallCas9, only 686 (5.2%) were targetable in an efficient and allele-specific manner. The expected average indel frequency for these 686 SNVs was 18% (median, 16%; range, 10% to 43%) at the mutant target sequences, which is 25-fold higher than the average indel frequency of 0.74% (median, 0.68%; range, 0.0% to 2.0%) predicted at the wild-type target sequences. When we randomly chose small Cas9s and designed sgRNAs for the 10,844 SNVs that can be efficiently and allele-specifically targeted using the DeepSmallCas9-based approach as described above, the expected average indel frequency for these 10,844 SNVs was 20% (median, 14%; range, 0.0% to 83%) at the mutant target sequences, which is only 1.8-fold higher than the average indel frequency of 11% (median, 4.8%; range, 0.0% to 81%) predicted at the wild-type target sequences, indicating low allele-specificity. Taken together, these results indicate that DeepSmallCas9 will greatly facilitate efficient and allele-specific genome editing using the small Cas9s.

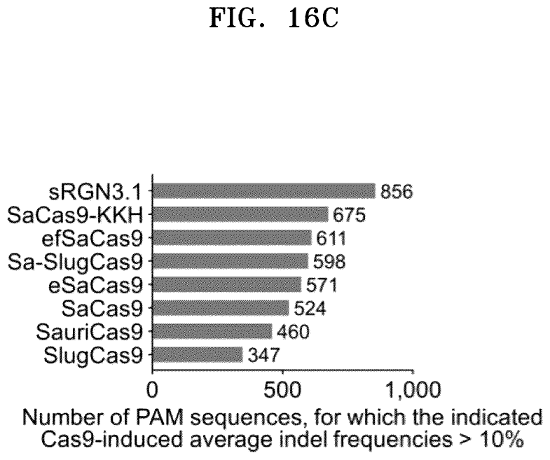

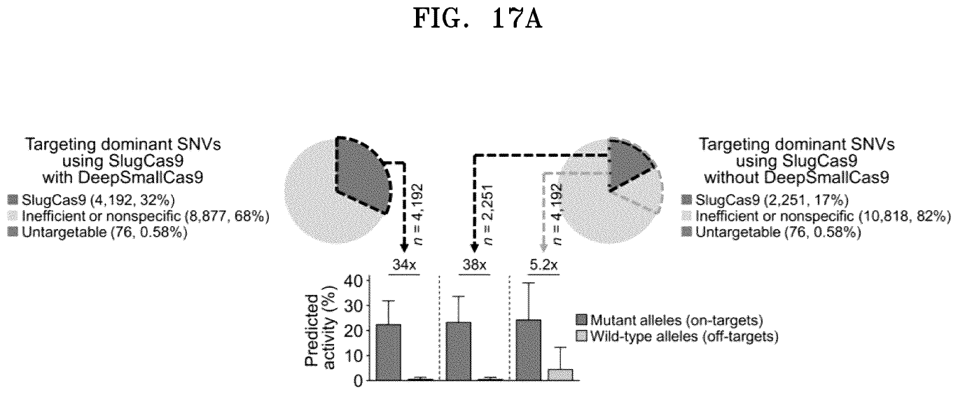

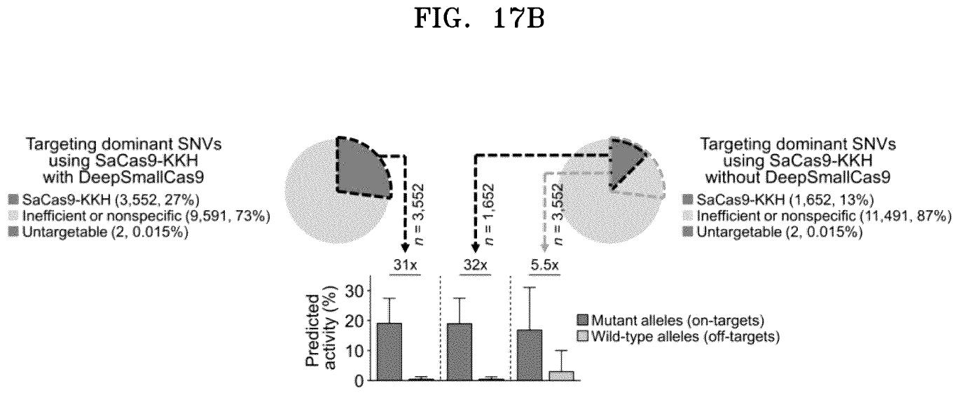

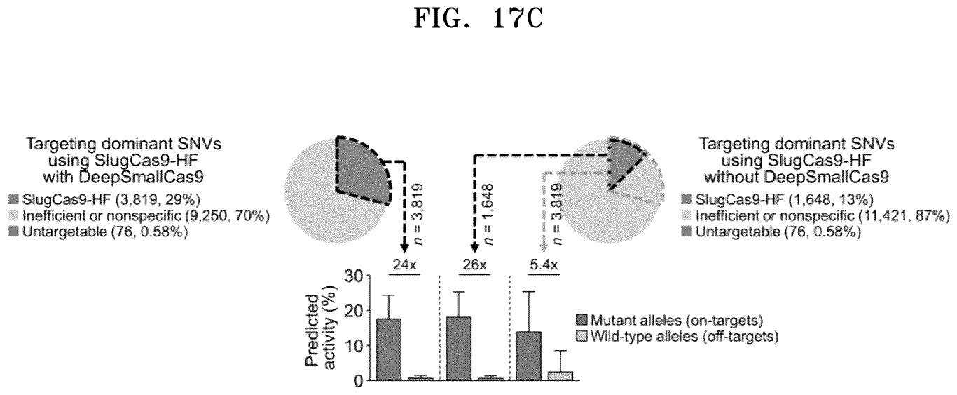

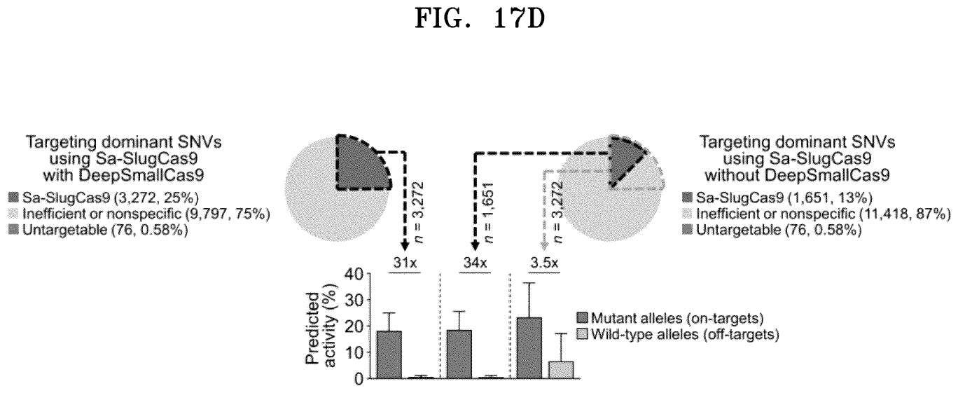

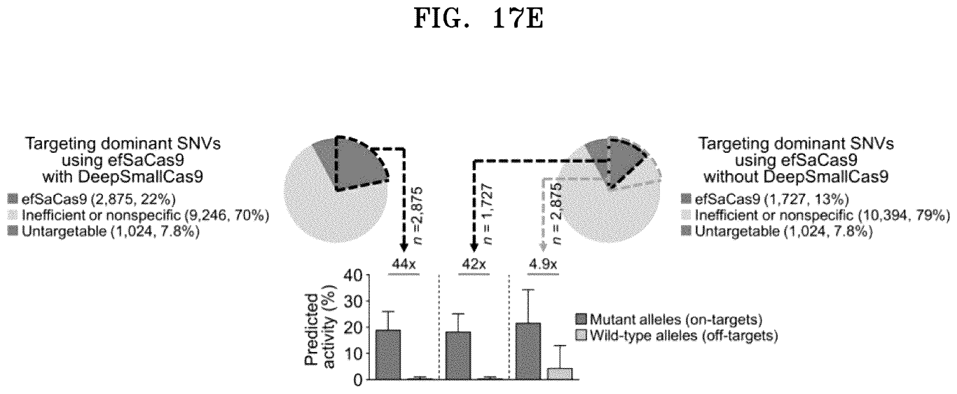

Furthermore, as another approach, we chose one small Cas9 out of the group of SlugCas9, SaCas9-KKH, SlugCas9-HF, Sa-SlugCas9, and efSaCas9, and designed mutant allele-specific sgRNAs such that the SNVs were located in regions in the target sequence with the following order of preference: i) the PAM, ii) the highly selective protospacer region (within 10 bp from the PAM), and iii) the remaining region in the protospacer. This rational design approach, not involving DeepSmallCas9, resulted in only 2,251 (17%), 1,652 (13%), 1,648 (13%), 1,651 (13%), and 1,727 (13%) out of the 13,145 mutations being targetable in an efficient and allele-specific manner when SlugCas9, SaCas9-KKH, SlugCas9-HF, Sa-SlugCas9, or efSaCas9 was chosen, respectively (FIG. 17). However, if we use DeepSmallCas9 to choose the sgRNAs, lugCas9, SaCas9-KKH, SlugCas9-HF, Sa-SlugCas9, and efSaCas9 could efficiently and selectively target 4,192 (32%), 3,552 (27%), 3,819 (29%), 3,272 (25%), and 2,875 (22%) SNVs, respectively, numbers that are 1.9-, 2.2-, 2.3-, 2.0-, and 1.7-fold higher than those obtained without using DeepSmallCas9. Taken together, these results suggest that DeepSmallCas9 will be useful for choosing an appropriate small Cas9-sgRNA combination for efficient and selective genome editing at given target sequences of interest.

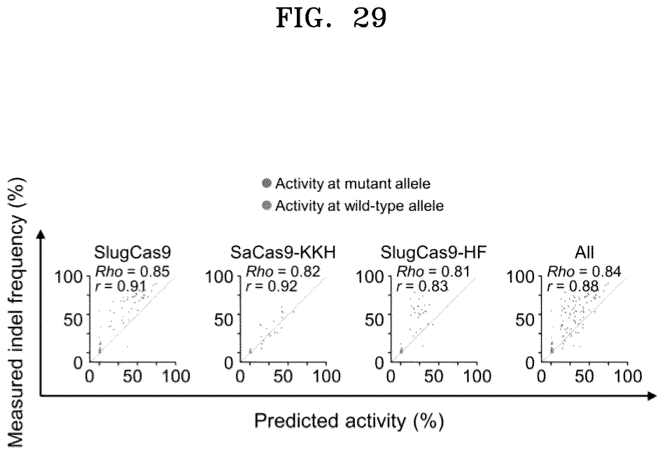

We next used DeepSmallCas9 to design sgRNAs for SlugCas9, SaCas9-KKH, and SlugCas9-HF (the three small Cas9s that were most frequently predicted to have high activities and specificities for targeting mutations reported in ClinVar as shown above) to target dominant pathogenic mutations. When we evaluated allele-specific targeting of these mutations, the analyzed 92 pairs of sgRNAs and small Cas9s showed high activities at the target sequences containing the dominant pathogenic mutations and low activities at the corresponding wild-type sequences, results that were highly correlated with the values predicted by DeepSmallCas9 (the Pearson correlation coefficients ranged from 0.83 to 0.92 (all combined, 0.88) and the Spearman correlation coefficients ranged from 0.81 to 0.85 (all combined, 0.84)) (FIG. 29), supporting that a DeepSmallCas9-mediated approach is useful for finding sgRNAs that target dominant pathogenic mutations.

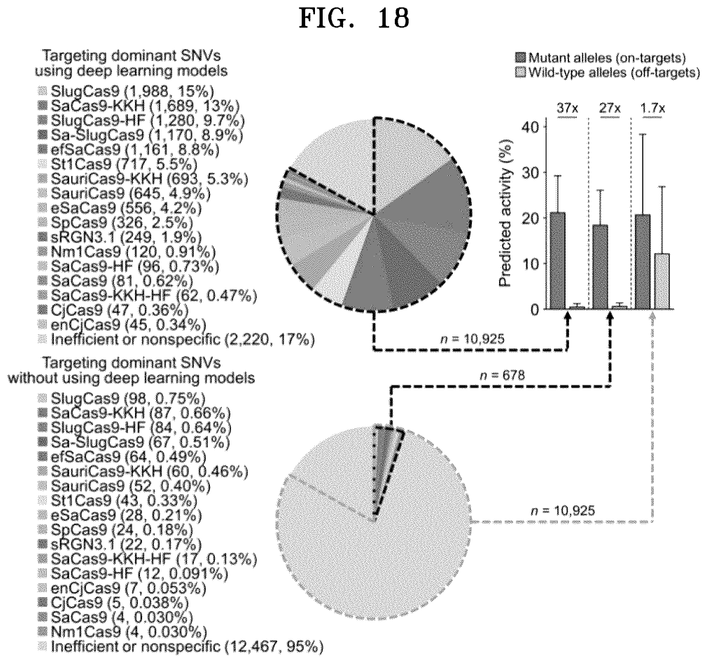

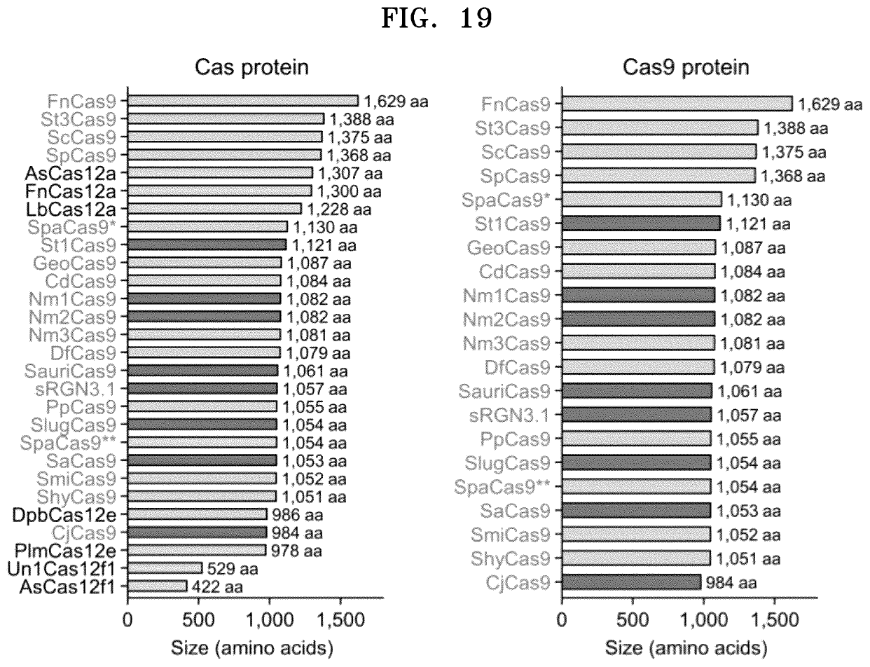

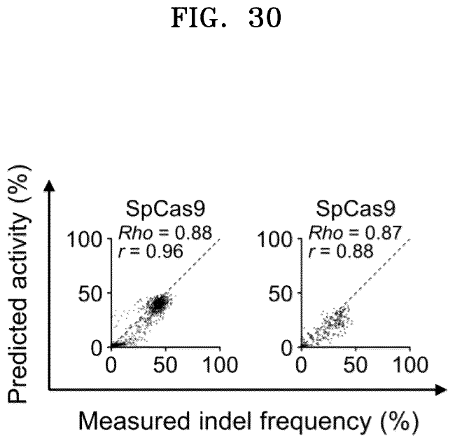

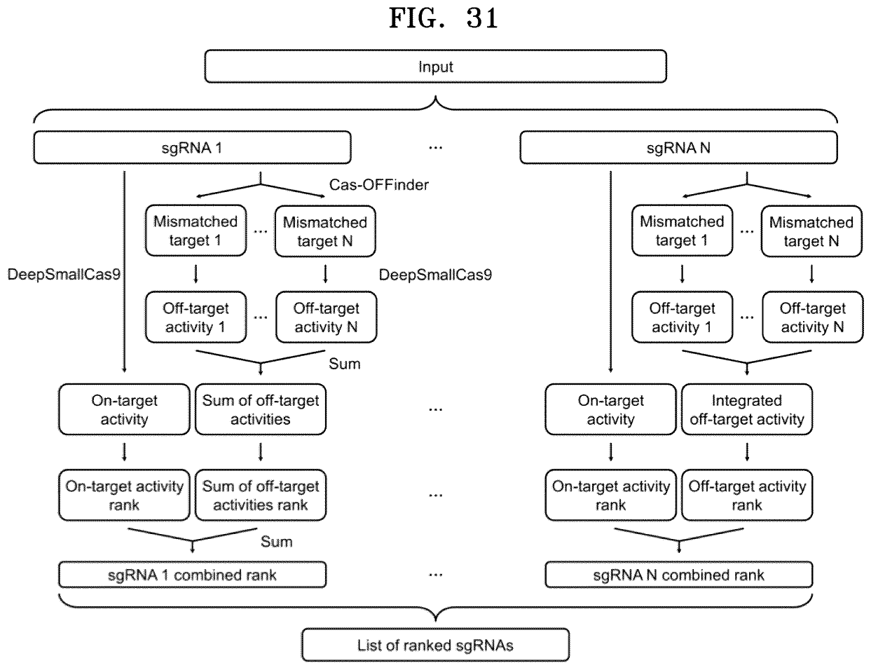

We also compared the activities and specificities of small Cas9s with those of SpCas9, which has been widely used for genome editing. As an example application, we attempted to target the 13,145 dominant mutations in an efficient and specific manner as described above. For this, we developed DeepSpCas9-v2, a deep-learning based computational model that predicts SpCas9 activities at matched and mismatched target sequences using the same algorithms used for DeepSmallCas9 and the SpCas9 activity data obtained in this study. DeepSpCas9-v2 showed robust performance (FIG. 30). We predicted the activities of the small Cas9s and SpCas9 at mutation-containing sequences (on-target) and the corresponding wild-type sequences (off-target) using DeepSmallCas9 and DeepSpCas9-v2 (FIG. 18). This addition of SpCas9 incrementally increased the number of mutations that could be efficiently and specifically targeted to 10,925 (+81 mutations, 0.62% increase). As the most efficient and specific combination of Cas9 and sgRNA per mutation, small Cas9s and SpCas9 were chosen for 10,599 (81%) and 326 (2.5%) SNVs, respectively, suggesting that small Cas9s will frequently be preferred over SpCas9 for efficient and sequence-specific genome editing as well as for AAV-mediated delivery.

The small Cas9 activity prediction system and method according to one aspect can predict the activities of 17 small Cas9s in matched or mismatched target sequences. It can be usefully used to provide information on a wide range of genome editing studies related to Cas9s, and small Cas9 and sgRNA that can specifically remove human single nucleotide mutations.

The above descriptions of the disclosure is provided only for illustrative purposes, and those of skill in the art will understand that the disclosure may be easily modified into other detailed configurations without modifying technical aspects and essential features of the disclosure. Hence, it should be understood that the above-described embodiments are not limiting of the scope of the disclosure.

1. A system for predicting an activity of small Cas9 using deep learning, comprising:
a sequence input unit receiving input data on a guide sequence and target sequence of small Cas9;
a predictive model generator generating a small Cas9 activity predictive model by performing deep learning for learning a relationship between small Cas9 activity data obtained from the input data on the guide sequence and target sequence of small Cas9 received from the sequence input unit and features that affect small Cas9 activity;
a candidate target sequence input unit receiving candidate target sequence of small Cas9; and
an activity predictor predicting small Cas9 activity by applying candidate target sequence input in the candidate target sequence input unit to the predictive model generated in the predictive model generator.

2. The system for predicting the activity of small Cas9 using deep learning according to claim 1, wherein the small Cas9 is any one selected from the group consisting of sRGN3.1, SlugCas9, SaCas9, SauriCas9, Sa-SlugCas9, SaCas9-KKH, eSaCas9, efSaCas9, SauriCas9-KKH, SlugCas9-HF, SaCas9-HF, SaCas9- KKH-HF, St1Cas9, Nm1Cas9, enCjCas9, CjCas9, and Nm2Cas9.

3. The system for predicting the activity of small Cas9 using deep learning according to claim 1, wherein the features that affect the small Cas9 activity include information on a melting temperature (Tm) calculated in different regions of the target sequence, a number of G or C nucleotides in a spacer and protospacer, a minimum free energy (MFE) of the spacer and sgRN, and a location and type of mismatch between the guide sequence and the protospacer sequence.

4. The system for predicting the activity of small Cas9 using deep learning according to claim 3, wherein the features that affect the small Cas9 activity further include information on an indel frequency of the target sequence.

5. The system for predicting the activity of small Cas9 using deep learning according to claim 4, wherein the indel frequency is calculated through Equation 1 below:
$$Indel frequency ( \% ) = \frac{\begin{matrix}Indel read counts - \\ ( Total read \times Background indel frequency )\end{matrix}}{\begin{matrix}Total read counts - \\ ( Total read \times Background indel frequency )\end{matrix}} \times 100 \qquad [ Equation 1 ]$$

6. The system for predicting the activity of small Cas9 using deep learning according to claim 1, wherein the predictive model generator generates a model for predicting the activity of small Cas9 through performing deep learning based on a convolutional neural network (CNN).

7. The system for predicting the activity of small Cas9 using deep learning according to claim 6, wherein the performing deep learning based on the convolutional neural network may include connecting the small Cas9 activity data and the features that affect the small Cas9 activity.

8. The system for predicting the activity of small Cas9 using deep learning according to claim 1, wherein the small Cas9 activity data is obtained by a method including:
infecting a cell line expressing small Cas9 with a lentiviral library containing oligonucleotides, each comprising a guide sequence and its corresponding target sequence;
performing deep sequencing by using DNA obtained from the cells into which the small Cas9 and lentiviral library have been introduced; and
measuring an indel frequency data from the data obtained by deep sequencing.

9. The system for predicting the activity of small Cas9 using deep learning according to claim 1, wherein the system for predicting the activity of small Cas9 further includes an output unit for outputting small Cas9 activity score predicted by the activity predictor.

10. The system for predicting the activity of small Cas9 using deep learning according to claim 1, wherein the target sequence includes a protospacer adjacent motif (PAM) sequence and a protospacer sequence.

11. A method for predicting the activity of small Cas9, comprising:
designing a target sequence of small Cas9; and
applying the target sequence designed by the designing above to the system for predicting the activity of small Cas9 according to claim 1.

12. A computer-readable recording medium having recorded thereon a program for causing a computer to execute a method for predicting the activity of small Cas9 according to claim 11.

13. A method for providing information on human single nucleotide mutations, comprising:
obtaining human single nucleotide variant data;
selecting data corresponding to pathogenic single nucleotide mutations among the human single nucleotide mutations; and
applying the selected data to the system for predicting the activity of small Cas9 according to claim 1.

14. The method for providing information on human single nucleotide mutation according to claim 13, wherein the applying the small Cas9 activity prediction system is to use a primary or secondary PAM existing at a mutant allele but not at a wild-type allele; or is to use a sgRNA perfectly matching the mutant allele but imperfectly matching the wild-type allele.
